(19) United States
(12) Patent Application Publication
(10) Pub. No.: US 2020/0207744 A1
(43) Pub. Date: Jul. 2, 2020

(54) DIMER COMPOUNDS, AND USE IN BINDING TOXIC REPEATS OF RNA

(71) Applicants: EXPANSION THERAPEUTICS, INC., San Diego, CA (US); THE SCRIPPS RESEARCH INSTITUTE, La Jolla, CA (US)

(72) Inventors: Timothy Allen Blizzard, Princeton, NJ (US); Matthew Disney, Jupiter, FL (US)

(21) Appl. No.: 16/728,248

(22) Filed: Dec. 27, 2019

Related U.S. Application Data
(60) Provisional application No. 62/786,002, filed on Dec. 28, 2018.

(51) Int. Cl. C07D 403/14; C07D 403/10; A61P 21/00

(52) U.S. Cl. CPC C07D 403/14; A61P 21/00; C07D 403/10

(57) ABSTRACT

Provided herein are compounds and methods for modulating abnormal repeat expansions of gene sequences. More particularly, provided are dimeric inhibitors of RNA and the uses of such inhibitors in regulating nucleotide repeat expansions, e.g., to treat Myotonic Dystrophy Type 1 (DM1), Myotonic Dystrophy Type 2 (DM2), Fuchs dystrophy, Huntington Disease, Amyotrophic Lateral Sclerosis, or Frontotemporal Dementia.

### BACKGROUND

Nucleotide repeat expansion disorders are caused by an inherited expansion of unstable repetitive elements in a gene or intron. The precise genetic nature of these diseases has only been recognized relatively recently, and as such, the development of drugs targeting these toxic nucleotide repeats is becoming an important strategy in addressing the need for additional therapies for the treatment of these disorders.

### SUMMARY

Provided herein are compounds and methods for modulating abnormal repeat expansions of gene sequences. More particularly, provided are binders of RNA and the uses of such binders in regulating nucleotide repeat expansions, e.g., to treat Myotonic Dystrophy Type 1 (DM1), Myotonic Dystrophy Type 2 (DM2), Fuchs dystrophy, Huntington Disease, Amyotrophic Lateral Sclerosis, or Frontotemporal Dementia.

The disclosure provides compounds of Formula I:

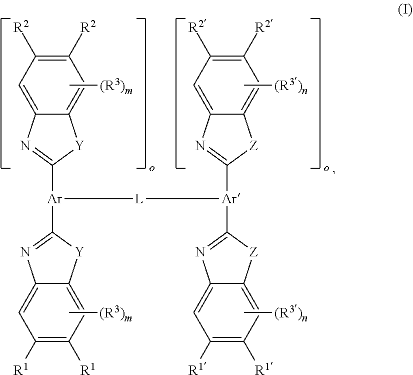

wherein

Ar and Ar′ are independently C₆ aryl or 5-12 membered heteroaryl having 1-4 heteroatoms selected from N, O, and S, wherein Ar and Ar′ are each optionally substituted with 1 to 4 R³;

each R¹, R¹′, R², and R²′ is independently selected from the group consisting of H, C₀₋₃ alkylene-halo, C₀₋₃ alkylene-CN, C₀₋₃ alkylene-NO₂, C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆ alkylene-R⁴;

each R³ and R³′ is independently selected from the group consisting of C₁₋₆alkyl, C₀₋₃ alkylene-halo, C₀₋₃ alkylene-CN, C₀₋₃ alkylene-NH₂, C₀₋₆ alkylene-NH(C═NH)NHR⁸, C₀₋₃ alkylene-OH, and C₀₋₃ alkylene-O—C₁₋₆alkyl;

each R⁴ is independently selected from the group consisting of H, C₁₋₆ alkyl, C₀₋₆alkylene-N(R⁶)₂, C₀₋₆alkylene-N(R⁶)₃⁺, C₀₋₆alkylene-NR⁷—C₀₋₆alkylene-N(R⁶)₂, CH(OR⁶)₂, C₀₋₆alkylene-OR⁶, C₀₋₆alkylene-O—C₀₋₆alkylene-N(R⁶)₂, C₀₋₆alkylene-NR⁶(C═NR⁶)N(R⁶)₂, C₀₋₆alkylene-NR⁶CO₂R⁶, C₀₋₆alkylene-NR⁶COR⁶, CH(R⁷)—C₀₋₆alkylene-NR⁶(C═NR⁶)N(R⁶)₂, and C₀₋₆alkylene-NR⁶C₀₋₆alkylene-NHCO—C₀₋₆alkylene-CO₂R⁶, or two R⁴ together with the nitrogen atom to which they are attached form a 4-7 membered heterocycloalkyl ring optionally further including one additional ring heteroatom selected from O and N;

each R⁶ is independently selected from the group consisting of H, C₁₋₆ alkyl, C₂₋₆ alkenyl, and C₂₋₆ alkynyl, or two R⁶s taken together with the heteroatom(s) to which they are attached form a 4-10 membered heterocycloalkyl ring, optionally having 1 to 3 additional heteroatom ring atom(s) selected from O and N, and said heterocycloalkyl ring is optionally substituted with one to three R⁷;

R⁷ is H, C₁₋₆ alkyl, C₀₋₆ alkyl-OR⁸, or CO₂R⁸;

R⁸ is H or C₁₋₆ alkyl;

each of Y and Z is independently NH or N—C₁₋₃alkyl;

L is —C₁₋₂₀alkylene-, —OC₁₋₂₀alkyleneO—, -(Q-C₁₋₄alkylene)_{y}Q-, —O—C₁₋₄alkylene-Het-C₁₋₄alkyleneO—, or —OC₁₋₄alkyleneO-Het-OC₁₋₄alkyleneO—, wherein the alkylene is optionally substituted with 1-3 groups selected from halo, OR⁸, CN, NO₂, N₃, NR⁸₂, SH, SCH₃, CO₂H, CONH₂, NH(C═NH)NHR⁸, C₆₋₁₀ aryl, 5 to 10-membered heteroaryl having 1-4 ring heteroatoms selected from N, O, and S, C₆₋₁₀ cycloalkyl, and 5 to 12-membered heterocycloalkyl having 1-3 ring heteroatoms selected from N, O, and S; and Het is a C₆aryl or 4-10 membered heterocycloalkyl or heteroaryl ring, having 1 to 4 heteroatom ring atom(s) selected from O, S, and N, and Het is optionally substituted with one to three R⁷;

each Q is independently O, S, NR⁸, CO₂, CONR⁸, NR⁸CO, NR⁸CONR⁸, NR⁸(C═NR⁸)NR⁸, NR⁸SO₂, SO₂, or SON(R⁸)₂;

each m and n is independently 0-2;

each o is independently 0 or 1; and

y is 1-6.

In some cases, the compounds are of Formula Ia, Ib, or Ic:

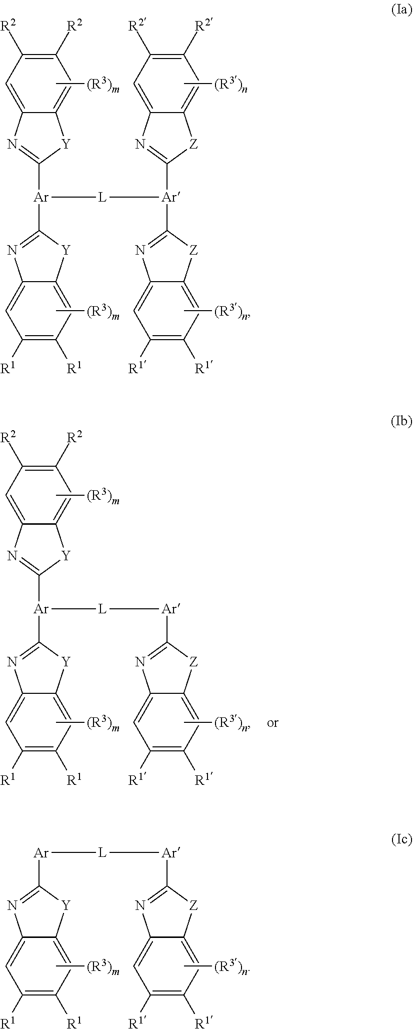

Also provided herein are compounds of Formula II:

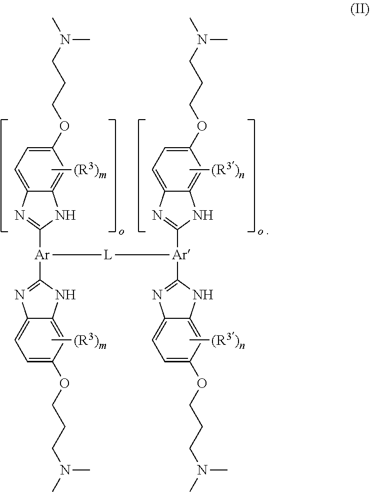

In some cases, the compounds are of Formula IIa, IIb, or IIc:

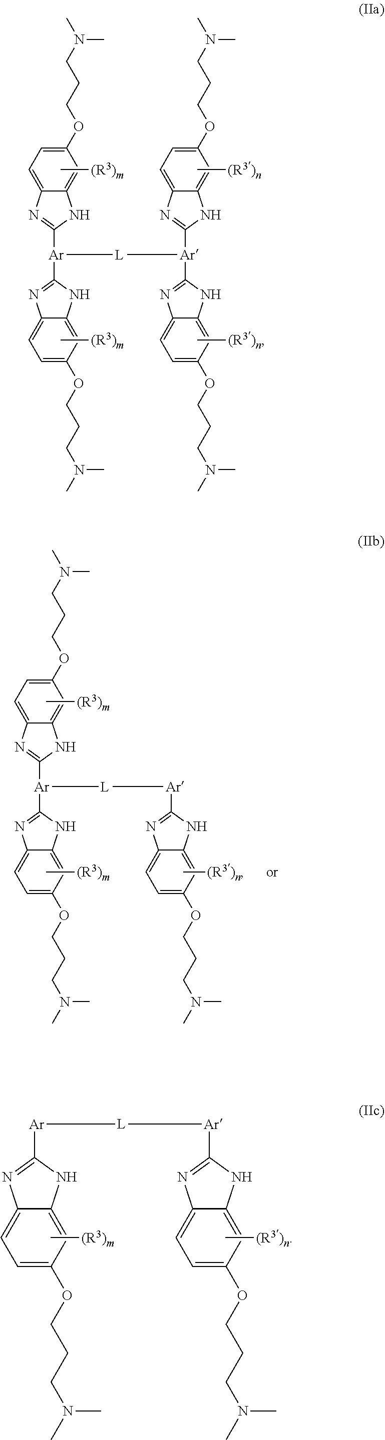

Further provided herein are methods of using the compounds disclosed to treat or prevent a nucleotide repeat disorder in a subject.

Other aspects of the disclosure include a compound as disclosed herein for use in the preparation of a medicament for treating or preventing a nucleotide repeat disorder in a subject, and the use of a compound as disclosed herein in a method of treating or preventing a nucleotide repeat disorder in a subject.

### DETAILED DESCRIPTION

Provided herein are nucleic acid-binding compounds that can treat or prevent a nucleotide repeat disorder in a subject. These compounds are useful in the treatment of a variety of diseases and disorders, including but not limited to, Myotonic Dystrophy Type 1, Myotonic Dystrophy Type 2, Fuchs dystrophy, Huntington Disease, Amyotrophic Lateral Sclerosis, and Frontotemporal Dementia.

### Compounds of the Disclosure

The disclosure provides compounds of Formula I:

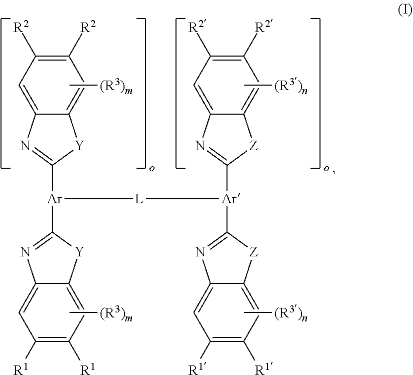

wherein Ar and Ar′ are independently C₆ aryl or 5-12 membered heteroaryl having 1-4 heteroatoms selected from N, O, and S, wherein Ar and Ar′ are each optionally substituted with 1 to 4 R³;

each R¹, R¹′, R², and R²′ is independently selected from the group consisting of H, C₀₋₃ alkylene-halo, C₀₋₃ alkylene-CN, C₀₋₃ alkylene-NO₂, C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆ alkylene-R⁴;

each R³ and R³′ is independently selected from the group consisting of C₁₋₆alkyl, C₀₋₃ alkylene-halo, C₀₋₃ alkylene-CN, C₀₋₃ alkylene-NH₂, C₀₋₆ alkylene-NH(C═NH)NHR⁸, C₀₋₃ alkylene-OH, and C₀₋₃ alkylene-O—C₁₋₆alkyl;

each R⁴ is independently selected from the group consisting of H, C₁₋₆ alkyl, C₀₋₆alkylene-N(R⁶)₂, C₀₋₆alkylene-N(R⁶)₃+, C₀₋₆alkylene-NR⁷—C₀₋₆alkylene-N(R⁶)₂, CH(OR⁶)₂, C₀₋₆alkylene-OR⁶, C₀₋₆alkylene-O—C₀₋₆alkylene-N(R⁶)₂, C₀₋₆alkylene-NR⁶(C═NR⁶)N(R⁶)₂, C₀₋₆alkylene-NR⁶CO₂R⁶, C₀₋₆alkylene-NR⁶COR⁶, CH(R⁷)—C₀₋₆alkylene-NR⁶(C═NR⁶)N(R⁶)₂, and C₀₋₆alkylene-NR⁶C₀₋₆alkylene-NHCO—C₀₋₆alkylene-CO₂R⁶, or two R⁴ together with the nitrogen atom to which they are attached form a 4-7 membered heterocycloalkyl ring optionally further including one additional ring heteroatom selected from O and N;

each R⁶ is independently selected from the group consisting of H, C₁₋₆ alkyl, C₂₋₆ alkenyl, and C₂₋₆ alkynyl, or two R⁶s taken together with the heteroatom(s) to which they are attached form a 4-10 membered heterocycloalkyl ring, optionally having 1 to 3 additional heteroatom ring atom(s) selected from O and N, and said heterocycloalkyl ring is optionally substituted with one to three R⁷;

R⁷ is H, C₁₋₆ alkyl, C₀₋₆ alkylene-OR⁸, or CO₂R⁸;

R⁸ is H or C₁₋₆ alkyl;

each of Y and Z is independently NH or N—C₁₋₃alkyl;

L is —C₁₋₂₀alkylene-, —OC₁₋₂₀alkyleneO—, -(Q-C₁₋₄alkylene)_{y}Q-, —O—C₁₋₄alkylene-Het-C₁₋₄alkyleneO—, or —OC₁₋₄alkyleneO-Het-OC₁₋₄alkyleneO—, wherein the alkylene is optionally substituted with 1-3 groups selected from halo, OR⁸, CN, NO₂, N₃, NR⁸₂, SH, SCH₃, CO₂H, CONH₂, NH(C═NH)NHR⁸, C₆₋₁₀ aryl, 5 to 10-membered heteroaryl having 1-4 ring heteroatoms selected from N, O, and S, C₆₋₁₀ cycloalkyl, and 5 to 12-membered heterocycloalkyl having 1-3 ring heteroatoms selected from N, O, and S; and Het is a C₆aryl or 4-10 membered heterocycloalkyl or heteroaryl ring, having 1 to 4 heteroatom ring atom(s) selected from O, S, and N, and Het is optionally substituted with one to three R⁷;

each Q is independently O, S, NR⁸, CO₂, CONR⁸, NR⁸CO, NR⁸CONR⁸, NR⁸(C═NR⁸)NR⁸, NR⁸SO₂, SO₂, or SON(R⁸)₂;

each m and n is independently 0-2;

each o is independently 0 or 1; and

y is 1-6.

In various embodiments, Ar and Ar′ are each independently 5-12 membered heteroaryl having 1-4 heteroatoms selected from N, O, and S and are optionally substituted with 1 to 4 R³. In various embodiments, Ar and Ar′ are each independently C₆ aryl and are optionally substituted with 1 to 4 R³. In some cases, Ar and Ar′ are each independently 5-10 membered heteroaryl having 1-4 heteroatoms selected from N, O, and S. In some cases, Ar and Ar′ are each independently 6 membered heteroaryl having 1-4 heteroatoms selected from N, O, and S. In some cases, Ar and Ar′ are each independently 9 membered heteroaryl having 1-4 heteroatoms selected from N, O, and S.

In various embodiments, each o is 0. In some cases, each o is 1. In some cases, one o is 0 and the other o is 1.

In various embodiments, the compounds have the structure of Formula Ia, Ib, or Ic:

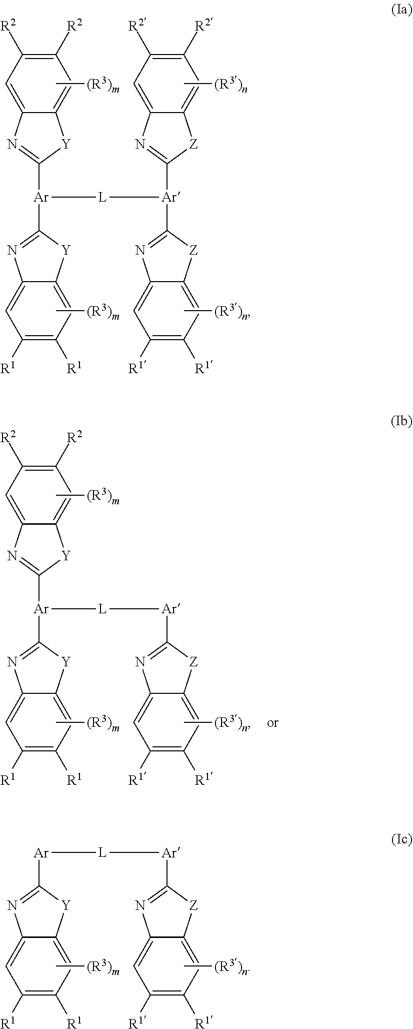

In various embodiments, the compounds have the structure of Formula II:

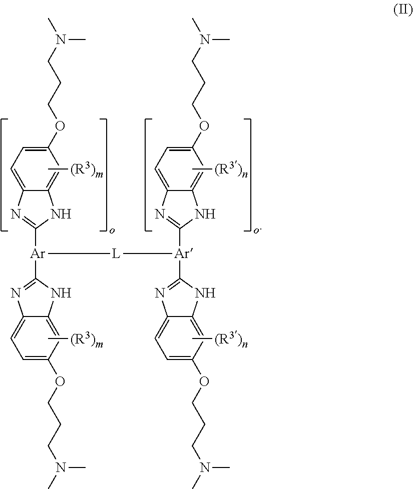

In various embodiments, the compounds have the structure of Formula IIa, IIb, or IIc:

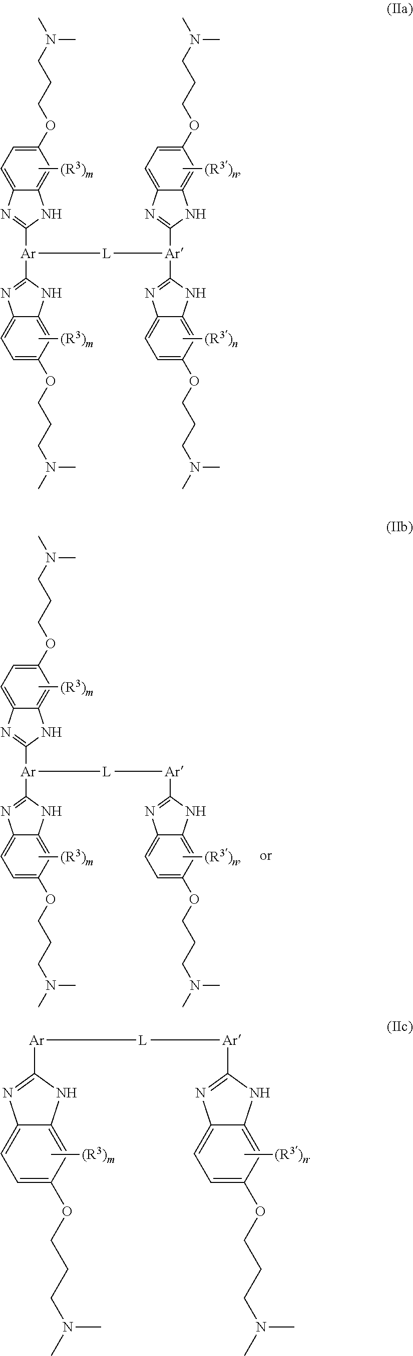

In various embodiments, L is

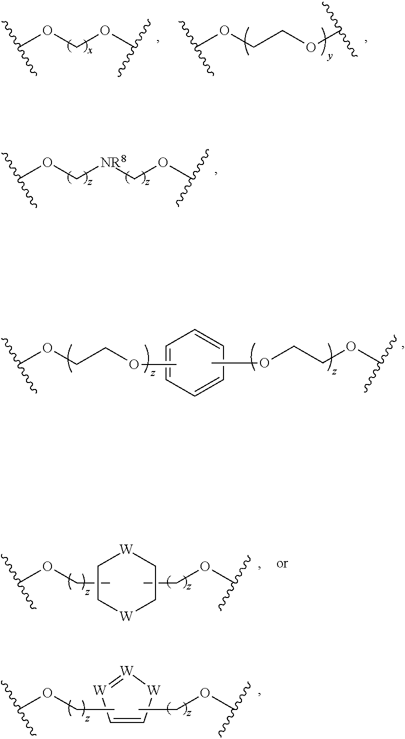

x is 1-14, z is 1-4, and each W is independently O, N, or NR⁸. In some cases, L is

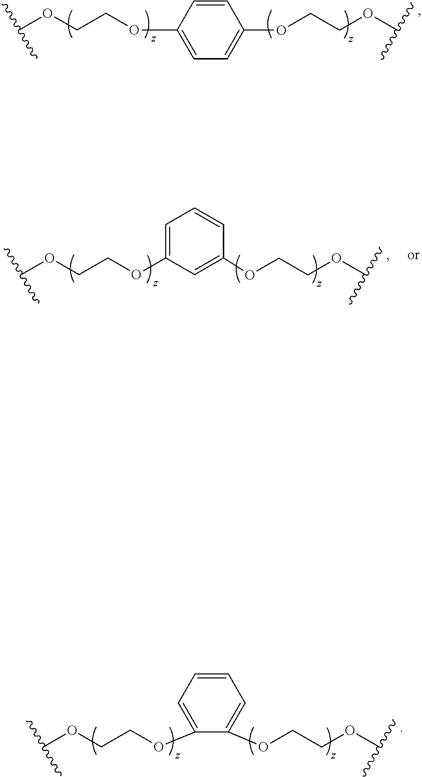

In some cases, L is

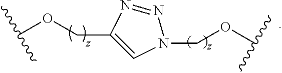

In some cases, L is

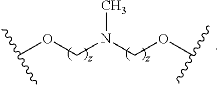

In some cases, L is —OC₁₋₁₂alkyleneO—.

In some cases, x is 1-14, 1-12, 1-10, 1-8, 1-6, or 1-4. In some cases, x is 2-14, 2-12, 2-10, 2-8, 2-6, or 2-4. In some cases, x is 1, 2, 3, 4, 5, 6, 7, 8, 9, 10, 11, 12, 13, or 14.

In some cases, y is 2-4. In some cases, y is 2, 3, or 4.

In some cases, z is 1-4, 1-3, or 1-2. In some cases, z is 2-4 or 2-3. In some cases, z is 1, 2, 3, or 4.

In some cases, at least one W is N. In some cases, at least one W is NR⁸. In some cases, one W is NR⁸. In some cases, two W are NR⁸. In some cases, at least one W is O. In some cases, one W is O. In some cases, two W are O.

In some cases, L is C₁₋₁₂alkylene. In some cases, L is C₁alkylene, C₂alkylene, C₃alkylene, C₄alkylene, C₅alkylene, C₆alkylene, C₇alkylene, C₈alkylene, C₉alkylene, C₁₀alkylene, C₁alkylene, or C₁₂alkylene.

In some cases, L is —OC₁₋₃alkylene —NR⁸—C₁₋₃alkyleneO—, —O—C₁₋₃alkylene-Het-C₁₋₃alkyleneO—, or —OC₁₋₃alkyleneO-Het-OC₁₋₃alkyleneO—. In some cases, L is —OC₂alkylene-NR⁸—C₂alkyleneO—, —O—C₂alkylene-Het-C₂alkyleneO—, or —OC₂alkyleneO-Het-OC₂alkyleneO—.

In some cases, each m and n are 1. In some cases, each m and n are 2. In some cases, at least one m or n is 0.

In some cases, each Y and Z is independently NH or NCH₃. In some cases, at least one Y or Z is NH. In some cases, both Y and Z are NH. In some cases, both Y and Z are NCH₃.

In some cases, one R¹ or R¹′ is selected from the group consisting of C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆ alkylene-R⁴, and the other R¹ or R¹′ is selected from the group consisting of H, C₀₋₃ alkylene-halo, C₀₋₃ alkylene-CN, C₀₋₃ alkylene-NO₂, C₀₋₆ alkylene-OR⁴, and C₀₋₆alkylene-SR⁴. In some cases, one R² or R²′ is selected from the group consisting of C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆ alkylene-R⁴, and the other R² or R²′ is selected from the group consisting of H, C₀₋₃ alkylene-halo, C₀₋₃alkylene-CN, C₀₋₃ alkylene-NO₂, C₀₋₆ alkylene-OR⁴, and C₀₋₆ alkyl-SR⁴.

In some cases, each R¹, R¹′, R², and R²′ is independently selected from the group consisting of H, C₀₋₃ alkylene-NH₂, C₀₋₆ alkylene-CON(R⁴)₂, C₀₋₆ alkylene-N(R⁴)₂, and C₀₋₆ alkylene-NH(C═NH)NHR⁴. In some cases, at least one R¹, R¹′ R², or R²′ comprises C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NHCOR⁴, C₀₋₆ alkylene-R⁴, and R⁴ comprises C₀₋₆alkylene-N(R⁶)₂, C₀₋₆alkylene-N(R⁶)₃⁺, C₀₋₆alkylene-NR⁷—C₀₋₆alkylene-N(R⁶)₂, or C₀₋₆alkylene-O—C₀₋₆alkylene-N(R⁶)₂. In some cases, one R¹ or R¹′ and one R² or R²′ each independently comprise C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NHCOR⁴, C₀₋₆ alkylene-R⁴, and R⁴ comprises C₀₋₆alkylene-N(R⁶)₂, C₀₋₆alkylene-N(R⁶)₃⁺, C₀₋₆alkylene-NR⁷—C₀₋₆alkylene-N(R⁶)₂, or C₀₋₆alkylene-O—C₀₋₆alkylene-N(R⁶)₂.

In some cases, R⁴ is selected from the group consisting of C₀₋₆alkylene-N(R⁶)₂, C₀₋₆alkylene-N(R⁶)₃⁺, C₀₋₆alkylene-NR⁷—C₀₋₆alkylene-N(R⁶)₂, and C₀₋₆alkylene-O—C₀₋₆alkylene-N(R⁶)₂.

In some cases, each R⁴ is independently selected from the group consisting of H, C₁₋₆ alkyl, C₂₋₆alkylene-N(R⁶)₂, C₂₋₆alkylene-N(R⁶)₃⁺, C₂₋₆alkylene-NR⁷—C₂₋₆alkylene-N(R⁶)₂, CH(OR⁶)₂, C₂₋₆alkylene-OR⁶, C₂₋₆alkylene-O—C₂₋₆alkylene-N(R⁶)₂, C₂₋₆alkylene-NR⁶(C═NH)N(R⁶)₂, C₂₋₆alkylene-NR⁶CO₂R⁶, C₂₋₆alkylene-NR⁶COR⁶, CH(R⁷)—C₂₋₆alkylene-NR⁶(C═NH)N(R⁶)₂, and C₂₋₆alkylene-NR⁶C₂₋₆alkylene-NHCO—C₂₋₆alkylene-CO₂R⁶.

In some cases, R⁶ comprises H or C₁₋₆alkyl, or two R⁶s taken together with the heteroatom(s) to which they are attached form a 4-10 membered heterocycloalkyl ring, optionally having 1 to 3 (e.g., 1-2, 1, 2, or 3) additional heteroatom ring atom(s) selected from O and N, and said heterocycloalkyl is optionally substituted with 1 or 2 R⁷.

Further provided are compounds as recited in Table A, or a pharmaceutically acceptable salt thereof.

TABLE A

| Com- | |
|---|---|
| pound | |
| # | Structure |
| A1 | 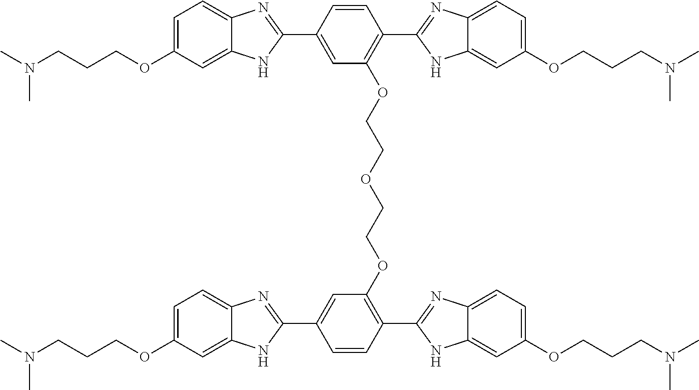 |
| A2 | 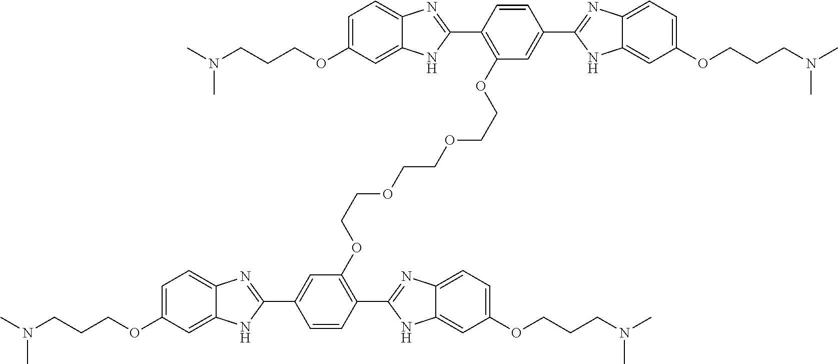 |
| A3 | 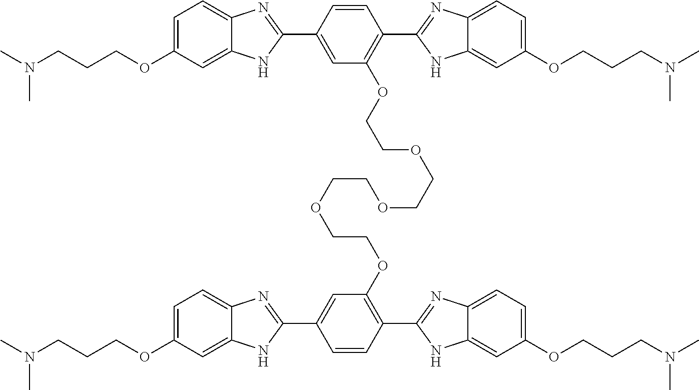 |
| A4 | 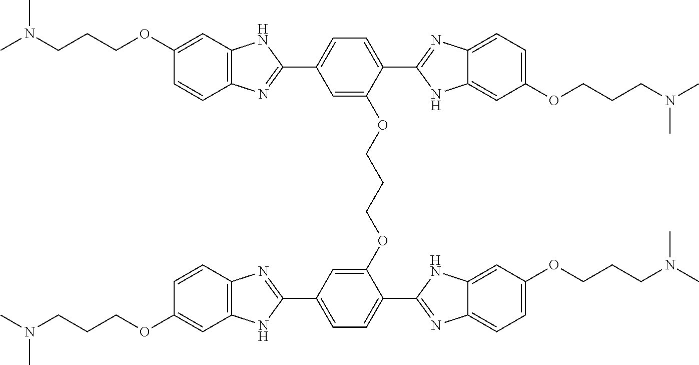 |
| A5 | 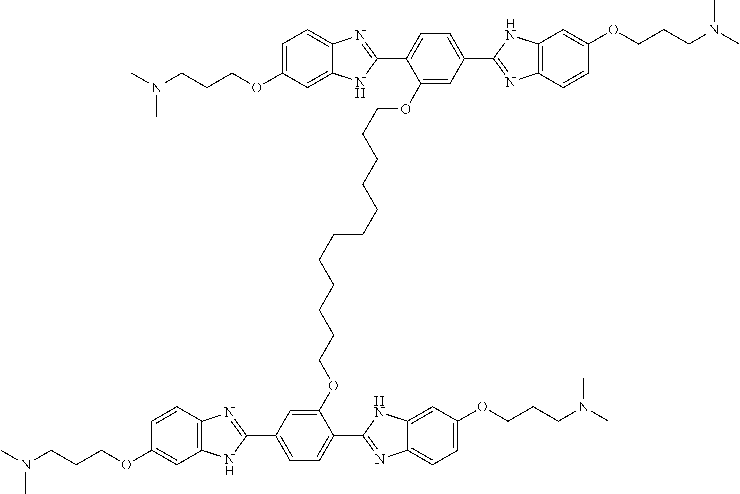 |
| A6 | 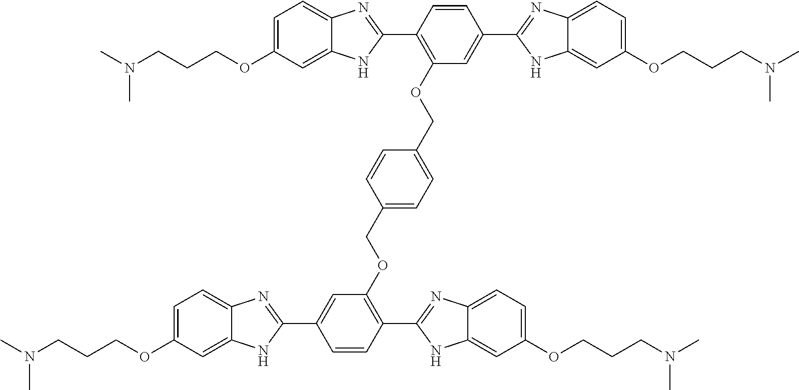 |
| A7 | 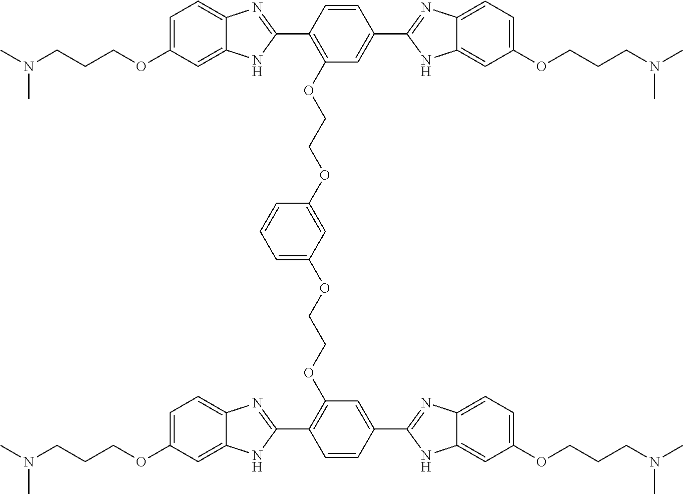 |
| A8 | 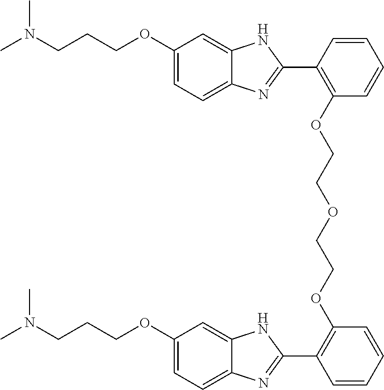 [SMILES: CN(C)CCCOc1ccc2nc(-c3ccccc3OCCOCCOc3ccccc3-c3nc4ccc(OCCCN(C)C)cc4[nH]3)[nH]c2c1] |
| A9 | 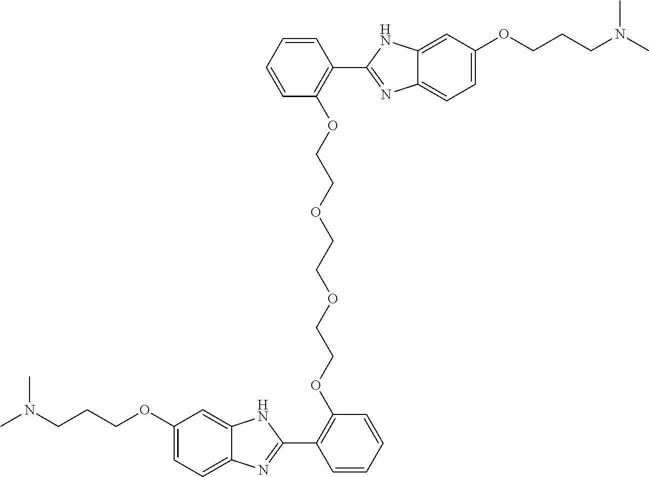 |
| A10 | 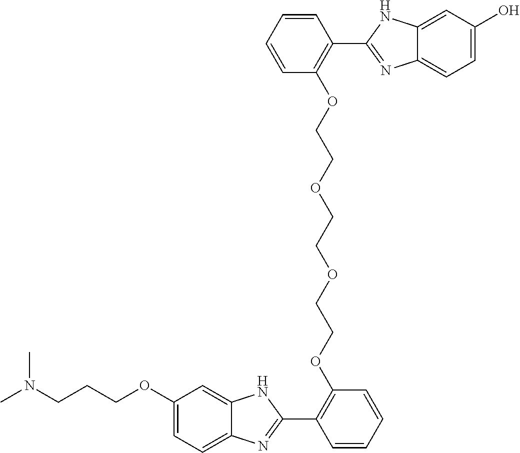 [SMILES: CN(C)CCCOc1ccc2nc(-c3ccccc3OCCOCCOCCOc3ccccc3-c3nc4ccc(O)cc4[nH]3)[nH]c2c1] |
| A11 | 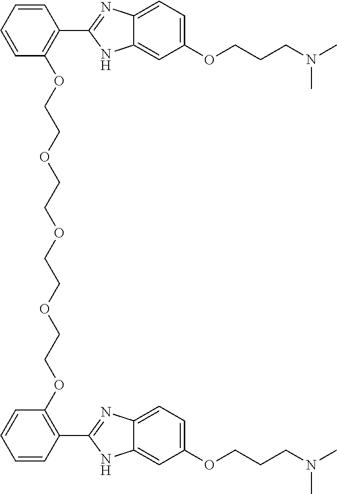 [SMILES: CN(C)CCCOc1ccc2nc(-c3ccccc3OCCOCCOCCOCCOc3ccccc3-c3nc4ccc(OCCCN(C)C)cc4[nH]3)[nH]c2c1] |
| A12 | 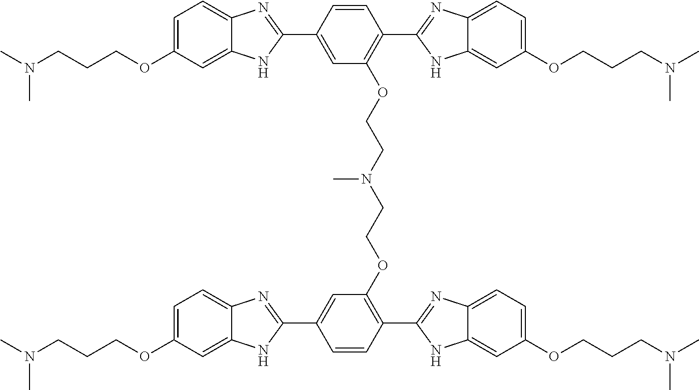 |
| A13 | 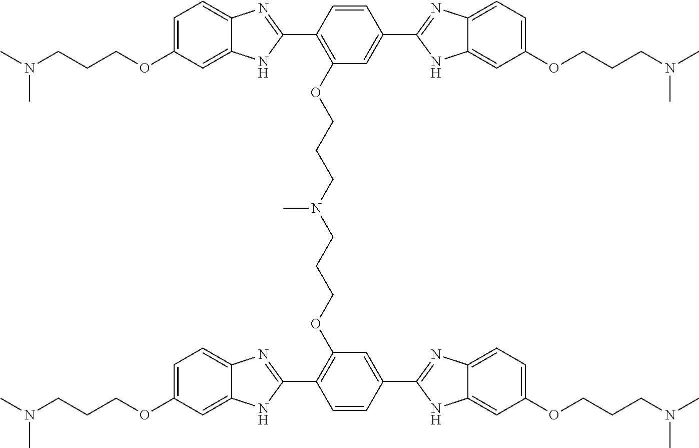 |
| A14 | 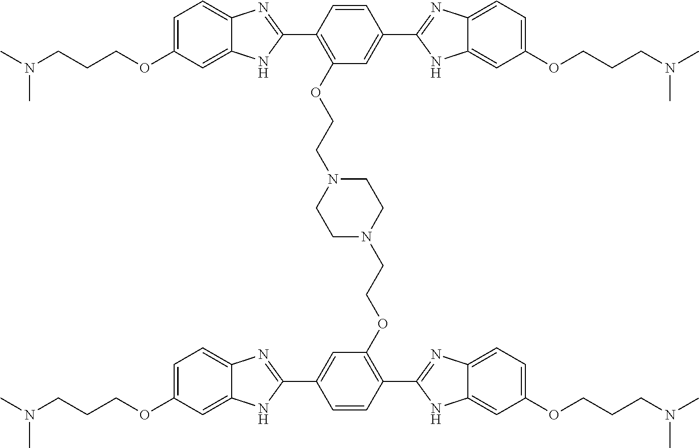 |
| A15 | 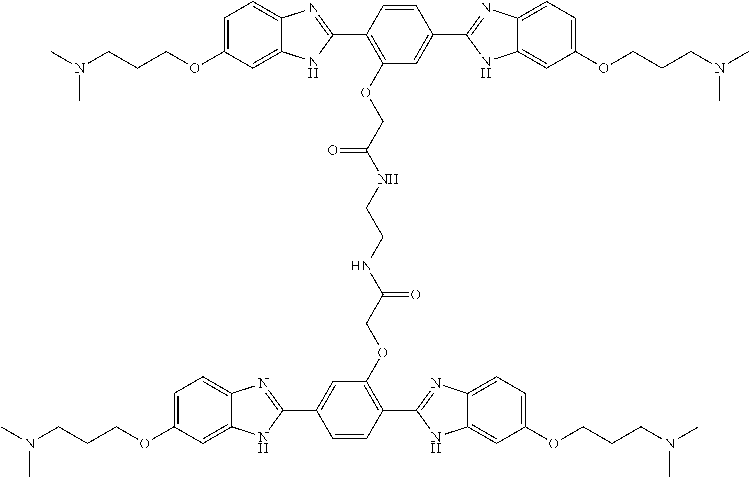 |
| A16 | 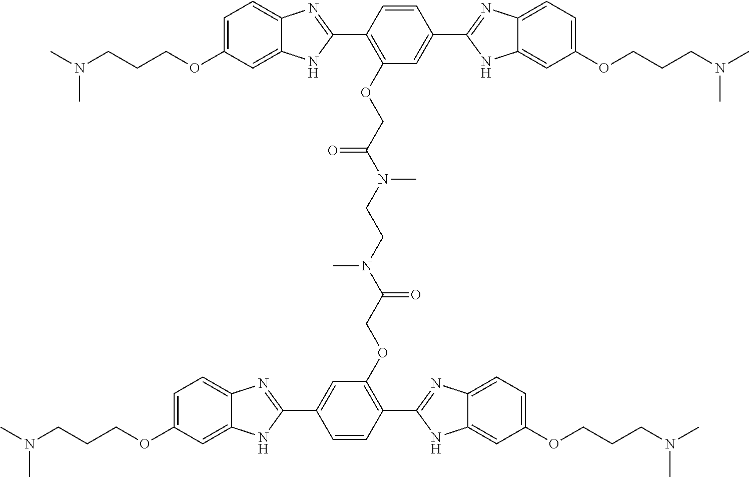 |
| A17 | 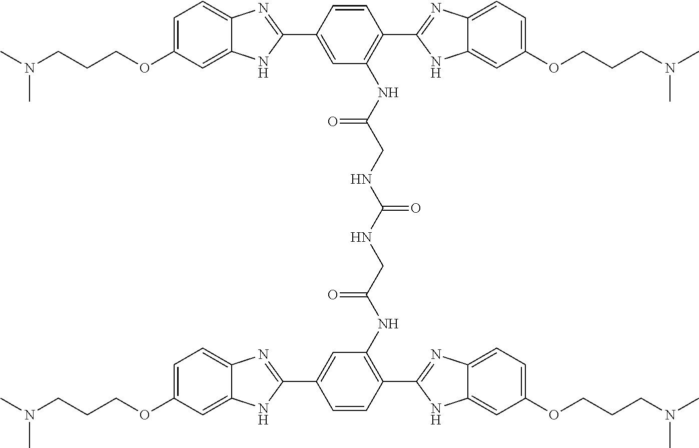 |
| A18 | 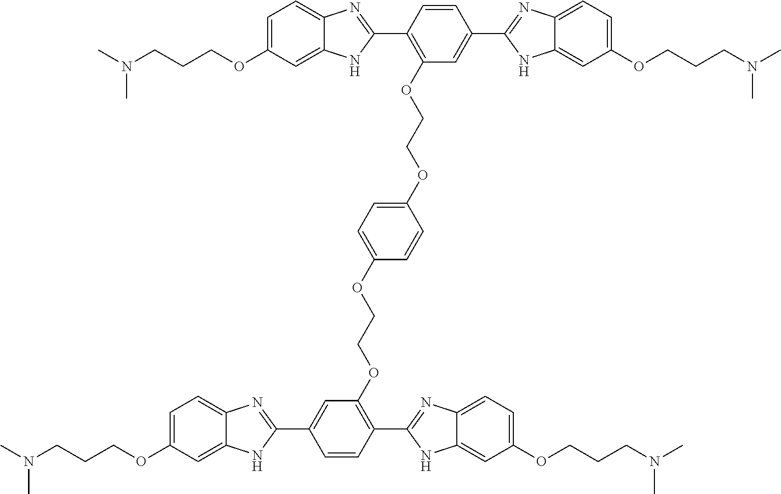 |
| A19 | 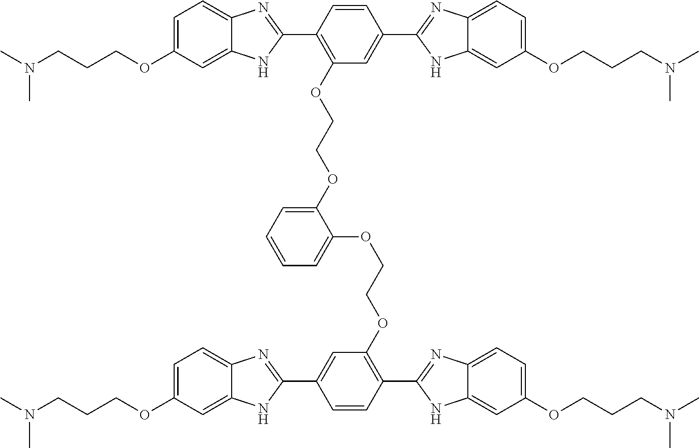 |
| A20 | 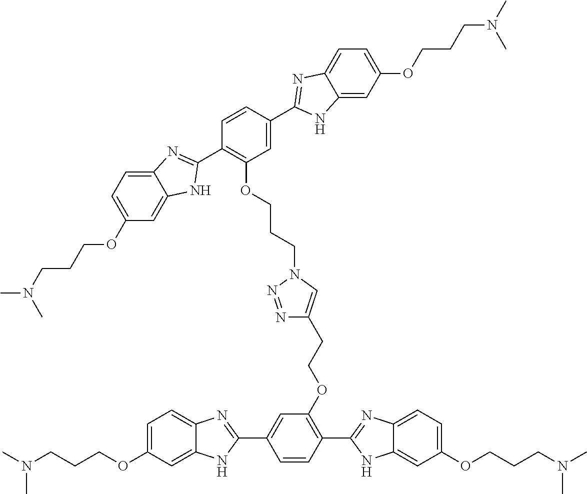 |
| A21 | 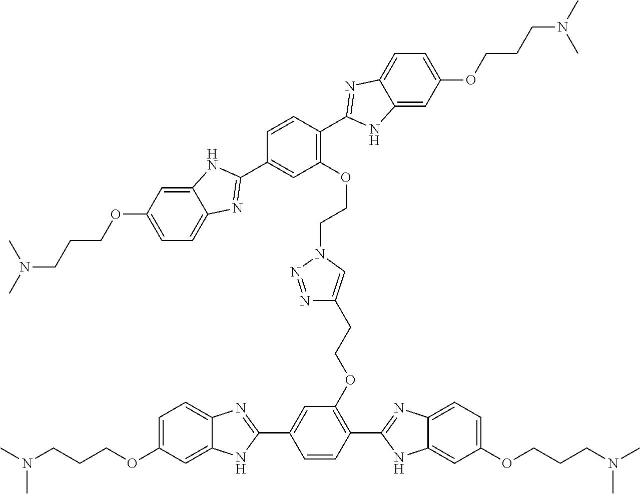 |
| A22 | 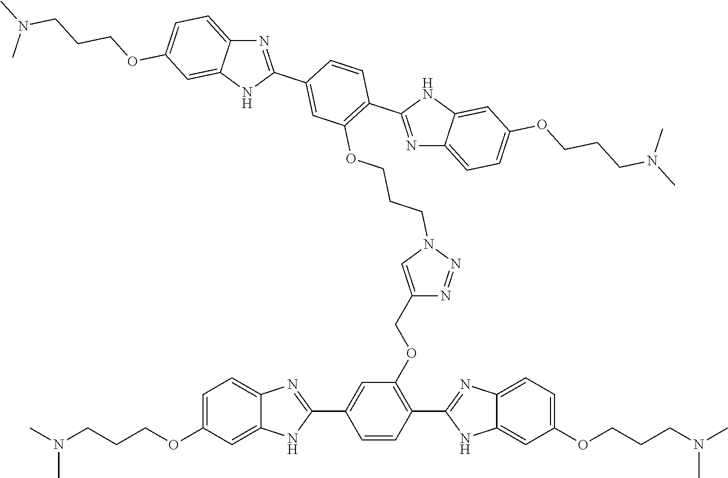 |
| A23 | 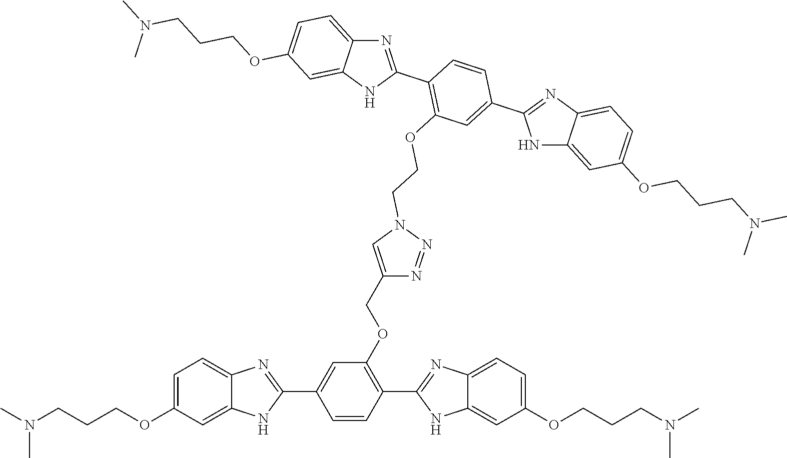 |
| A24 | 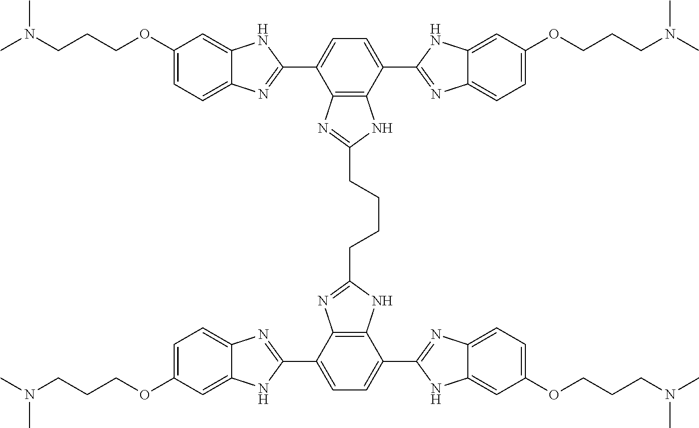 |
| A25 | 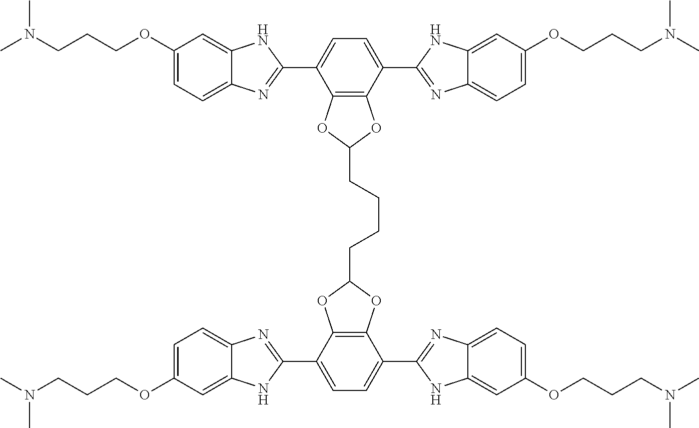 |
| A26 | 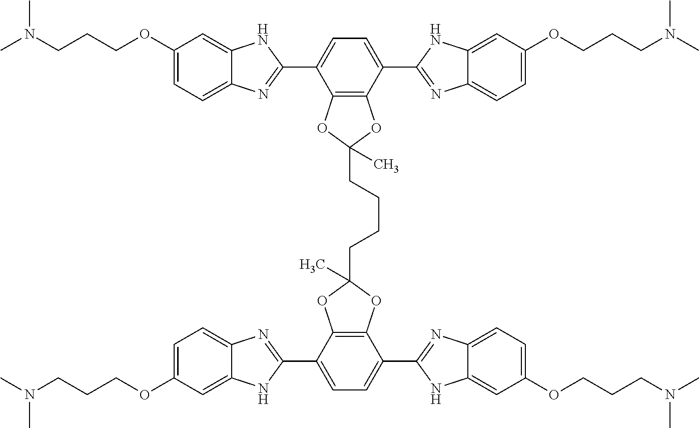 |
| A27 | 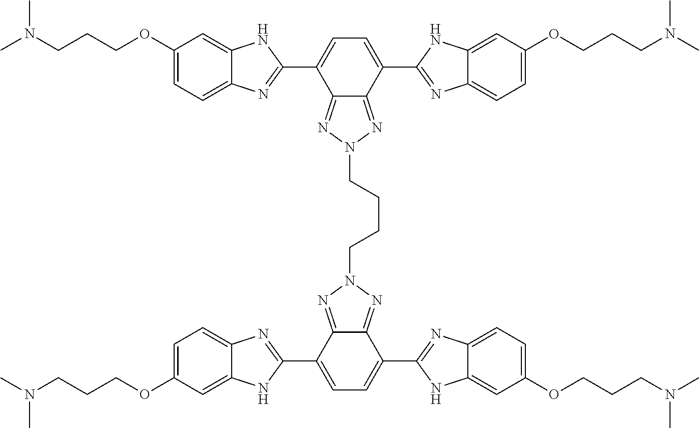 |
| A28 | 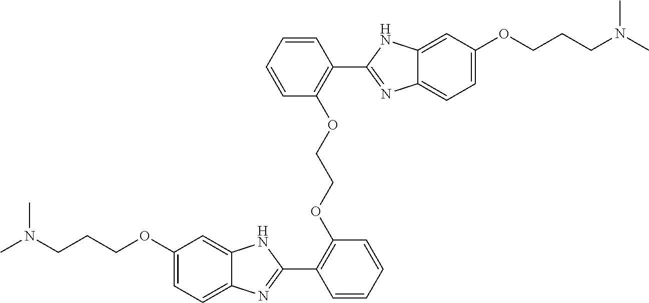 |

### Definitions

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₇ alkyl refers to an alkyl group having a number of carbon atoms encompassing the entire range (e.g., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkylene” used herein refers to an alkyl group having a substituent. For example, the term “alkylenehalo” refers to an alkyl group substituted with a halo group. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

The term “alkenyl” used herein refers to an unsaturated aliphatic group analogous in length and possible substitution to an alkyl group described above, but that contains at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. For example, a straight chain or branched alkenyl group can have six or fewer carbon atoms in its backbone (e.g., C₂-C₆ for straight chain, C₃-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). Unless otherwise indicated, an alkenyl group can be an unsubstituted alkenyl group or a substituted alkenyl group.

The term “alkynyl” used herein refers to an unsaturated aliphatic group analogous in length and possible substitution to an alkyl group described above, but that contains at least one triple bond. For example, the term “alkynyl” includes straight chain alkynyl groups (e.g., ethynyl, propynyl, butynyl, pentynyl, hexynyl, heptynyl, octynyl, nonynyl, decynyl), and branched alkynyl groups. For example, a straight chain or branched alkynyl group can have six or fewer carbon atoms in its backbone (e.g., C₂-C₆ for straight chain, C₄-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). Unless otherwise indicated, an alkynyl group can be an unsubstituted alkynyl group or a substituted alkynyl group.

As used herein, the term “cycloalkyl” refers to an aliphatic cyclic hydrocarbon group containing three to ten carbon atoms (e.g., 3, 4, 5, 6, 7, 8, 9, or 10 carbon atoms). The term Cₙmeans the cycloalkyl group has “n” carbon atoms. For example, C₅ cycloalkyl refers to a cycloalkyl group that has 5 carbon atoms in the ring. C₆-C₁₀ cycloalkyl refers to cycloalkyl groups having a number of carbon atoms encompassing the entire range (e.g., 6 to 10 carbon atoms), as well as all subgroups (e.g., 6-7, 6-8, 7-8, 6-9, 6, 7, 8, 9, and 10 carbon atoms).

Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group. When a cycloalkyl group is fused to another cycloalkyl group, then each of the cycloalkyl groups can contain three to ten carbon atoms unless specified otherwise. Unless otherwise indicated, a cycloalkyl group can be unsubstituted or substituted.

As used herein, the term “heterocycloalkyl” is defined similarly as cycloalkyl, except the ring contains one to four heteroatoms independently selected from oxygen, nitrogen, and sulfur. In particular, the term “heterocycloalkyl” refers to a ring containing a total of three to ten atoms (e.g., five to ten), of which 1, 2, 3 or 4 of those atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like. Cycloalkyl and heterocycloalkyl groups can be saturated or partially unsaturated ring systems optionally substituted with, for example, one to three groups, independently selected from alkyl, alkyleneOH, CO₂H, CO₂alkyl, C(O)NH₂, NH₂, oxo (═O), aryl, alkylenehalo, halo, and OH. Other substituents are also contemplated, including C₀₋₃ alkylene-halo, C₀₋₃ alkylene-CN, C₀₋₃ alkylene-NH₂, C₀₋₃ alkylene-OH, and C₀₋₃ alkylene-O—C₁₋₃alkyl. Heterocycloalkyl groups optionally can be further N-substituted with alkyl, alkylene-OH, alkylenearyl, and alkyleneheteroaryl. The heterocycloalkyl groups described herein can be isolated or fused to another heterocycloalkyl group, a cycloalkyl group, an aryl group, and/or a heteroaryl group. When a heterocycloalkyl group is fused to another heterocycloalkyl group, then each of the heterocycloalkyl groups can contain three to ten total ring atoms, and one to three heteroatoms. Unless otherwise indicated, a heterocycloalkyl group can be unsubstituted or substituted.

As used herein, the term “aryl” refers to a monocyclic aromatic group, such as phenyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Other substituents are also contemplated, including C₀₋₃ alkylene-halo, C₀₋₃ alkylene-CN, C₀₋₃ alkylene-NH₂, C₀₋₆ alkylene-NH(C═NH)NH₂, C₀₋₆ alkylene-NH(C═NH)NHalkyl, C₀₋₆ alkylene-NH(C═NH)N(alkyl)₂, C₀₋₃ alkylene-OH, and C₀₋₃ alkylene-O—C₁₋₆alkyl. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or polycyclic aromatic ring having 5 to 12 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, ON, NC, OH, alkoxy, amino, CO₂H, CO₂alkyl, aryl, and heteroaryl. Other substituents are also contemplated, including C₀₋₃ alkylene-halo, C₀₋₃ alkylene-CN, C₀₋₃ alkylene-NH₂, C₀₋₆ alkylene-NH(C═NH)NH₂, C₀₋₆ alkylene-NH(C═NH)NHalkyl, C₀₋₆ alkylene-NH(C═NH)N(alkyl)₂, C₀₋₃ alkylene-OH, and C₀₋₃ alkylene-O—C₁₋₆alkyl. In some cases, the heteroaryl group is substituted with one or more of alkyl and alkoxy groups. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “alkoxy” or “alkoxyl” as used herein refers to a “—O-alkyl” group. The alkoxy or alkoxyl group can be unsubstituted or substituted.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the term ““linker” refers to a bond, or a straight or branched chain group comprising saturated hydrocarbon groups containing one to one hundred carbon atoms, for example, one to ninety, one to eighty, one to seventy, one to sixty, one to fifty, one to forty carbon atoms, one to thirty carbon atoms, one to twenty carbon atoms, or one to ten carbon atoms, and optionally interrupted with one or more (e.g., 1-15, 1-10, 1-5, 1, 2, 3, 4, 5, 6, 7, 8, 9, or 10) heteroatoms (e.g., selected from O, N, S, P, Se, and B, or selected from N, O, and S), cycloalkyl groups (e.g., a C₃₋₁₀cycloalkyl), heterocycloalkyl groups (e.g., piperazinyl or piperidinyl), aryl groups (e.g., phenyl), or heteroaryl groups (e.g., triazolyl or pyridinyl). Unless otherwise indicated, the chain may be optionally substituted. Substituents can include but are not limited to alkyl, cycloalkyl, alkenyl, cycloalkenyl, alkynyl, heterocycloalkyl, aryl, heteroaryl, hydroxyl, oxo, alkoxy, heteroalkoxy, ester, thioester, carboxy, cyano, nitro, amino, amido, azido, acetamide, and halo (e.g., fluoro, chloro, bromo, or iodo). Linking moieties can be polymer chains, but are not required to be polymeric. Nonlimiting examples of linking moieties include polyalkylene chains (such as polyethylene or polypropylene chains), polyalkylene glycol chains (such as polyethylene glycol and polypropylene glycol), polyamide chains (such as polypeptide chains), and the like. The linking moiety can be attached to the rest of the compound via an amide functional group, an ester functional group, a thioether functional group, an ether functional group, a carbamate functional group, a carbonate functional group, a urea functional group, an alkene functional group, an alkyne functional group, or a heteroaryl ring (e.g., as formed via a Click chemistry reaction between an alkyne and an azide).

As used herein, the term “therapeutically effective amount” means an amount of a compound or combination of therapeutically active compounds (e.g., an opioid receptor modulator or combination of opioid receptor modulators) that ameliorates, attenuates or eliminates one or more symptoms of a particular disease or condition (e.g., heart disease), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

As used herein, the term “pharmaceutically acceptable” means that the referenced substance, such as a compound of the present disclosure, or a formulation containing the compound, or a particular excipient, is safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N+(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. For example, the compounds disclosed herein can be synthesized by solid phase synthesis techniques including those described in Merrifield, J. Am. Chem. Soc. 1963; 85:2149; Davis et al., Biochem. Intl. 1985; 10:394-414; Larsen et al., J. Am. Chem. Soc. 1993; 115:6247; Smith et al., J. Peptide Protein Res. 1994; 44: 183; O'Donnell et al., J. Am. Chem. Soc. 1996; 118:6070; Stewart and Young, Solid Phase Peptide Synthesis, Freeman (1969); Finn et al., The Proteins, 3rd ed., vol. 2, pp. 105-253 (1976); and Erickson et al., The Proteins, 3rd ed., vol. 2, pp. 257-527 (1976). The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

In general, compounds of Formula (I) can be synthesized according to Scheme 1.

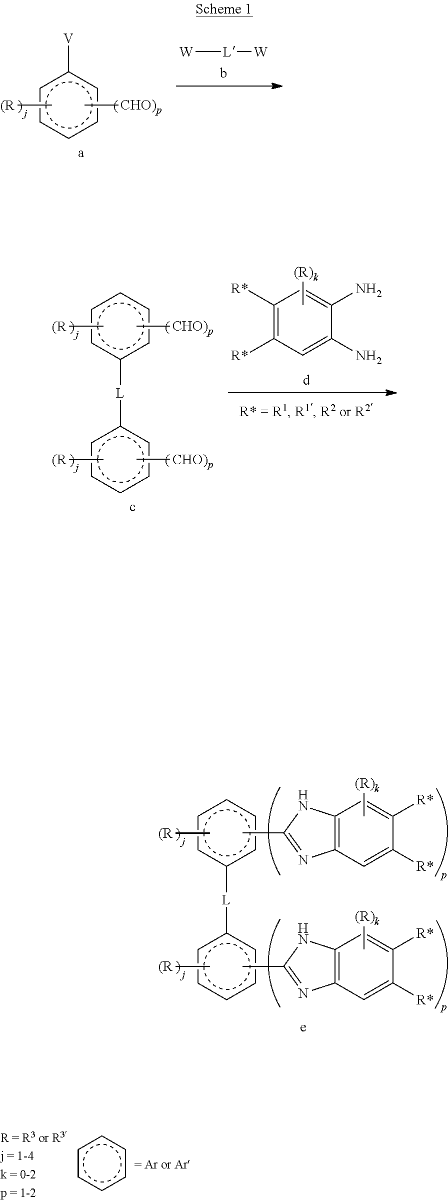

Compounds having structure e can be synthesized using the procedure shown in Scheme 1. Reaction of an optionally substituted mono- or bis-aldehyde a with an optionally-substituted linker precursor b produces dimeric aldehydes having structure c. In compounds a and b, it is understood that V and W represent complementary reactive moieties such that when a and b are reacted, they produce a dimeric aldehyde connected by a linker L, and that L′ represents the portion of a linker L that excludes the reactive moieties W. Subsequent reaction with an o-phenyldiamine compound d gives compounds as described herein, i.e., compounds of Formula I having structure e.

Appropriate reaction conditions for the coupling of compounds a and b can be selected based on the precise nature of compounds a and b, for example, based on the nature of reactive moieties V and W. For example, V can be OH, and W-L′-W can be Br—C₁₋₂₀alkylene-Br, such that a reaction between these species yields a compound c having an L=—O—C₁₋₂₀alkylene-O—, and the reaction conditions to produce said compound c can comprise treatment with a base, e.g., K₂CO₃, in a polar aprotic solvent, e.g., DMF.

Compounds a, b, and d can be purchased commercially or prepared by a variety of methods from commercially-available starting materials. For example, diamines such as compounds having structure d can be prepared by the reduction (e.g., palladium-catalyzed reduction) of appropriate compounds bearing nitro functional groups. Aldehyde compounds having structure a can be prepared by the oxidation (e.g., selective benzylic oxidation with MnO₂) of appropriate compounds bearing alcohol functional groups. The W moieties of a compound b can be prepared by appropriate transformations of other moieties. For example, if the W moieites of a compound b are halogen groups, they can be prepared by reactions known to transform other functional groups into halogen groups, e.g., treatment of hydroxy-bearing compounds (alcohols) with chlorination or bromination reagents (e.g., SOCl₂).

Alternatively, compounds of Formula I can be can be synthesized according to Scheme 2.

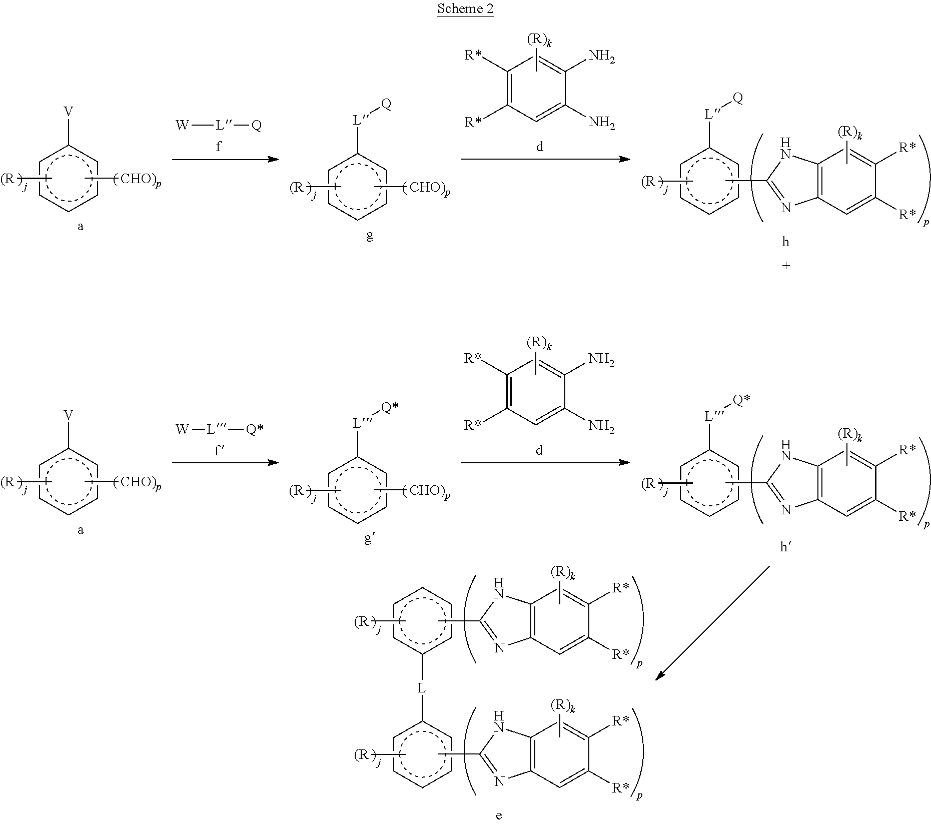

Compounds having structure e can be synthesized using the procedure shown in Scheme 2. For example, mono- or bis-aldehyde a can be reacted with an optionally-substituted linker precursor f or f′ to produce aldehydes having structure g or g′. Linker precursors f and f′ are chosen such that the W moieties are complementary reactive moieties to the V moiety of compound a, as in Scheme 1 above, and such that Q and Q* are also complimentary reactive moieties, and L″ and L′″ represent the portions of a linker L that excludes the reactive moieties W, Q, and Q*. Subsequent reaction with an o-phenyldiamine compound d gives compounds having structures h or h′, each bearing a reactive moiety Q or Q*. A compound h and a compound h′ can coupled under appropriate reaction conditions to provide a dimeric compound as described herein, i.e., compounds of Formula I having structure e.

For example, Q can be azido (—N₃) and Q* can be alkynyl, such that Q and Q* react with each other to form the triazole ring when

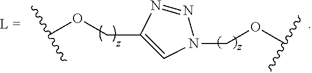

Such a coupling reaction can be carried out under reaction conditions known to a skilled chemist, e.g., under “click” reaction conditions such as a Cu(I)-catalyzed or uncatalyzed cycloaddition conditions.

Compounds f and f′ can be purchased commercially or prepared by a variety of methods from commercially-available starting materials. For example, a compound f or f′ which is an azido compound can be prepared by a substitution reaction between an organic halide, e.g., an organic bromide, and an inorganic azide salt, e.g., sodium azide. Reactions to couple compounds of structure a with compounds f or f′ through reactive moieties V and W can be carried out as described in Scheme 1, above.

Additional synthetic procedures for preparing the compounds disclosed herein can be found in the Examples section.

### Pharmaceutical Formulations, Dosing, and Routes of Administration

Further provided are pharmaceutical formulations comprising a compound as described herein (e.g., compounds of Formula I, Formula Ia, Formula Ib, Formula Ic, Formula II, Formula IIa, Formula IIb, Formula IIc or pharmaceutically acceptable salts of the salts and/or dimers) and a pharmaceutically acceptable excipient.

The compounds described herein can be administered to a subject in a therapeutically effective amount (e.g., in an amount sufficient to prevent or relieve the symptoms of a nucleotide repeat disorder). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein (e.g., compounds of Formula (I)), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent).

The particular carrier employed is limited only by chemico-physical considerations, such as solubility and lack of reactivity with the compound, and by the route of administration.

Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds described herein (e.g., the compounds of Formulas I, Ia, Ib, Ic, II, IIa, IIb, or IIc, and compounds of Table A) can bind nucleic acids. In some embodiments, the compounds bind RNA, e.g., the compounds trigger or inhibit RNA-mediated biological activity, such as gene expression. In various embodiments, the compounds are RNA modulators, e.g., the compounds change, inhibit, or prevent one or more of RNA's biological activities.

The compounds disclosed herein are particularly advantageous for the treatment of diseases or disorders caused by abnormal nucleotide repeats or toxic RNA repeats. The incidence and/or intensity of diseases or disorders associated with expansion of nucleotide repeats in certain genes or introns is reduced.

Toxic RNA is RNA which contains a non-coding nucleotide repeat expansion and which results in cellular damage or disease. In some instances, toxic RNA may accumulate in the nucleus, sequester binding proteins, and result in abnormal splicing for some pre-mRNAs (Osborne et al. Mol. Genet. 15:R162-R169, 2006).

CUG repeat expansions, such as in Myotonic Dystrophy Type 1 (DM1), lead to sequestration of muscleblind-like proteins in ribonuclear foci and depletion in other parts of the nucleoplasm. However, not all toxic RNA diseases are expected to be caused by an identical mechanism. Myotonic dystrophy is the prototypical toxic RNA disease. Spinocerebellar ataxia type 8 (SCA8), Fuchs dystrophy, Huntington Disease (HD), Amyotrophic Lateral Sclerosis (ALS), Frontotemporal Dementia (FTD, also called Frontotemporal Degeneration), and fragile X tremor ataxia syndrome (FXTAS) are representative additional diseases. SCA8 is caused by CUG repeat expansions in a non-coding RNA (ATXN8OS/SCA8), while in FXTAS there is an expansion of about 70-120 CGG repeats in the 5′ UTR of the FMRI gene. Another toxic RNA disease is Huntington disease-like 2 (HDL2), which is caused by a CUG repeat expansion in an intron or the 3′ UTR of the junctophilin 3 gene.

The disclosed methods include methods for treating an expansion repeat disorder, e.g., myotonic dystrophy, comprising administering to a subject a compound that binds a nucleotide repeat expansion in RNA. In some examples, the compound disrupts binding of a protein to the nucleotide repeat expansion. In one example, the method includes use of a compound that disrupts binding of a protein to CUG repeats of DMPK. In a particular example, the compound disrupts binding of a muscleblind-like protein (such as MBNL, MBLL, or MBXL) to CUG repeats of DMPK. In another example, the method includes use of a compound that disrupts binding of a protein to CCUG repeats of ZNF9. In a particular example, the compound disrupts binding of a muscleblind-like protein (such as MBNL, MBLL, or MBXL) to CCUG repeats of ZNF9. The compound may disrupt binding of additional proteins to RNA nucleotide repeat expansions, such as heterogeneous nuclear ribonucleoprotein H (hnRNP H) or other proteins.

Methods for determining whether a compound disrupts RNA binding activity of a protein to a particular sequence are well known to those of skill in the art. See e.g., Current Protocols in Molecular Biology, Ausubel, John Wiley & Sons, 1994; Molecular Cloning, A Laboratory Manual, Sambrook et ah, Cold Spring Harbor Laboratory, 2001. In one example, binding of a protein to a nucleic acid may be determined by EMSA. A nucleic acid sequence (such as a CUG repeat sequence or a CCUG repeat sequence) is incubated in the presence or absence of a protein or mixture of proteins (such as a muscleblind-like protein). The nucleic acid sequence is linked to a detectable label, for example a radioactive, fluorescent, chemiluminescent, or biotin label. The samples are separated by polyacrylamide gel electrophoresis and the labeled nucleic acid is detected, with a shift to slower mobility (higher molecular weight) in the presence of protein compared to nucleic acid in the absence of protein indicating that a protein in the sample binds to the nucleic acid sequence. To determine whether a compound disrupts the protein-nucleic acid binding, samples containing nucleic acid (such as a CUG repeat expansion or a CCUG repeat expansion) and a binding protein (such as a muscleblind-like protein) are incubated in the presence or absence of a candidate compound. If the compound disrupts the protein-nucleic acid interaction, the shift to slower mobility that is observed in the absence of the compound will not occur or will be reduced compared to the shift in the absence of the compound. In another example, RNA-protein interaction may be determined using a filter binding assay. Such assays are well known in the art (see, e.g., Hall and Kranz, in RNA-Protein Interaction Protocols (Methods in Molecular Biology, volume 118), Humana Press, 1999). Briefly, an RNA (such as a CUG repeat sequence or a CCUG repeat sequence) which is labeled (such as with a radioactive label) is incubated in the presence or absence of a protein or mixture of proteins (such as a muscleblind-like protein). The mixture is then passed over a filter (such as a nitrocellulose filter) and RNA-protein complexes are retained and detected.

RNA-protein interaction may also be assessed utilizing fluorescence resonance energy transfer (FRET). Methods for FRET are well known to those of skill in the art. Briefly, a donor fluorophore is coupled to one molecule (such as an RNA binding protein) and an acceptor fluorophore is coupled to another molecule (such as an RNA, for example a CUG or CCUG nucleotide repeat sequence). If the molecules interact, the fluorophores are brought into proximity, such that an excited donor fluorophore can transfer energy to an acceptor fluorophore, which then emits a particular wavelength. Detection of fluorescence emissions may be made utilizing techniques such as fluorescence microscopy or fluorimetry. One of skill in the art will appreciate that the donor and acceptor fluorophores may be coupled to either the protein or RNA binding partners. The disclosed methods include use of compounds that disrupt binding of a protein to an RNA nucleotide repeat expansion. In some cases, the compounds described herein show binding to RNA with an IC₅₀ in a range of about 0.1 to 3000 nM, 0.1 to 1000 nM, 0.1 to 500 nM, 0.1 to 100 nM, 0.1 to 50 nM, 0.1 to 25 nM, 0.1 to 10 nM, 0.1 to 5 nM, 0.1 to 1 nM, 0.1 to 0.5 nM, 1000 to 2000 nM, 100 to 1000 nM, 10 or 100 nM, 5 to 50 nM, or 1 to 10 nM.

Thus, provided herein is a method of modulating RNA in a cell, comprising contacting the cell with a compound or a composition as disclosed herein (e.g., the compounds of formula (I) in an amount sufficient to bind to and modulate RNA. The contacting of the cell can occur in vitro or in vivo. In some cases, contacting of the cell occurs in vitro. In other cases, contacting of the cell occurs in vivo. Therefore, the disclosure includes administering one or more of a compound described herein to a subject, such as a human, in need thereof. In some embodiments, the subject suffers from a nucleotide repeat disorder. Disorders associated with nucleotide repeat expansion (i.e., nucleotide repeat disorders) include, but are not limited to, Myotonic Dystrophy Type 1, Myotonic Dystrophy Type 2, Fuchs dystrophy, Huntington Disease, Amyotrophic Lateral Sclerosis, or Frontotemporal Dementia.

The disclosed methods utilize compounds that bind to RNA nucleotide repeat expansions, such as CUG or CCUG repeats, for treating, e.g., DM. Methods for assessing the usefulness of a compound for treating DM are known to those of skill in the art. For example, compounds may be assessed using models of DM, including cells (such as DM1 or DM2 cells), animal models (such as Drosophila or mouse models of DM), or in human subjects having DM1 or DM2.

A Drosophila model of DM (DM Drosophila) was created by overexpressing a non-coding mRNA containing 480 interrupted CUG repeats of the sequence [(CUG)2OCUCGA]24 (de Haro et al, Hum. Mol. Gen. 15:2138-2145, 2006). DM Drosophila expressing the 480 interrupted CUG repeats exhibit progressive muscle degeneration and nuclear foci containing CUG repeat RNA, unlike flies expressing a (CUG)2O repeat. DM Drosophila expressing the 480 interrupted CUG repeat sequence in the eye also exhibit smaller eyes, disorganization and fusion of the ommatidia, and loss and duplication of the inter-ommatidial bristles. Compounds may be assessed for their ability to decrease or prevent DM-like phenotypes in a Drosophila model of DM. In a particular example, a test compound or mixture is administered orally, such as by mixing with the fly food. Treatment with test compounds may continue for about 1-10 weeks (such as about 1 week to about 4 weeks), or may continue throughout the life of the fly. In one example, a test compound is administered beginning at the larval stage, and continuing throughout the life of the fly. Doses of test compound included in the fly food are from about 1 μM to about 500 μM, such as about 5 μM to about 250 μM, such as about 25 μM to 125 μM.

The phenotype of the DM Drosophila is assessed in the presence and absence of treatment with test compound. In one example, skeletal muscle (for example, indirect flight muscles) of Drosophila may be sectioned for histological examination. The indirect flight muscles of Drosophila expressing the 480 interrupted CUG repeats show vacuolization, loss of muscle fiber organization, and dispersal of nuclei (de Haro et al, 2006). Sections of skeletal muscle from DM Drosophila treated with test compounds are compared to untreated DM Drosophila to determine whether treatment prevents or decreases the muscle degeneration phenotype. In a particular example, muscle degeneration is measured by assessing the size of vacuoles compared to cell size. DM Drosophila are also unable to fly (de Haro et al, 2006), therefore in another example, flies treated with or without test compounds are assessed for their ability to fly and quality or duration of flight. In a further example, Drosophila expressing the 480 interrupted CUG repeat sequence in the eye are treated with test compounds to determine whether treatment prevents or decreases the eye disorganization phenotype.

Drosophila treated with a test compound or compounds are compared to control flies which are not treated with the compound. Control samples are assigned a relative value of 100%. Treatment with the test compound decreases the DM phenotype when the phenotype relative to the control is about 90%, about 80%, about 70%, about 60%, about 50%, about 25%, or about 10%.

A mouse model of DM has been developed by generating mice that express an expanded CUG repeat (250 repeats) in the 3′UTR of a human skeletal actin transgene (Mankodi et al, Science 289:1769-1772, 2000). These mice exhibit myotonia and muscle histopathology consistent with DM (including increases in central nuclei and ring fibers and variability in fiber size), and oxidative muscle fibers. Compounds may be assessed for their ability to decrease or prevent DM-like phenotypes in a mouse model of DM. In a particular example, a test compound or mixture is administered orally, such as by mixing with distilled water. In another example, a test compound or mixture is administered intraperitoneally, intravenously, such as in saline, distilled water, or other appropriate vehicle. In some examples, treatment with test compound may be a single dose or repeated doses. The test compound may be administered about every 6 hours, about every 12 hours, about every 24 hours (daily), about every 48 hours, about every 72 hours, or about weekly. Treatment with repeated doses may continue for about 1 day to about 10 weeks, such as about 2 days, about 3 days, about 4 days, about 5 days, about 6 days, about 1 week, about 2 weeks, about 4 weeks, about six weeks, about 8 weeks, or about 10 weeks. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 40 mg/kg. In one example, myotonic discharges of skeletal muscle (such as forelimb muscles or paraspinal muscles) are assessed by electromyography (EMG). EMG recordings are made from DM mice treated with or without test compounds and the number of mice exhibiting myotonic discharges in each group is compared to determine if the test compound inhibits or decreases myotonia in a mouse model of DM. In another example, skeletal muscle histology may be compared between DM mice treated with or without test compound to assess if the test compound inhibits or decreases the muscle pathology of the DM mouse model. In a further example, features of oxidative muscle fibers, such as upregulation of succinate dehydrogenase and cytochrome oxidase are compared between DM mice treated with or without test compounds. In another example, rescue of mis-splicing of RNA (such as cTNT, IR, Clc-I or Sercal) in mice treated with the test compound is assessed.

Mice treated with a test compound or compounds are compared to control mice which are not treated with the compound. Control samples are assigned a relative value of 100%. Treatment with the test compound decreases the DM phenotype when the phenotype relative to the control is about 90%, about 80%, about 70%, about 60%, about 50%, about 25%, or about 10%.

The compounds described herein can be used to decrease or prevent DM phenotypes in human subjects with DM1 or DM2. In a particular example, a compound or mixture is administered orally, such as by mixing with distilled water. In another example, a test compound or mixture is administered intravenously, such as in saline or distilled water. In some examples, treatment with test compound may be a single dose or repeated doses. The test compound may be administered about every 6 hours, about every 12 hours, about every 24 hours (daily), about every 48 hours, about every 72 hours, or about weekly. Treatment with repeated doses may continue for a period of time, for example for about 1 week to 12 months, such as about 1 week to about 6 months, or about 2 weeks to about 3 months, or about 1 to 2 months. Administration of a compound may also continue indefinitely. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 50 mg/kg.

Methods of assessing DM phenotypes are well known to those of skill in the art. DM phenotypes in affected individuals include muscle weakness (which may lead to foot drop and gait disturbance, as well as difficulty in performing tasks requiring hand dexterity), myotonia (sustained muscle contraction), which often manifests as the inability to quickly release a hand grip (grip myotonia) and which can be demonstrated by tapping a muscle with a reflex hammer (percussion myotonia), and myotonic discharges observed by EMG recording. Pathologic features may be observed by muscle biopsy, including rows of internal nuclei, ring fibers, sarcoplasmic masses, type I fiber atrophy, and increased number of intrafusal muscle fibers.

Changes in DM phenotypes may be monitored in DM subjects following administration of one or more compounds disclosed herein. DM phenotypes may be compared to DM subjects who have not received the compounds or comparison may be made to the subject's phenotype prior to administration of the compound in order to assess effectiveness of the compound for treatment of DM.

It will be understood that the methods and compositions described herein for treating DM, comprising administering a compound that binds an RNA nucleotide repeat expansion, are applicable to methods of treating toxic RNA diseases, such as those described above. The methods for assessing the effectiveness of test compounds for treating such diseases in cells, appropriate animal models, or affected subjects are known to one of skill in the art. For example, animal models of FXTAS (Jin et al, Neuron 39:739-747, 2003; Brouwer et al, Exp. Cell Res. 313:244-253, 2007) and SCA8 (Mutsuddi et al., Curr. Biol. 14:302-308, 2004; Moseley et al., Nature Genet. 38:758-769, 2006) are known to those in the art.

Uses of the compounds disclosed herein in the preparation of a medicament for treating nucleotide repeat disorders also are provided herein.

The disclosure herein will be understood more readily by reference to the following examples, below.

### EXAMPLES

The following examples are provided for illustration and are not intended to limit the scope of the disclosure.

### Synthetic Procedures for Compounds of Formula (I)

General Experimental Procedures.

All reagents and solvents were obtained from commercial sources and used without additional purification. The synthesis of certain precursors is detailed in the Preparations, below. The syntheses of compounds of Formula I and Formula II are detailed in the Examples, below.

### Example 1: Synthesis of 3,3′-((2,2′-(2-(2-methoxyethoxy)-1,4-phenylene)bis(1H-benzo[d]imidazole-6,2-diyl))bis(oxy))bis(N,N-dimethylpropan-1-amine)

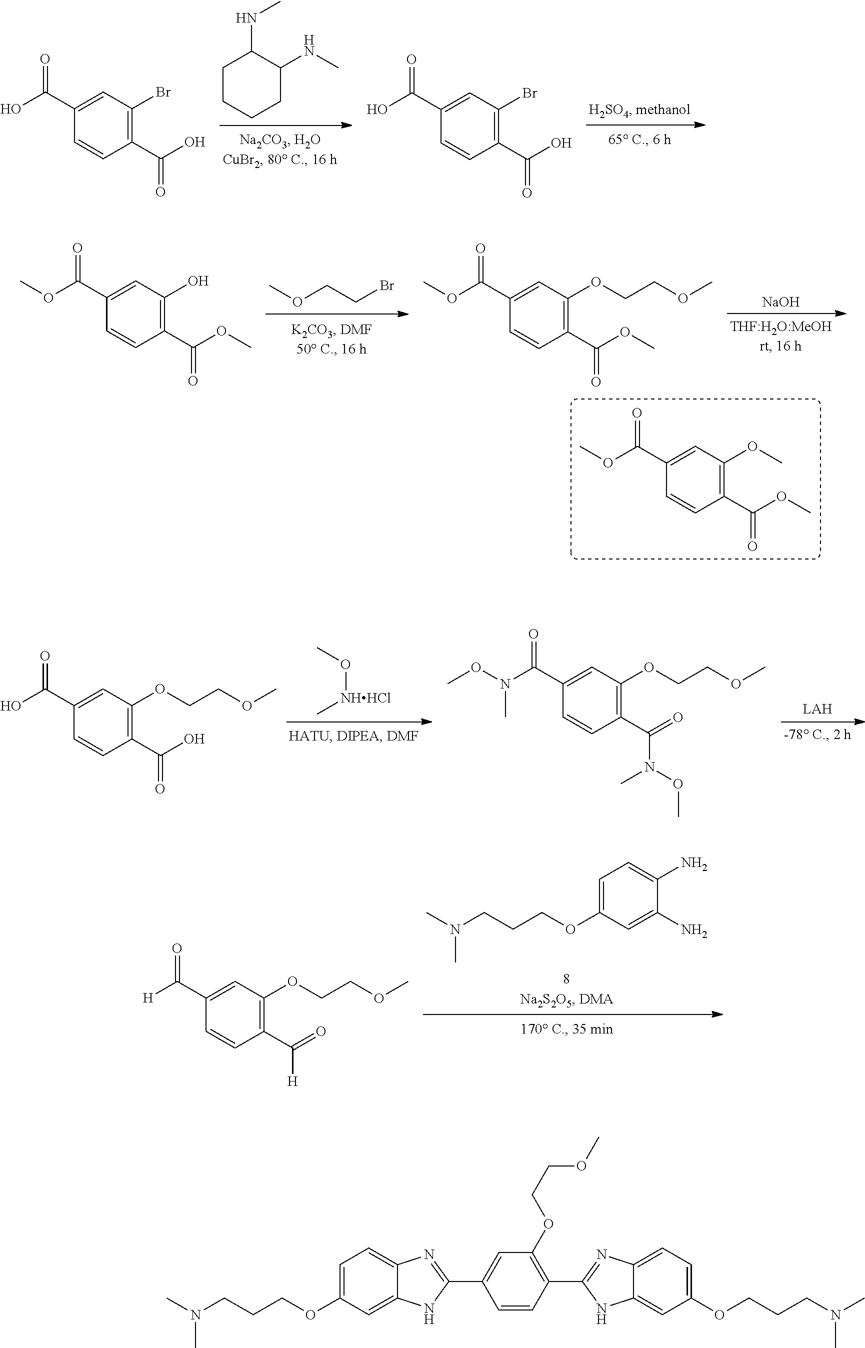

### Synthesis of 2-hydroxyterephthalic Acid

To a suspension of 2-bromoterephthalic acid (25.0 g, 102 mmol) in water (120 mL) at room temperature under N₂ atmosphere, was added Na₂CO₃ (21.6 g, 204 mmol) and the reaction mixture was heated at 80° C. for 1 h. After 1 h, a solution of N1,N2-dimethylcyclohexane-1,2-diamine (0.29 g, 2.04 mmol) and copper(II) bromide (0.22 g, 1.02 mmol) in water (10.0 mL) was added to the above reaction mixture, and the mixture was heated at 80° C. for 16 h. The reaction mixture was cooled to room temperature. and poured onto 2N aqueous HCl (20.0 mL) and stirred for 15 min, whereupon an off-white solid precipitated. The off white solid was collected by filtration under vacuum and washed with water (2×100 mL) to get 2-hydroxyterephthalic acid.

### Synthesis of dimethyl 2-hydroxyterephthalate

To a solution of 2-hydroxyterephthalic acid (14.0 g, 77.0 mmol) in MeOH (100 mL) at 0° C., was added conc.H₂SO₄ (15.0 mL) and the reaction mixture was heated to reflux for 5 h.

The reaction mixture was cooled to room temperature and neutralized with satd. aqueous NaHCO₃ (50.0 mL) and extracted with EtOAc (3×100 mL). The combined organic extracts were washed with brine (100 mL), dried (Na₂SO₄), filtered and concentrated under vacuum to obtain dimethyl 2-hydroxyterephthalate.

### Synthesis of dimethyl 2-(2-methoxyethoxy)terephthalate

To a stirred solution of dimethyl 2-hydroxyterephthalate (1.30 g, 3.57 mmol) in DMF (8.00 mL) was added K₂CO₃ (0.98 g, 7.14 mmol) followed with 1-bromo-2-methoxyethane (8.69 g, 5.35 mmol) and the reaction mixture was heated at 40° C. for 16 h. The reaction mixture was cooled to room temperature and the contents were poured on to ice cold water, and the aqueous phase was extracted with EtOAc (2×100 mL). The combined EtOAc extracts were washed with brine (50 mL), dried over anhydrous Na₂SO₄, filtered and concentrated under vacuum to obtain the crude product. The product was purified by silica gel chromatography using EtOAC: hexane (3:7). The fractions containing the product were combined and concentrated under vacuum to obtain dimethyl 2-(2-methoxyethoxy)terephthalate.

Also isolated dimethyl 2-methoxyterephthalate.

### Synthesis 2-(2-methoxyethoxy)terephthalic Acid

To a stirred solution of dimethyl 2-(2-methoxyethoxy)terephthalate (0.40 g, 1.49 mmol) in THF (8.00 mL), H₂O (5.00 mL), and MeOH (3.00 mL), was added NaOH (0.29 g, 7.46 mmol), at room temperature and the reaction mixture was stirred at room temperature for 12 h.

The reaction mixture was concentrated under reduced pressure to obtain a residue. The residue was diluted with H₂O (10.0 mL) and acidified to pH 2, using 2N aq.HCl, whereupon a brown solid precipitated. The precipitate was filtered under vacuum and washed with water (10 mL), and dried under vacuum to obtain 2-(2-methoxyethoxy)terephthalic acid. The crude product was directly used in the next step.

### Synthesis of N1,N4-dimethoxy-2-(2-methoxyethoxy)-N1,N4-dimethylterephthalamide

To a suspension of 2-(2-methoxyethoxy)terephthalic acid (0.33 g, 1.34 mmol) in DMF (10.0 mL) at 0° C. under N₂ atmosphere, was added HATU (2.06 g, 5.52 mmol), DIPEA (1.07 g, 8.24 mmol) and N,O-dimethylhydroxylamine hydrochloride (0.40 g, 5.52 mmol), and the reaction mixture was stirred at room temperature for 2 h. The reaction mixture was poured on to ice cold water, and the aqueous phase was extracted with EtOAc (2×100 mL). The combined EtOAc extracts were washed with brine (50 mL), dried over anhydrous Na₂SO₄, filtered and concentrated under vacuum to obtain the crude product. The product was purified by silica gel chromatography using EtOAC: hexane (3:7). The fractions containing the product were combined and concentrated under vacuum to obtain N1,N4-dimethoxy-2-(2-methoxyethoxy)-N1,N4-dimethylterephthalamide.

### Synthesis of 2-(2-methoxyethoxy)terephthalaldehyde

To a solution of N1,N4-dimethoxy-2-(2-methoxyethoxy)-N1,N4-dimethylterephthalamide (0.40 g, 1.26 mmol) in THF (20.0 mL) under argon atmosphere, was added LiAIH₄ (2.0 M in THF, 1.80 mL, 3.68 mmol) at −78° C. in a dropwise manner over a period of 10 min. The reaction mixture was allowed to gradually reach r. t and stirred for 1 h. The reaction mixture was cooled to 0° C. and quenched with saturated aqueous Na₂SO₄ solution.

The precipitated solids were filtered under vacuum, washed with 10% MeOH in CH₂Cl₂ (100 mL). The organic layer was washed with brine (20 mL) and concentrated under reduced pressure to obtain the crude product. The product was purified by silica gel chromatography using EtOAC: hexane (2:8). The fractions containing the product were combined and concentrated under vacuum to obtain 2-(2-methoxyethoxy)terephthalaldehyde.

### Synthesis of 3,3′-((2,2′-(2-(2-methoxyethoxy)-1,4-phenylene)bis(1H-benzo[d]imidazole-6,2-diyl))bis(oxy))bis(N,N-dimethylpropan-1-amine)

In a microwave vial, a mixture of 2-(2-methoxyethoxy)terephthalaldehyde (0.10 g, 0.48 mmol), 4-(3-(dimethylamino)propoxy)benzene-1,2-diamine (0.20 g, 0.96 mmol) and Na₂S₂O₅ (0.83 g, 0.96 mmol) in DMA (3.00 mL) was heated at 170° C. for 40 min. The reaction mixture was cooled to room temperature and the contents were slowly poured into water (50 mL) and stirred for 10 min, whereupon, a brown solid precipitated. The brown solid was filtered under vacuum and washed with water (2×50 mL), and dried to obtain the crude product. The product was purified by mass triggered HPLC. The fractions containing only the pure product were combined for concentration to obtain the title compound. MS (ESI): m/z=587 [M+H]⁺.

### Example 2: Synthesis of 2-(4-(6-(3-(Dimethylamino) propoxy)-1H-benzo[d]imidazol-2-yl)-2-(2-hydroxyethoxy) phenyl)-1H-benzo[d]imidazol-6-ol

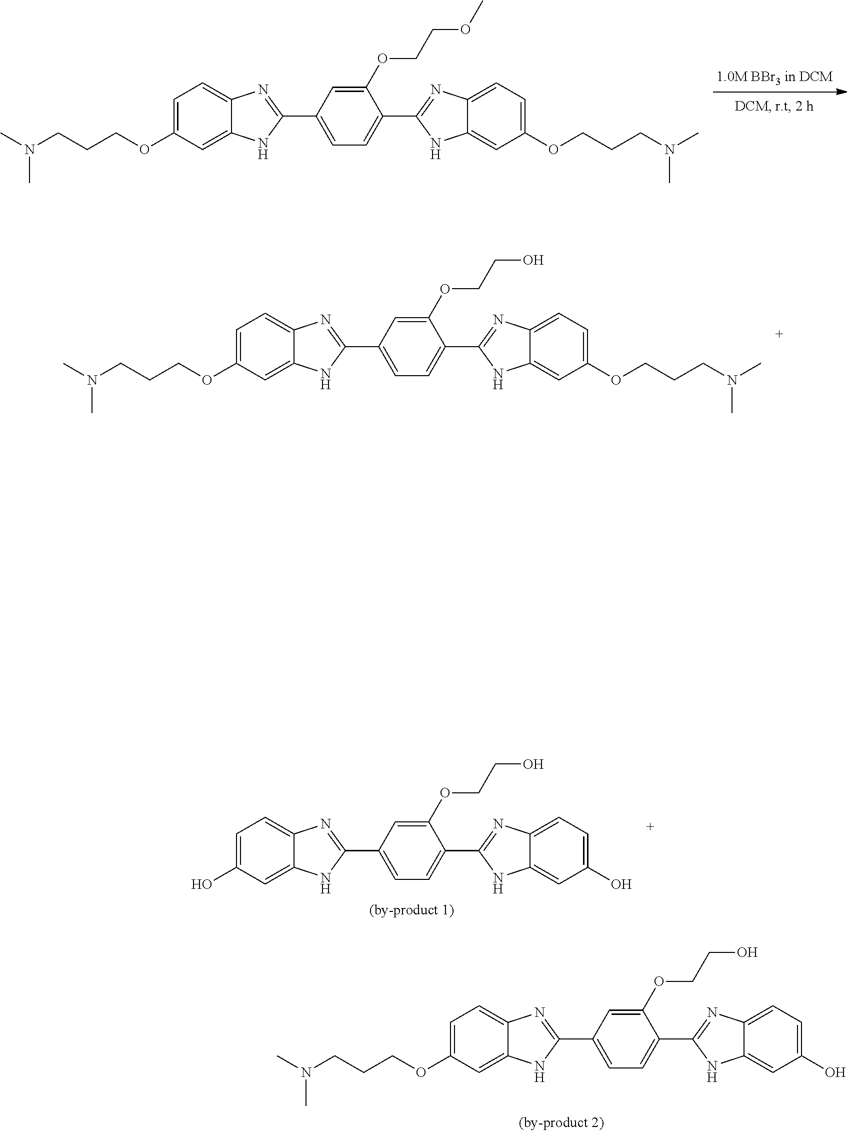

### Synthesis of 2-(2,5-bis(6-(3-(dimethylamino)propoxy)-1H-benzo[d]imidazol-2-yl)phenoxy)ethanol

To a solution of 3,3′-((2,2′-(2-(2-methoxyethoxy)-1,4-phenylene)bis(1H-benzo[d]imidazole-6,2-diyl))bis(oxy))bis(N,N-dimethylpropan-1-amine (200 mg, 0.34 mmol) in CH₂Cl₂ (15.0 mL) at 0° C. under argon atmosphere, was added BBr₃ (2.0 M in CH₂Cl₂, 1.20 mL, 1.02 mmol) in a dropwise manner over a period of 10 min. After completen addition, the mixture was stirred at room temperature for 1 h, neutralized with methanol (5.0 mL), and concentrated under reduced pressure to obtain the crude product. The product was purified by mass triggered HPLC. Fractions containing only the pure product were combined for concentration to obtain the title compound. Also isolated 2, 2′-(2-(2-hydroxyethoxy)-1,4-phenylene)bis(1H-benzo[d]imidazol-6-ol) (by-product 1), and 2-(4-(6-(3-(dimethylamino) propoxy)-1H-benzo[d]imidazol-2-yl)-2-(2-hydroxyethoxy) phenyl)-1H-benzo[d]imidazol-6-ol (by-product 2)

2-(2,5-bis(6-(3-(dimethylamino)propoxy)-1H-benzo[d]imidazol-2-yl)phenoxy)ethanol: MS (ESI): m/z=573 [M+H]+.

2, 2′-(2-(2-Hydroxyethoxy)-1,4-phenylene)bis(1H-benzo[d]imidazol-6-ol): MS (ESI): m/z=403 [M+H]+.

2-(4-(6-(3-(Dimethylamino) propoxy)-1H-benzo[d]imidazol-2-yl)-2-(2-hydroxyethoxy) phenyl)-1H-benzo[d]imidazol-6-ol: MS (ESI): m/z=488 [M+H]+.

### Example 3: Synthesis of 3,3′-((2,2′-(2-(prop-2-yn-1-yloxy)-1,4-phenylene)bis(1H-benzo[d]imidazole-6,2-diyl))bis(oxy))bis(N,N-dimethylpropan-1-amine))

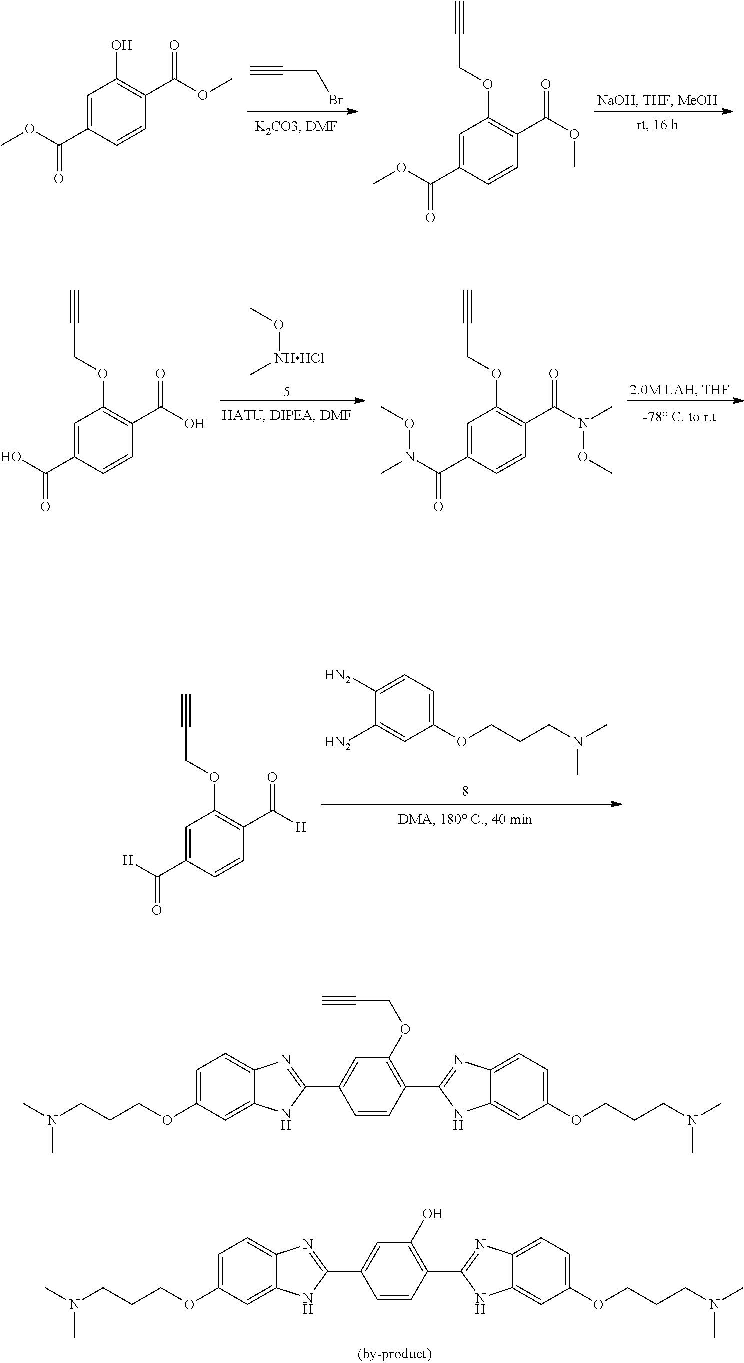

### Synthesis of dimethyl 2-(prop-2-yn-1-yloxy)terephthalate

To a stirred solution of dimethyl 2-hydroxyterephthalate (0.75 g, 3.57 mmol) in DMF (8.00 mL) was added K₂CO₃ (0.98 g, 7.14 mmol) followed with 3-bromoprop-1-yn (8.69 g, 5.35 mmol) and the reaction mixture was stirred at room temperature for 16 h. The reaction mixture was cooled to room temperature and the contents were poured on to ice cold water (30 mL), and extracted with EtOAc (2×50 mL). The combined EtOAc extracts were washed with brine (50 mL), dried over anhydrous Na₂SO₄, filtered and concentrated under vacuum to obtain the crude product. The product was purified by silica gel chromatography using CH₃OH: CH₂Cl₂ (1:9). The fractions containing the product were combined and concentrated under vacuum to obtain dimethyl 2-(prop-2-yn-1-yloxy)terephthalate.

### Synthesis of 2-(prop-2-yn-1-yloxy)terephthalic Acid

To a stirred solution of dimethyl 2-(prop-2-yn-1-yloxy)terephthalate (0.70 g, 2.82 mmol) in THF (10.0 mL), H₂O (6.00 mL), and MeOH (3.00 mL), was added NaOH (0.56 g, 14.10 mmol), at room temperature and the reaction mixture was heated at 90° C. for 2 h. The reaction mixture was concentrated under reduced pressure to remove THF, and the remaining contents were acidified to pH 2 using 2N aqueous HCl, whereupon a brown solid precipitated. The solid was filtered under vacuum, washed with water (10 mL), and dried under vacuum to obtain 2-(prop-2-yn-1-yloxy)terephthalic acid. The crude product was directly used in the next step.

### Synthesis of N1,N4-dimethoxy-N1,N4-dimethyl-2-(prop-2-yn-1-yloxy)terephthalamide

To a suspension of 2-(prop-2-yn-1-yloxy)terephthalic acid (0.50 g, 2.27 mmol, crude) in DMF (10.0 mL), at 0° C. under N₂ atmosphere, was added HATU (2.16 g, 5.68 mmol), DIPEA (1.47 g, 11.3 mmol) and N,O-dimethylhydroxylamine hydrochloride (0.35 g, 5.68 mmol), and the reaction mixture was stirred at room temperature for 2 h. The reaction mixture was poured on to ice cold water, and the aqueous phase was extracted with EtOAc (2×50 mL). The combined EtOAc extracts were washed with brine (50 mL), dried over anhydrous Na₂SO₄, filtered and concentrated under vacuum to obtain the crude product. The product was purified by silica gel chromatography using EtOAC: hexanes (3:7). The fractions containing the product were combined and concentrated under vacuum to obtain N1,N4-dimethoxy-N1,N4-dimethyl-2-(prop-2-yn-1-yloxy)terephthalamide

### Synthesis of 2-(prop-2-yn-1-yloxy)terephthalaldehyde

To a solution of N1,N4-dimethoxy-N1,N4-dimethyl-2-(prop-2-yn-1-yloxy)terephthalamide (0.35 g, 1.14 mmol) in THF (40.0 mL), under argon atmosphere at −78° C., was added LiAIH₄ (2.0 M in THF, 0.26 g, 6.86 mmol) in a dropwise manner over a period of 10 min. The reaction mixture was allowed to stir at room temperature for 2 h. The reaction mixture was cooled to 0° C. and quenched with satd. aqueous Na₂SO₄ solution, whereupon white solids precipitated. The precipitated solids were filtered under vacuum and washed with 10% MeOH in DCM (2×50 mL). The organic layer was washed with brine (20 mL), dried over anhydrous Na₂SO₄, filtered and concentrated under reduced pressure to obtain the crude product. The product was purified by silica gel chromatography using EtOAC: hexanes (2:8). The fractions containing the product were combined and concentrated under vacuum to obtain 2-(prop-2-yn-1-yloxy) terephthalaldehyde

### Synthesis of 3,3′-((2,2′-(2-(prop-2-yn-1-yloxy)-1,4-phenylene)bis(1H-benzo[d]imidazole-6,2-diyl))bis(oxy))bis(N,N-dimethylpropan-1-amine)

In a microwave vial, a mixture of 2-(prop-2-yn-1-yloxy)terephthalaldehyde (0.13 g, 0.69 mmol), 4-(3-(dimethylamino)propoxy)benzene-1,2-diamine (0.29 g, 1.38 mmol) and Na₂S₂O₅ (0.13 g, 0.69 mmol) in DMA (3.00 mL) was heated at 170° C. for 40 min. The reaction mixture was cooled to room temperature and the contents were slowly poured into water (50 mL) and stirred for 10 min, whereupon, a brown solid precipitated. The brown solid was filtered under vacuum and washed with water (2×50 mL), and dried to obtain the crude product. The product was purified by mass triggered HPLC. The fractions containing only the pure product were combined for concentration to obtain the title compound, and 2,5-bis(6-(3-(dimethylamino)propoxy)-1H-benzo[d]imidazol-2-yl)phenol. MS (ESI): m/z=567 [M+H]⁺. (by-product): MS (ESI): m/z=529 [M+H]⁺.

### Example 4: Synthesis of 3,3′-((2,2′-(2-(2-azidoethoxy)-1,4-phenylene)bis(1H-benzo[d]imidazole-6,2-diyl))bis(oxy))bis(N,N-dimethylpropan-1-amine)

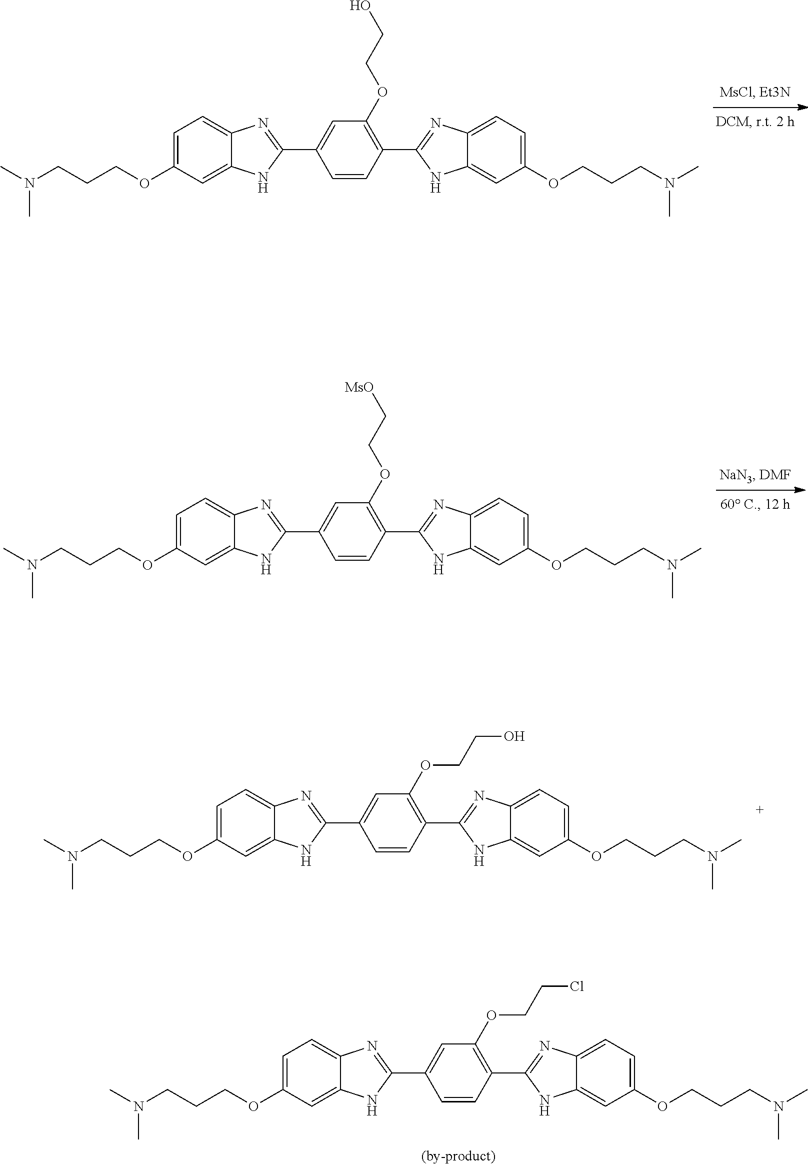

### Synthesis of 2-(2,5-bis(6-(3-(dimethylamino)propoxy)-1H-benzo[d]imidazol-2-yl)phenoxy)ethyl methanesulfonate

To a stirred solution of 2-(2,5-bis(6-(3-(dimethylamino)propoxy)-1H-benzo[d]imidazol-2-yl)phenoxy)ethanol (100 mg, 0.17 mmol) in anhydrous DCM (10.0 mL), at 0° C. under N₂ atmosphere, was added Et₃N (0.13 mL, 0.87 mmol) followed with methane sulfonyl chloride (30.0 mg, 0.26 mmol) in a dropwise manner. After complete addition, the reaction mixture was stirred at room temperature for 2 h. The reaction mixture was concentrated under vacuum to obtain 2-(2,5-bis(6-(3-(dimethylamino)propoxy)-1H-benzo[d]imidazol-2-yl)phenoxy)ethyl methanesulfonate The crude product was used directly in the next step.

### Synthesis of 3,3′-((2,2′-(2-(2-azidoethoxy)-1,4-phenylene)bis(1H-benzo[d]imidazole-6,2-diyl))bis(oxy))bis(N,N-dimethylpropan-1-amine)

To a stirred solution of 3-((2-(4-(5-(3-(dimethylamino)propoxy)-1H-benzo[d]imidazol-2-yl)phenyl)-1H-benzo[d]imidazol-5-yl)oxy)propyl methane sulfonate (110 mg, 0.16 mmol) in anhydrous DMF (6 mL), at room temperature under N₂ atmosphere, was added sodium azide (55 mg, 0.84 mmol), and the reaction mixture was stirred at 60° C. for 12 h. The reaction mixture was cooled to room temperature and the contents were slowly poured into ice-cold water and stirred for 10 min, whereupon a solid precipitated. The solid was collected by filtration under vacuum, washed with water (10 mL) and dried to obtain the crude product. The product was purified by mass triggered HPLC. The fractions containing only the pure product were combined for concentration to obtain the title compound, and 3,3′-((2,2′-(2-(2-chloroethoxy)-1,4-phenylene)bis(1H-benzo[d]imidazole-6,2-diyl))bis(oxy))bis(N,N-dimethylpropan-1-amine) (by-product). MS (ESI): m/z=598 [M+H]+. By-product MS (ESI): m/z=591 [M+H]⁺.

### Example 5: Synthesis of 3,3′-((2,2′-(2-(but-3-yn-1-yloxy)-1,4-phenylene)bis(1H-benzo[d]imidazole-6,2-diyl))bis(oxy))bis(N,N-dimethylpropan-1-amine)

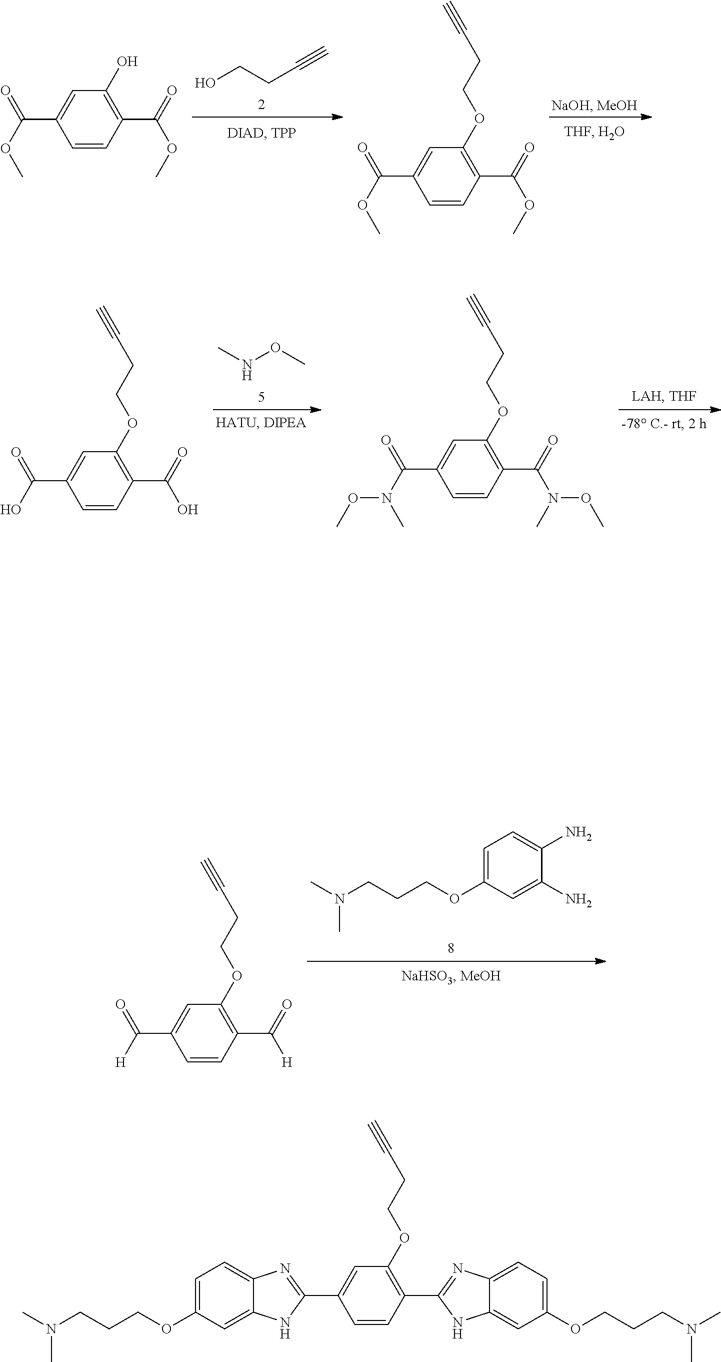

### Synthesis of dimethyl 2-(but-3-yn-1-yloxy)terephthalate

To a stirred solution of dimethyl 2-hydroxyterephthalate (1.00 g, 4.76 mmol) in anhydrous THF (15.0 mL) at 0° C. under N₂ atmosphere, was added but-3-yn-1-ol (0.67 g, 9.52 mmol), PPh₃ (1.87 g, 7.14 mmol) and DIAD (1.44 g, 7.14 mmol) and the reaction mixture was stirred at room temperature for 16 h. The reaction mixture was poured on to ice cold water (30 mL), and extracted with EtOAc (2×50 mL). The combined EtOAc extracts were washed with brine (50 mL), dried over anhydrous Na₂SO₄, filtered and concentrated under vacuum to obtain the crude product. The product was purified by silica gel chromatography using EtOAc: Hexanes (6:4). The fractions containing the product were combined and concentrated under vacuum to obtain dimethyl 2-(but-3-yn-1-yloxy)terephthalate.

### Synthesis of 2 2-(but-3-yn-1-yloxy)terephthalic Acid

To a stirred solution of dimethyl 2-(but-3-yn-1-yloxy)terephthalate (1.00 g, 3.81 mmol) in THF (10.0 mL), H₂O (6.00 mL), and MeOH (4.00 mL), was added NaOH (0.61 g, 15.25 mmol), at room temperature and the reaction mixture was stirred for 3 h. The reaction mixture was concentrated under reduced pressure to remove THF, and the remaining contents were acidified to pH 2 using 2N aqueous HCl, whereupon a brown solid precipitated. The solid was filtered under vacuum, washed with water (10 mL), and dried under vacuum to obtain 2-(but-3-yn-1-yloxy)terephthalic acid. The crude product was directly used in the next step.

### Synthesis of 2-(but-3-yn-1-yloxy)-N1,N4-dimethoxy-N1,N4-dimethylterephthalamide

To a suspension of 2-(but-3-yn-1-yloxy)terephthalic acid (0.50 g, 2.27 mmol, crude) in anhydrous DMF (10.0 mL), at 0° C. under N₂ atmosphere, was added HATU (2.16 g, 5.68 mmol), DIPEA (1.47 g, 11.3 mmol) followed with N,O-dimethylhydroxylamine hydrochloride (0.35 g, 5.68 mmol), and the reaction mixture was stirred at room temperature for 2 h. The reaction mixture was poured on to ice cold water, and the aqueous phase was extracted with EtOAc (2×50 mL). The combined EtOAc extracts were washed with brine (50 mL), dried over anhydrous Na₂SO₄, filtered and concentrated under vacuum to obtain the crude product. The product was purified by silica gel chromatography using EtOAC: hexanes (3:7). The fractions containing the product were combined and concentrated under vacuum to obtain 2-(but-3-yn-1-yloxy)-N1,N4-dimethoxy-N1,N4-dimethylterephthalamide

### (Synthesis of 2-(but-3-yn-1-yloxy)terephthalaldehyde

To a solution of 2-(but-3-yn-1-yloxy)-N1,N4-dimethoxy-N1,N4-dimethylterephthalamide (0.30 g, 0.93 mmol) in anhydrous THF (20.0 mL), at −78° C. under argon atmosphere, was added LiAIH₄ (2.0 M in THF, 0.21 g, 2.80 mmol) in a dropwise manner over a period of 10 min. After complete addition, the mixture was stirred at room temperature for 2 h. The reaction mixture was cooled to 0° C. and quenched with satd. aqueous Na₂SO₄ solution, whereupon white solids precipitated. The precipitated solids were filtered under vacuum and washed with 10% MeOH in DCM (2×50 mL). The organic layer was washed with brine (20 mL), dried over anhydrous Na₂SO₄, filtered and concentrated under reduced pressure to obtain the crude product. The product was purified by silica gel chromatography using EtOAC: hexanes (4:6). The fractions containing the product were combined and concentrated under vacuum to obtain 2-(but-3-yn-1-yloxy)terephthalaldehyde

### Synthesis of 3,3′-((2,2′-(2-(but-3-yn-1-yloxy)-1,4-phenylene)bis(1H-benzo[d]imidazole-6,2-diyl))bis(oxy))bis(N,N-dimethylpropan-1-amine)

To a stirred solution of 2-(but-3-yn-1-yloxy)terephthalaldehyde (0.10 g, 0.54 mmol) in CH₃OH (15.0 mL) at room temperature under N₂ atmosphere, was added NaHSO₃ (0.23 g, 2.72 mmol) followed with 4-(3-(dimethylamino)propoxy)benzene-1,2-diamine (0.57 g, 2.72 mmol) and the reaction mixture was stirred at 80° C. for 16 h. The reaction mixture was concentrated under reduced pressure to obtain crude product. The product was purified by mass triggered HPLC. The fractions containing only the pure product were combined for concentration to obtain the title compound. MS (ESI): m/z=581 [M+H]⁺.

### Example 6: Synthesis of 3,3′-((2,2′-(2-(3-azidopropoxy)-1,4-phenylene)bis(1H-benzo[d]imidazole-6,2-diyl))bis(oxy))bis(N,N-dimethylpropan-1-amine))

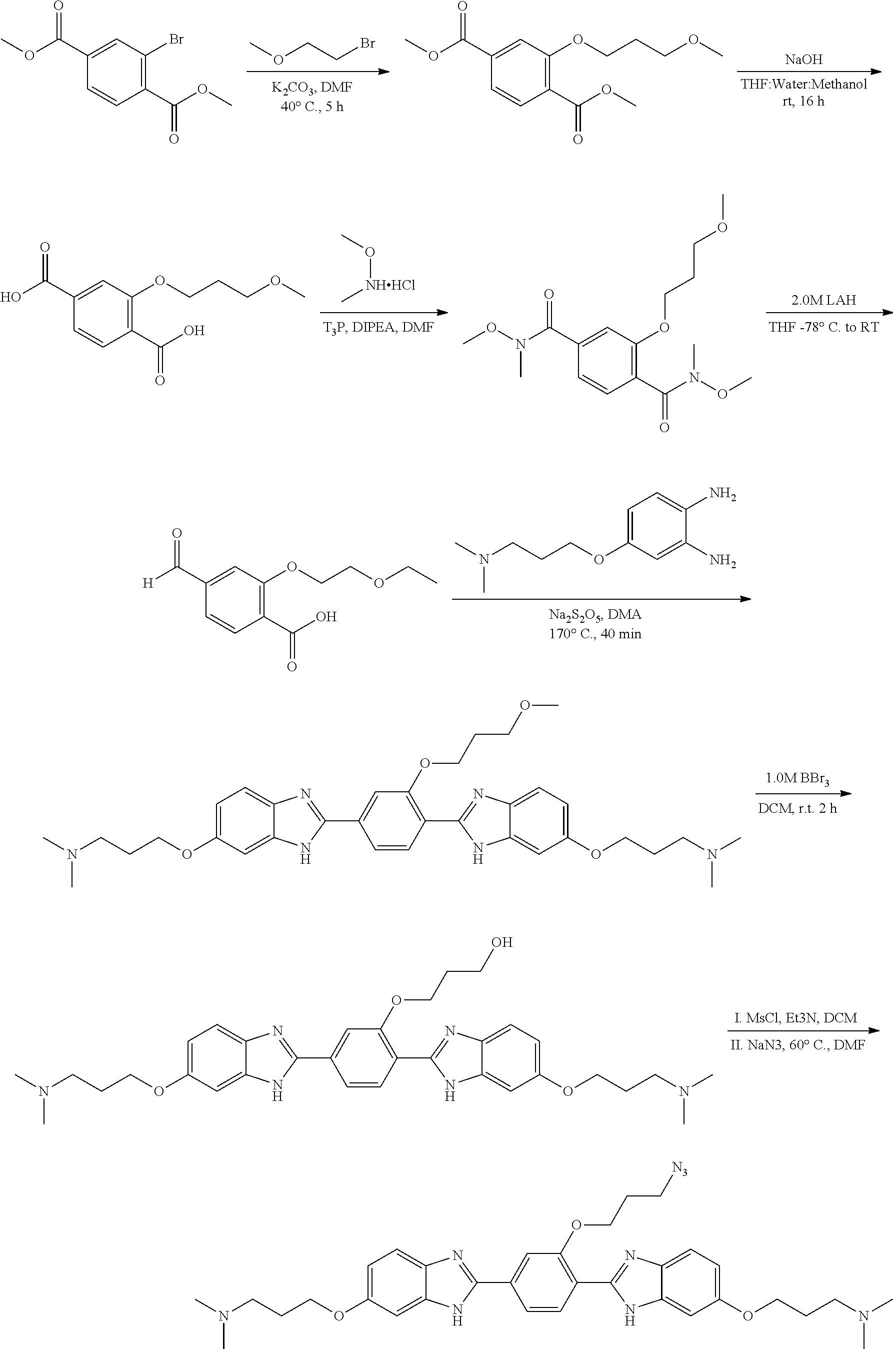

### Synthesis of dimethyl 2-(3-methoxypropoxy)terephthalate

To a stirred solution of dimethyl 2-hydroxyterephthalate (1.00 g, 4.76 mmol) in anhydrous DMF (12.0 mL) was added K₂CO₃ (1.97 g, 14.7 mmol) followed with 1-bromo-3-methoxypropane (1.09 g, 7.14 mmol) and the reaction mixture was heated at 40° C. for 6 h.

The reaction mixture was cooled to room temperature and the contents were poured on to ice cold water, and the aqueous phase was extracted with EtOAc (2×100 mL). The combined EtOAc extracts were washed with brine (50 mL), dried over anhydrous Na₂SO₄, filtered and concentrated under vacuum to obtain the crude product. The product was purified by silica gel chromatography using EtOAc: Hexanes (3:7). The fractions containing the product were combined and concentrated under vacuum to obtain dimethyl 2-(3-methoxypropoxy) terephthalate

### Synthesis 2-(3-methoxypropoxy)terephthalic Acid

To a stirred solution of dimethyl 2-(3-methoxypropoxy) terephthalate (1.00 g, 3.54 mmol) in THF (15.0 mL), H₂O (6.00 mL), and MeOH (5.00 mL), was added NaOH (0.70 g, 17.7 mmol), and the reaction mixture was stirred at room temperature for 4 h. The reaction mixture was concentrated under reduced pressure to obtain a residue. The residue was diluted with H₂O (10.0 mL) and acidified to pH 2, using 2N aq.HCl, whereupon a white solid precipitated.

The precipitate was filtered under vacuum and washed with water (10 mL), and dried under vacuum to obtain 2-(3-methoxypropoxy)terephthalic acid. The crude product was directly used in the next step.

### Synthesis of N1,N4-dimethoxy-2-(3-methoxypropoxy)-N1,N4-dimethylterephthalamide

To a suspension of 2-(3-methoxypropoxy)terephthalic acid (0.85 g, 3.34 mmol) in anhydrous DMF (10.0 mL) at 0° C. under N₂ atmosphere, was added T₃P (4.26 g, 13.3 mmol), DIPEA (3.5 mL, 20.4 mmol) followed with N,O-dimethyl hydroxylamine hydrochloride (0.97 mg, 10.0 mmol), and the reaction mixture was stirred at room temperature for 2 h. The reaction mixture was poured on to ice cold water, and the aqueous phase was extracted with EtOAc (2×100 mL). The combined EtOAc extracts were washed with brine (50 mL), dried over anhydrous Na₂SO₄, filtered and concentrated under vacuum to obtain the crude product. The product was purified by silica gel chromatography using EtOAc: Hexanes (3:7). The fractions containing the product were combined and concentrated under vacuum to obtain N1,N4-dimethoxy-2-(3-methoxypropoxy)-N1,N4-dimethylterephthalamide

### Synthesis of 2-(3-methoxypropoxy)terephthalaldehyde

To a solution of N1,N4-dimethoxy-2-(3-methoxypropoxy)-N1,N4-dimethylterephthalamide (0.75 g, 2.26 mmol) in anhydrous THF (30 mL) at −78° C. under argon atmosphere, was added LiAIH₄ (2.0 M solution in THF, 4.4 mL, 8.88 mmol) in a dropwise manner over a period of 10 min. The reaction mixture was gradually allowed to reach room temperature, and stirred for 2 h. The reaction mixture was cooled to 0° C. and quenched with saturated aqueous Na₂SO₄ solution. The precipitated solids were filtered under vacuum, washed with 10% MeOH in DCM (100 mL), and organic layer was washed with brine (20 mL) and concentrated under reduced pressure to obtain the crude product. The product was purified by silica gel chromatography using EtOAc: hexanes (2:8). The fractions containing the product were combined and concentrated under vacuum to obtain 2-(3-methoxypropoxy) terephthalaldehyde

### Synthesis of 3,3′-((2,2′-(2-(3-methoxypropoxy)-1,4-phenylene)bis(1H-benzo[d]imidazole-6,2-diyl))bis(oxy))bis(N,N-dimethylpropan-1-amine)

In a microwave vial, a mixture of 2-(3-methoxypropoxy) terephthalaldehyde (0.32 g, 1.44 mmol), 4-(3-(dimethylamino) propoxy) benzene-1, 2-diamine (0.66 g, 3.18 mmol) and Na₂S₂O₅ (0.57 g, 2.89 mmol) in anhydrous DMA (8.0 mL) was heated at 170° C. for 35 min. The reaction mixture was cooled to room temperature and the contents were slowly poured into water (50 mL) and stirred for 10 min, whereupon, a brown solid precipitated. The brown solid was filtered under vacuum and washed with water (2×50 mL), and dried to obtain the crude product. The product was purified by mass triggered HPLC. The fractions containing only the pure product were combined for concentration to obtain 3,3′-((2,2′-(2-(3-methoxypropoxy)-1,4-phenylene)bis(1H-benzo[d]imidazole-6,2-diyl))bis(oxy))bis(N,N-dimethylpropan-1-amine).

### Synthesis of 3-(2,5-bis(6-(3-(dimethylamino)propoxy)-1H-benzo[d]imidazol-2-yl)phenoxy)propan-1-ol

To a solution of 3,3′-((2,2′-(2-(3-methoxypropoxy)-1,4-phenylene)bis(1H-benzo[d]imidazole-6,2-diyl))bis(oxy))bis(N,N-dimethylpropan-1-amine (260 mg, 0.43 mmol) in anhydrous CH₂Cl₂ (25.0 mL) at 0° C. under argon atmosphere, was added BBr₃ (2.0 M in CH₂Cl₂, 2.20 mL, 2.15 mmol) in a dropwise manner over a period of 10 min. After complete addition, the mixture was stirred at room temperature for 1 h, neutralized with methanol (5.0 mL), and concentrated under reduced pressure to obtain the crude product. The product was purified by mass triggered HPLC. Fractions containing only the pure product were combined for concentration to obtain 3-(2, 5-bis (6-(3-(dimethylamino) propoxy)-1H-benzo[d]imidazol-2-yl) phenoxy) propan-1-ol (E-1485)

### Synthesis of 3-(2,5-bis(6-(3-(dimethylamino)propoxy)-1H-benzo[d]imidazol-2-yl)phenoxy)propyl Methane Sulfonate

To a stirred solution of 2-(2,5-bis(6-(3-(dimethylamino)propoxy)-1H-benzo[d]imidazol-2-yl)phenoxy)ethanol (150 mg, 0.25 mmol) in anhydrous CH₂Cl₂ (25.0 mL), at 0° C. under N₂ atmosphere, was added Et₃N (0.17 mL, 1.25 mmol) followed with methane sulfonyl chloride (50.0 mg, 0.51 mmol) in a dropwise manner. After complete addition, the reaction mixture was stirred at room temperature for 2 h. The reaction mixture was concentrated under vacuum to obtain 3-(2, 5-bis (6-(3-(dimethylamino) propoxy)-1H-benzo[d]imidazol-2-yl) phenoxy)propyl methane sulfonate The crude product was used directly in the next step.

### Synthesis of 3,3′-((2,2′-(2-(3-azidopropoxy)-1,4-phenylene)bis(1H-benzo[d]imidazole-6,2-diyl))bis(oxy))bis(N,N-dimethylpropan-1-amine))

To a stirred solution of 3-(2,5-bis(6-(3-(dimethylamino)propoxy)-1H-benzo[d]imidazol-2-yl)phenoxy) propyl methane sulfonate (160 mg, 0.24 mmol) in anhydrous DMF (6.0 mL), at room temperature under N₂ atmosphere, was added sodium azide (78 mg, 0.84 mmol), and the reaction mixture was stirred at 60° C. for 12 h. The reaction mixture was cooled to room temperature and the contents were slowly poured into ice-cold water and stirred for 10 min, whereupon a solid precipitated. The solid was collected by filtration under vacuum, washed with water (10 mL) and dried to obtain the crude product. The product was purified by mass triggered HPLC. The fractions containing only the pure product were combined for concentration to obtain the title compound. MS (ESI): m/z=612 [M+H]⁺.

### Example 7 (Compound A1): Synthesis of 3,3′,3″,3′″-(((((oxybis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

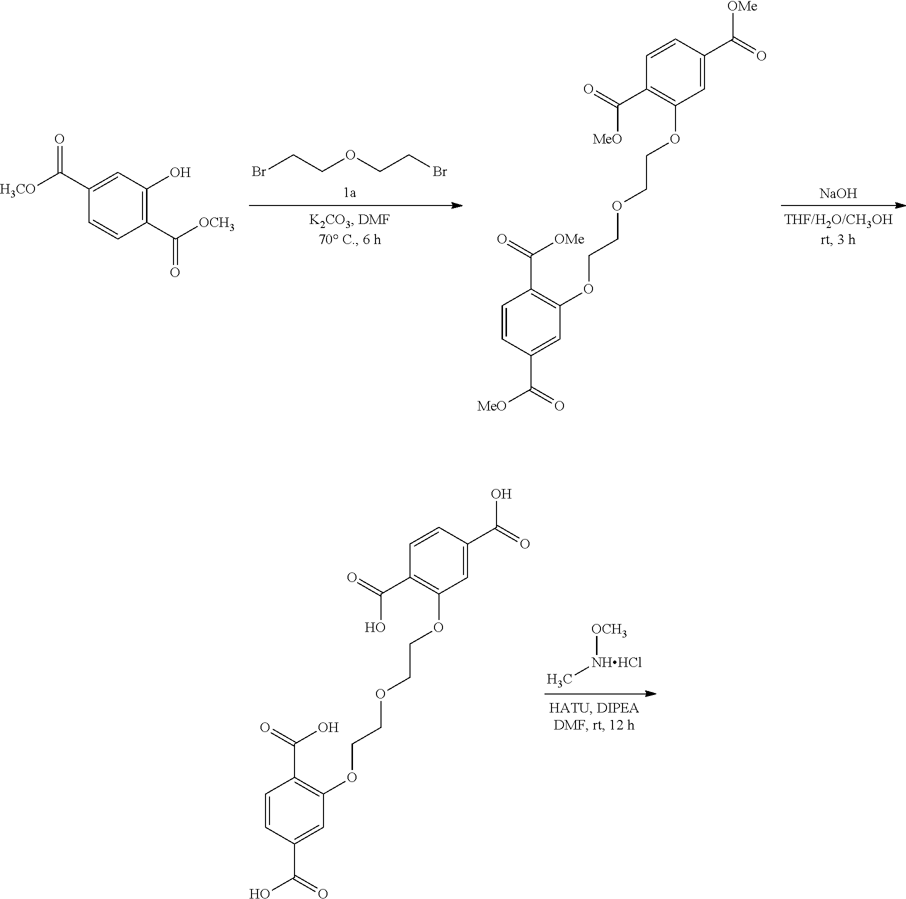

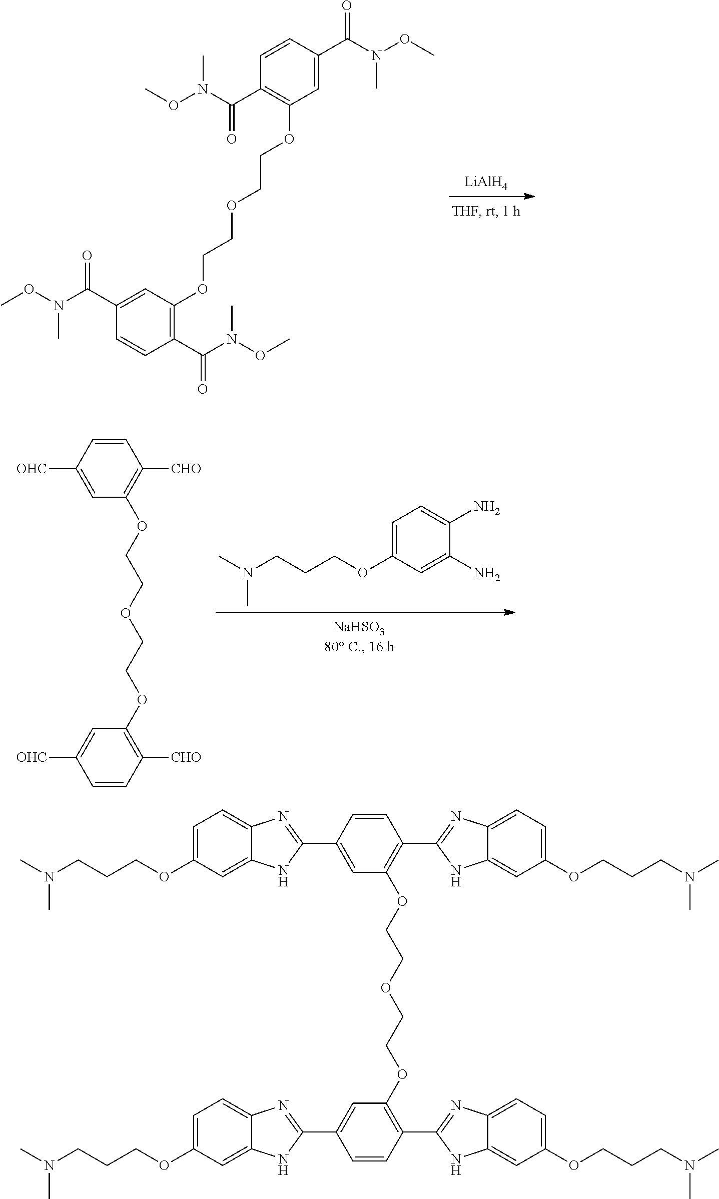

### Synthesis of tetramethyl 2,2′-((oxybis(ethane-2,1-diyl))bis(oxy))diterephthalate

To a stirred solution of dimethyl 2-hydroxyterephthalate (1.00 g, 4.76 mmol) in anhydrous DMF (20.0 mL), at room temperature under N₂ atmosphere, was added K₂CO₃ (1.32 g, 9.52 mmol) followed with 1-bromo-2-(2-bromoethoxy)ethane (0.55 g, 2.37 mmol) and the reaction mixture was stirred at 70° C. for 6 h. The reaction mixture was cooled to room temperature and the contents were poured onto ice cold water (50 mL), and the aqueous phase was extracted with EtOAc (2×50 mL). The combined EtOAc extracts were washed with water (2×50 mL), brine (50 mL), dried over anhydrous Na₂SO₄, filtered and concentrated under vacuum to obtain the crude product. The product was purified by triturating using hot EtOAc (30 mL) to obtain tetramethyl 2,2′-((oxybis(ethane-2,1-diyl))bis(oxy))diterephthalate.

### Synthesis of 2,2′-((oxybis(ethane-2,1-diyl))bis(oxy))diterephthalic Acid

To a stirred solution of tetramethyl 2,2′-((oxybis(ethane-2,1-diyl))bis(oxy))diterephthalate (1.20 g, 2.44 mmol) in a mixture of THF (15.0 mL): CH₃OH (10.0 mL), and H₂O (5.00 mL) was added NaOH (0.49 g, 12.2 mmol) at rt and the mixture was stirred for 3 h. The reaction mixture was concentrated under reduced pressure to remove THF and CH₃OH, and the remaining contents were acidified to pH 2 using 2N aqueous HCl, whereupon, a white solid precipitated. The solid was filtered under vacuum, washed with water (10 mL), and dried to obtain 2,2′-((oxybis(ethane-2,1-diyl))bis(oxy))diterephthalic acid.

### Synthesis of 2,2′-((oxybis(ethane-2,1-diyl))bis(oxy))bis(N¹,N⁴-dimethoxy-N¹,N⁴-dimethylterephthalamide)

To a stirred solution of 2,2′-((oxybis(ethane-2,1-diyl))bis(oxy))diterephthalic acid (0.71 g, 1.63 mmol) in anhydrous DMF (10.0 mL), at 0° C. under N₂ atmosphere, was added DIPEA (1.14 mL, 6.54 mmol), propylphosphonic anhydride solution (2.02 mL, 13.1 mmol) and N, O-dimethylhydroxylamine hydrochloride (0.87 g, 8.99 mmol), and the reaction mixture was stirred at room temperature for 12 h. The product was purified by silica gel chromatography using CH₃OH: CH₂Cl₂ (2:8) to obtain 2,2′-((oxybis(ethane-2,1-diyl))bis(oxy))bis(N¹,N⁴-dimethoxy-N¹,N⁴-dimethylterephthalamide).

### Synthesis of 2,2′-((oxybis(ethane-2,1-diyl))bis(oxy))diterephthalaldehyde

To a stirred solution of 2,2′-((oxybis(ethane-2,1-diyl))bis(oxy))bis(N¹,N⁴-dimethoxy-N¹,N⁴-dimethylterephthalamide) (140 mg, 0.23 mmol) in THF (10.0 mL), at −78° C. under argon atmosphere, was added LiAIH₄ (2.0 M in THF, 1.00 mL, 1.84 mmol) in a dropwise manner over a period of 10 min. The reaction mixture was gradually allowed to reach room temperature, and stirred for 1 h. The reaction mixture was cooled to 0° C. and quenched with saturated aqueous Na₂SO₄ solution, diluted with water (10 mL), and the aqueous phase was extracted with EtOAc (2×20 mL). The combined EtOAc extracts were washed with water (30 mL), dried over anhydrous Na₂SO₄, filtered and concentrated under vacuum to obtain the crude product. The product was purified by silica gel chromatography using EtOAc: Hexanes (1:1). The fractions containing the product were combined and concentrated under vacuum to obtain 2,2′-((oxybis(ethane-2,1-diyl))bis(oxy))diterephthalaldehyde.

### Synthesis of 3,3′,3″,3′″-((((((ethane-1,2-diylbis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 2,2′-(((ethane-1,2-diylbis(oxy))bis(ethane-2,1-diyl))bis(oxy))diterephthalaldehyde (50.0 mg, 1.21 mmol) in anhydrous CH₃OH (10.0 mL) at room temperature under N₂ atmosphere, was added NaHSO₃ (149 mg, 0.78 mmol) followed with 4-(3-(dimethylamino)propoxy)benzene-1,2-diamine (126 mg, 6.03 mmol) and the reaction mixture was stirred at 80° C. for 16 h. The reaction mixture was concentrated under reduced pressure to obtain crude product. The product was purified by mass triggered HPLC. The fractions containing only the pure product were combined for concentration to obtain the title compound. MS (ESI): m/z=1128 [M+H]+.

### Example 8 (Compound A2): Synthesis of 3,3′,3″,3′″-((((((ethane-1,2-diylbis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

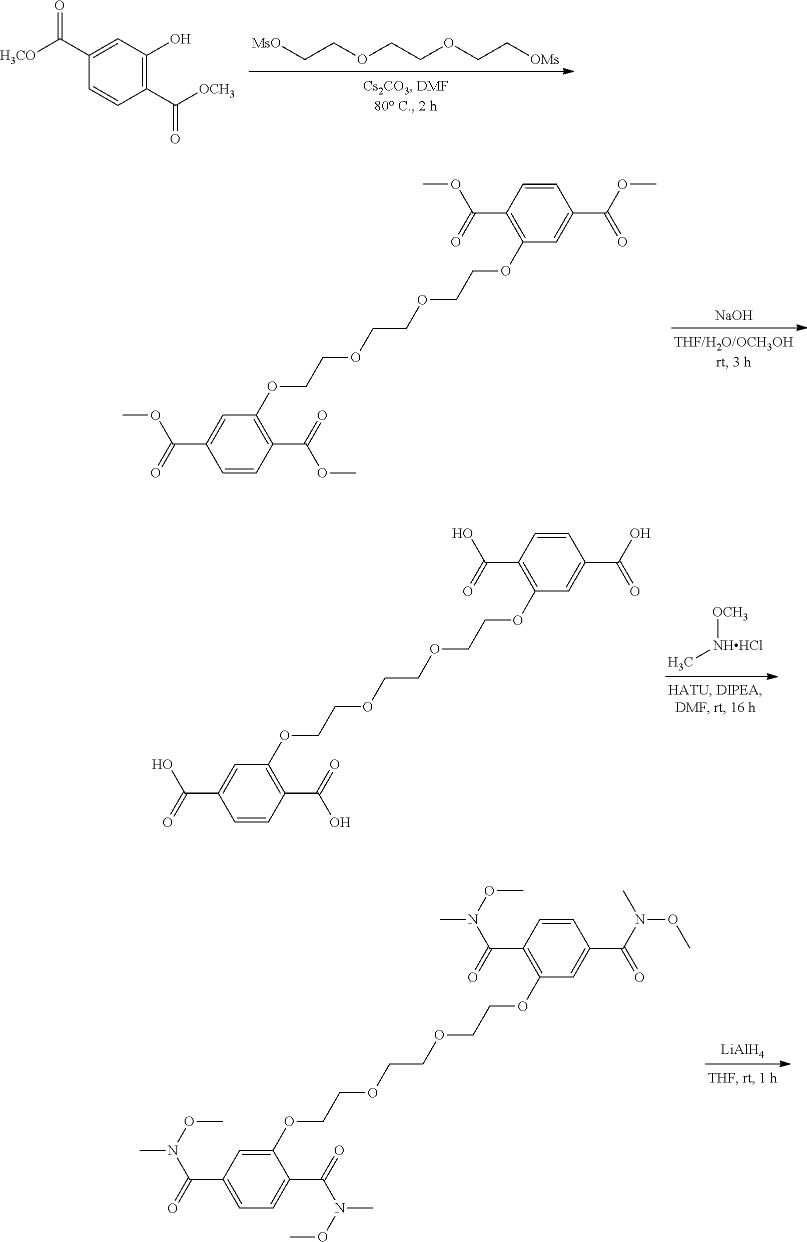

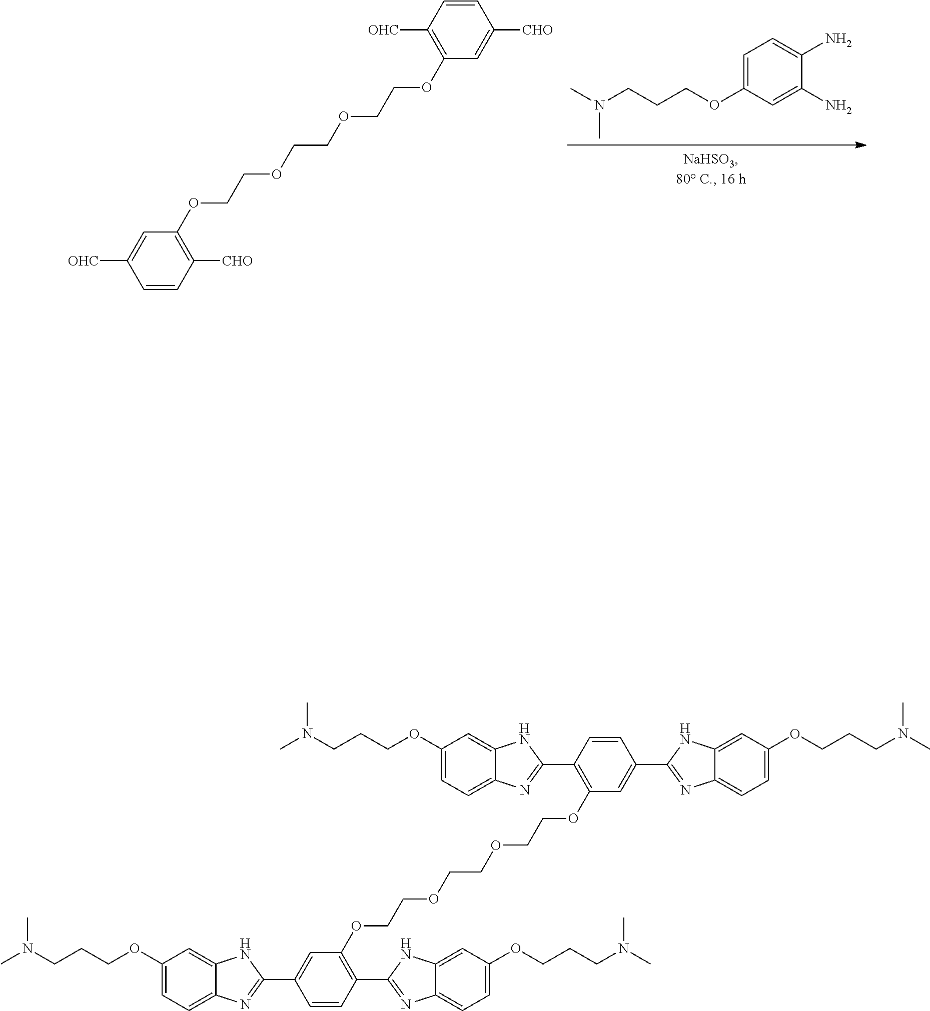

### Synthesis of tetramethyl 2,2′-(((ethane-1,2-diylbis(oxy))bis(ethane-2,1-diyl))bis(oxy))diterephthalate

To a stirred solution of dimethyl 2-hydroxyterephthalate (3.00 g, 14.3 mmol) in anhydfrous DMF (60.0 mL), at room temperature under N₂ atmosphere, was added Cs₂CO₃ (9.30 g, 28.5 mmol) followed with (ethane-1,2-diylbis(oxy))bis(ethane-2, 1-diyl) dimethanesulfonate (3.06 g, 9.99 mmol) and the reaction mixture was stirred at 80° C. for 2 h. The reaction mixture was cooled to room temperature and the contents were poured on to ice cold water (50 mL), and the aqueous phase was extracted with EtOAc (2×50 mL). The combined EtOAc extracts were washed with water (2×50 mL), brine (50 mL), dried over anhydrous Na₂SO₄, filtered and concentrated under vacuum to obtain the crude product. The product was purified by silica gel chromatography using EtOAc: Hexanes (4:6). The fractions containing the product were combined and concentrated under vacuum to obtain tetramethyl 2,2′-(((ethane-1,2-diylbis(oxy))bis(ethane-2,1-diyl))bis(oxy))diterephthalate.

### Synthesis of 2,2′-(((ethane-1,2-diylbis(oxy))bis(ethane-2,1-diyl))bis(oxy))diterephthalic Acid

To a stirred solution of tetramethyl 2,2′-(((ethane-1,2-diylbis(oxy))bis(ethane-2,1-diyl))bis(oxy))diterephthalate (2.00 g, 3.74 mmol) in a mixture of THF (20.0 mL), CH₃OH (20.0 mL), and H₂O (10.0 mL) was added NaOH (0.59 mg, 15.0 mmol), at rt and the mixture was stirred for 3 h. The reaction mixture was concentrated under reduced pressure to remove THF and CH₃OH, and the remaining contents were acidified to pH 2 using 2N aqueous HCl, whereupon, a white solid precipitated. The solid was filtered under vacuum, washed with water (10 mL), and dried to obtain 2,2′-(((ethane-1,2-diylbis(oxy))bis(ethane-2,1-diyl))bis(oxy))diterephthalic acid.

### Synthesis of 2,2′-(((ethane-1,2-diylbis(oxy))bis(ethane-2,1-diyl))bis(oxy))bis(N¹,N-dimethoxy-N¹,N⁴-dimethylterephthalamide)

To a stirred solution of 2,2′-(((ethane-1,2-diylbis(oxy))bis(ethane-2,1-diyl))bis(oxy))diterephthalic acid (2.00 g, 4.18 mmol) in anhydrous DMF (30.0 mL), at 0° C. under N₂ atmosphere, was added HATU (9.54 g, 25.1 mmol), DIPEA (7.30 mL, 41.8 mmol) and N, O-dimethylhydroxylamine hydrochloride (4.08 g, 41.8 mmol), and the reaction mixture was stirred at room temperature for 16 h. The product was purified by silica gel chromatography using CH₃OH: CH₂Cl₂ (1:9) to obtain 2,2′-(((ethane-1,2-diylbis(oxy))bis(ethane-2,1-diyl))bis(oxy))bis(N¹,N⁴-dimethoxy-N¹,N⁴-dimethylterephthalamide). The crude product was directly used in the next step.

### Synthesis of 2,2′-(((ethane-1,2-diylbis(oxy))bis(ethane-2,1-diyl))bis(oxy))diterephthalaldehyde

To a stirred solution of 2,2′-(((ethane-1,2-diylbis(oxy))bis(ethane-2,1-diyl))bis(oxy))bis(N¹,N⁴-dimethoxy-N¹,N⁴-dimethylterephthalamide (400 mg, 6.15 mmol) in THF (20.0 mL), at −78° C. under argon atmosphere, was added LiAIH₄ (2.0 M in THF, 12.0 mL, 24.5 mmol) in a dropwise manner over a period of 10 min. The reaction mixture was gradually allowed to reach room temperature, and stirred for 1 h. The reaction mixture was cooled to 0° C. and quenched with saturated aqueous Na₂SO₄ solution, diluted with water (20 mL), and the aqueous phase was extracted with EtOAc (2×50 mL). The combined EtOAc extracts were washed with water (50 mL), dried over anhydrous Na₂SO₄, filtered and concentrated under vacuum to obtain the crude product. The product was purified by silica gel chromatography using EtOAc: Hexanes (1:1). The fractions containing the product were combined and concentrated under vacuum to obtain 2,2′-(((ethane-1,2-diylbis(oxy))bis(ethane-2,1-diyl))bis(oxy))diterephthalaldehyde.

### Synthesis of 3,3′,3″,3′″-((((((ethane-1,2-diylbis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 2,2′-(((ethane-1,2-diylbis(oxy))bis(ethane-2,1-diyl))bis(oxy))diterephthalaldehyde (50 mg, 1.21 mmol) in CH₃OH (10.0 mL) at room temperature under N₂ atmosphere, was added NaHSO₃ (149 mg, 0.78 mmol) followed with 4-(3-(dimethylamino)propoxy)benzene-1,2-diamine (126 mg, 6.03 mmol) and the reaction mixture was stirred at 80° C. for 16 h. The reaction mixture was concentrated under reduced pressure to obtain crude product. The product was purified by mass triggered HPLC. The fractions containing only the pure product were combined for concentration to obtain the title compound. MS (ESI): m/z=1171 [M+H]⁺.

### Example 9 (Compound A3): Synthesis of 3,3′,3″,3′″-(((((((oxybis(ethane-2,1-diyl))bis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

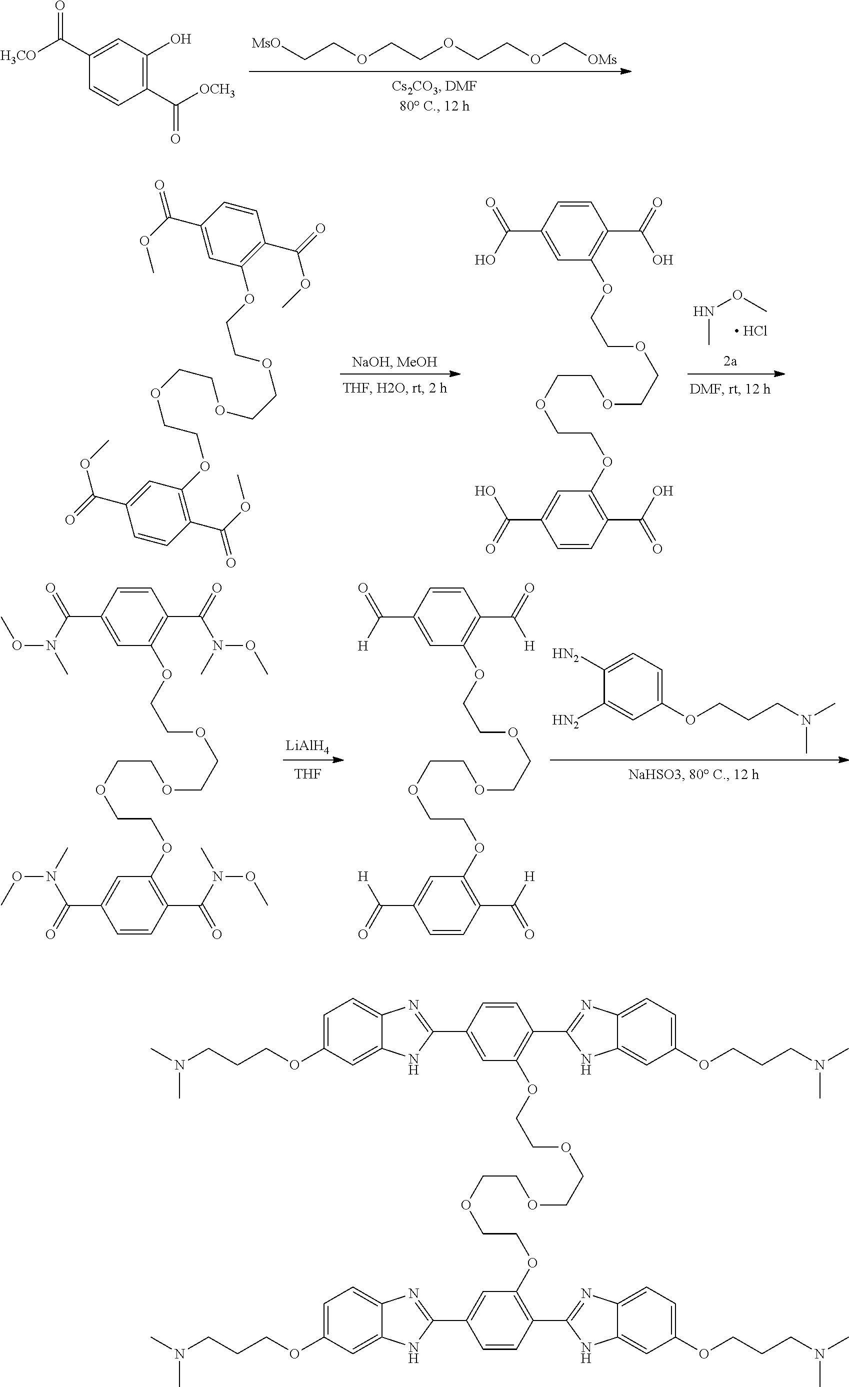

### Synthesis of tetramethyl 2,2′-((((oxybis(ethane-2,1-diyl))bis(oxy))bis(ethane-2, 1-diyl))bis(oxy))diterephthalate

To a stirred solution of dimethyl 2-hydroxyterephthalate (3.00 g, 14.3 mmol) in anhydfrous DMF (30.0 mL), at room temperature under N₂ atmosphere, was added Cs₂CO₃ (9.28 g, 28.5 mmol) followed with ((oxybis(ethane-2,1-diyl))bis(oxy))bis(ethane-2,1-diyl) dimethanesulfonate (2.50 g, 17.1 mmol) and the reaction mixture was stirred at 80° C. for 12 h. The reaction mixture was cooled to room temperature and the contents were poured on to ice cold water (60 mL), and the aqueous phase was extracted with EtOAc (2×60 mL). The combined EtOAc extracts were washed with water (2×50 mL), brine (50 mL), dried over anhydrous Na₂SO₄, filtered and concentrated under vacuum to obtain the crude product. The product was purified by silica gel chromatography using EtOAc: Hexanes (4:6). The fractions containing the product were combined and concentrated under vacuum to tetramethyl 2,2′-((((oxybis(ethane-2,1-diyl))bis(oxy))bis(ethane-2,1-diyl))bis(oxy))diterephthalate

### Synthesis of 2,2′-((((oxybis(ethane-2,1-diyl))bis(oxy))bis(ethane-2,1-diyl))bis(oxy))diterephthalic Acid

To a stirred solution of tetramethyl 2,2′-((((oxybis(ethane-2,1-diyl))bis(oxy))bis(ethane-2,1-diyl))bis(oxy))diterephthalate (2.00 g, 3.46 mmol) in a mixture of THF (30.0 mL), CH₃OH (10.0 mL), and H₂O (10.0 mL) was added NaOH (0.59 mg, 13.9 mmol), at rt and the mixture was stirred for 3 h. The reaction mixture was concentrated under reduced pressure to remove THF and CH₃OH, and the remaining contents were acidified to pH 2 using 2N aqueous HCl, whereupon, a white solid precipitated. The solid was filtered under vacuum, washed with water (10 mL), and dried to obtain 2,2′-((((oxybis(ethane-2,1-diyl))bis(oxy))bis(ethane-2,1-diyl))bis(oxy))diterephthalic acid.

### Synthesis of 2,2′-((((oxybis(ethane-2,1-diyl))bis(oxy))bis(ethane-2,1-diyl))bis(oxy))bis(N1,N4-dimethoxy-N1,N4-dimethylterephthalamide)

To a stirred solution of 2,2′-((((oxybis(ethane-2,1-diyl))bis(oxy))bis(ethane-2,1-diyl))bis(oxy))diterephthalic acid (1.60 g, 3.06 mmol) in anhydrous DMF (20.0 mL), at 0° C. under N₂ atmosphere, was added DIPEA (5.30 mL, 30.6 mmol), propylphosphonic anhydride (8.10 mL, 24.5 mmol), followed with N, O-dimethylhydroxylamine hydrochloride (2.42 g, 24.5 mmol), and the reaction mixture was stirred at room temperature for 12 h. The reaction mixture was concentrated under vacuum and the crude product was purified by silica gel chromatography using CH₃OH: CH₂Cl₂ (2:8) to 2,2′-((((oxybis(ethane-2,1-diyl))bis(oxy))bis(ethane-2,1-diyl))bis(oxy))bis(N1,N4-dimethoxy-N1,N4-dimethylterephthalamide.

### Synthesis of 2,2′-((((oxybis(ethane-2,1-diyl))bis(oxy))bis(ethane-2,1-diyl))bis(oxy))diterephthalaldehyde

To a stirred solution of 2,2′-((((oxybis(ethane-2,1-diyl))bis(oxy))bis(ethane-2,1-diyl))bis(oxy))bis(N1,N4-dimethoxy-N1,N4-dimethylterephthalamide) (1.40, 2.01 mmol) in anhydrous THF (40.0 mL), at −78° C. under argon atmosphere, was added LiAIH₄ (2.0 M in THF, 12.6 mL, 25.2 mmol) in a dropwise manner over a period of 10 min. The reaction mixture was gradually allowed to reach room temperature, and stirred for additional 1h. The reaction mixture was cooled to 0° C. and quenched with saturated aqueous Na₂SO₄ solution, diluted with water (20 mL), and the aqueous phase was extracted with EtOAc (2×100 mL). The combined EtOAc extracts were washed with water (50 mL), dried over anhydrous Na₂SO₄, filtered and concentrated under vacuum to obtain the crude product. The product was purified by silica gel chromatography using EtOAc: Hexanes (4:6). The fractions containing the product were combined and concentrated under vacuum to obtain 2,2′-((((oxybis(ethane-2,1-diyl))bis(oxy))bis(ethane-2,1-diyl))bis(oxy))diterephthalaldehyde.

### Synthesis of 3,3′,3″,3′″-(((((((oxybis(ethane-2,1-diyl))bis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 2,2′-((((oxybis(ethane-2,1-diyl))bis(oxy))bis(ethane-2,1-diyl))bis(oxy))diterephthalaldehyde (110 mg, 0.24 mmol) in anhydrous CH₃OH (15.0 mL) at room temperature under N₂ atmosphere, was added NaHSO₃ (100 mg, 0.96 mmol) followed with 4-(3-(dimethylamino)propoxy)benzene-1,2-diamine (126 mg, 6.03 mmol) and the reaction mixture was stirred at 80° C. for 16 h. The reaction mixture was concentrated under reduced pressure to obtain crude product. The product was purified by mass triggered HPLC. The fractions containing only the pure product were combined for concentration to obtain the title compound. MS (ESI): m/z=1215 [M+H]⁺.

### Example 10 (Compound A19): Synthesis of 3,3′,3″,3′″-((((((1,2-phenylenebis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

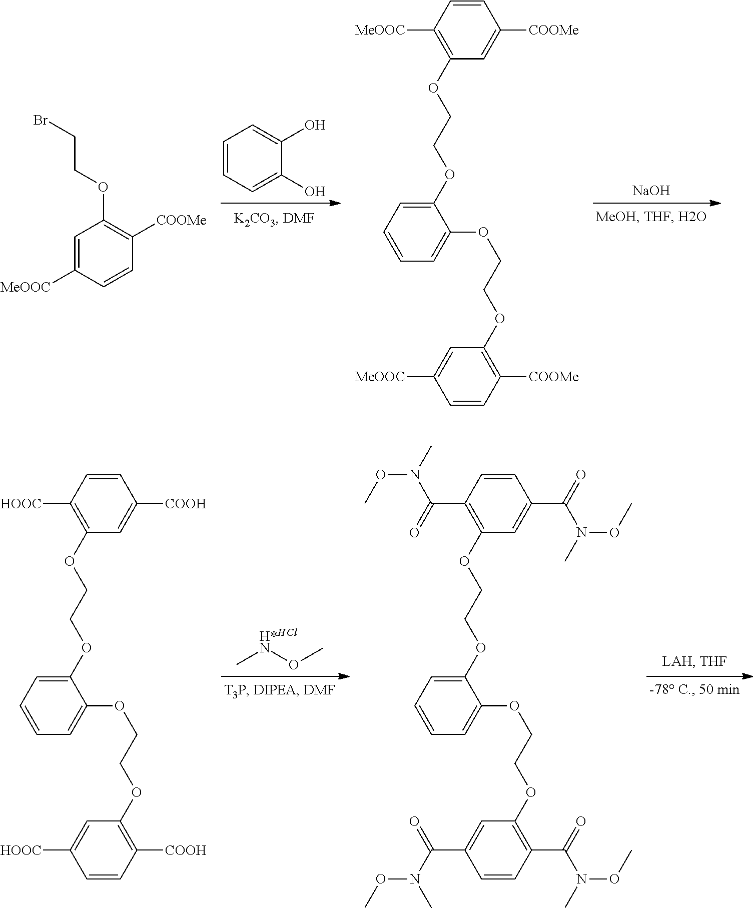

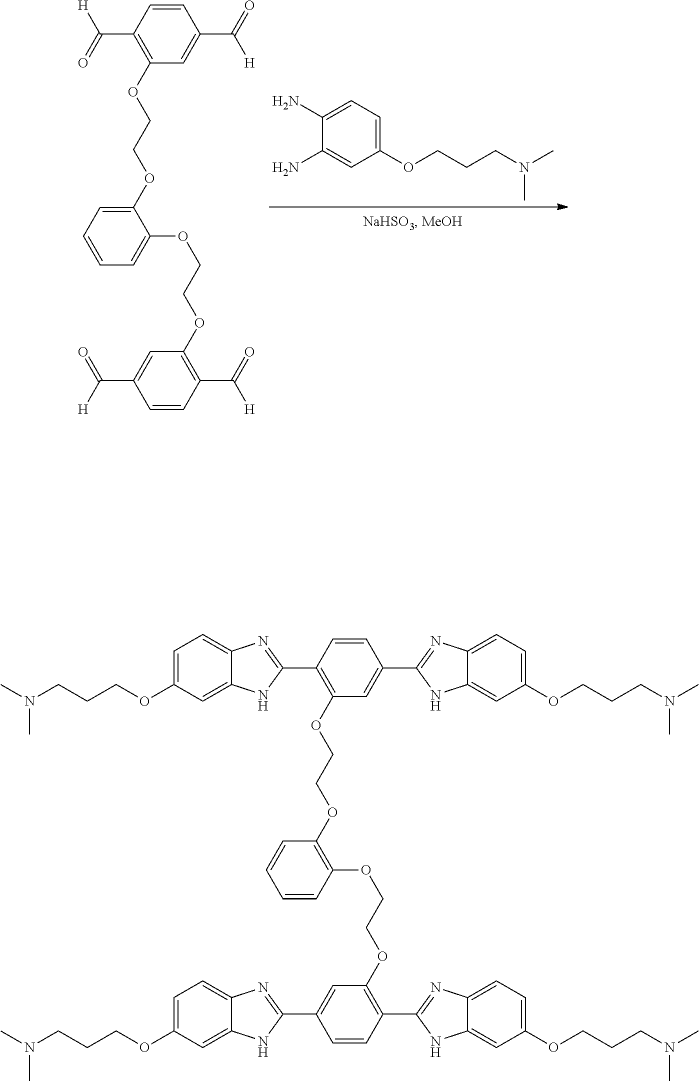

### Synthesis of tetramethyl 2,2′-(((1,2-phenylenebis(oxy))bis(ethane-2,1-diyl))bis(oxy))diterephthalate

To a stirred solution of dimethyl 2-(2-bromoethoxy)terephthalate (2.00 g, 6.30 mmol) in anhydrous DMF (20.0 mL) at room temperature under N₂ atmosphere, was added K₂CO₃ (2.60 g, 18.8 mmol) followed with pyrocatechol (0.35 g, 3.15 mmol) and the reaction mixture was stirred at 70° C. for 6 h. The reaction mixture was filtered through a celite-pad, and the pad was washed with EtOAc (40 mL). The filtrate was concentrated under vacuum to obtain the crude product. The product was purified by silica gel chromatography using EtOAc: Hexane (4:6) Fractions containing the product were combined for concentration to obtain tetramethyl 2,2′-(((1,2-phenylenebis(oxy))bis(ethane-2,1-diyl))bis(oxy))diterephthalate Synthesis of 2,2′-(((1,2-phenylenebis(oxy))bis(ethane-2,1-diyl))bis(oxy))diterephthalic acid:

To a stirred solution of tetramethyl 2,2′-(((1,2-phenylenebis(oxy))bis(ethane-2,1-diyl))bis(oxy))diterephthalate (1.00 g, 1.72 mmol) in a mixture of THF (20.0 mL), H₂O (6.00 mL) and MeOH (5.00 mL) at 0° C. was added NaOH (0.28 g, 6.88 mmol) and the mixture was stirred at rt for 2 h. The reaction mixture was concentrated under reduced pressure to remove volatile solvents, and the aqueous phase was acidified to pH 2 using 2N aqueous HCl, whereupon, a white solid precipitated. The solid was filtered under vacuum, washed with water (10 mL), and dried to obtain 2,2′-(((1,2-phenylenebis(oxy))bis(ethane-2,1-diyl))bis(oxy))diterephthalic acid.

### Synthesis of 2,2′-(((1,2-phenylenebis(oxy))bis(ethane-2,1-diyl))bis(oxy))bis(N1,N4-dimethoxy-N1,N4-dimethylterephthalamide)

To a stirred solution of 2,2′-(((1,2-phenylenebis(oxy))bis(ethane-2,1-diyl))bis(oxy))diterephthalic acid (0.80 g, 1.52 mmol) in anhydrous DMF (15.0 mL), at 0° C. under N₂ atmosphere, was added DIPEA (2.60 mL, 15.2 mmol), T3P (50% in EtOAc, 7.2 mL, 12.2 mmol) and N,O-dimethylhydroxylamine (1.96 g, 12.2 mmol), and the reaction mixture was stirred at room temperature for 12 h. The reaction mixture was concentrated under reduced pressure to obtain the crude product. The product was purified by silica gel chromatography using CH₃OH: CH₂Cl₂ (2:8). The fractions containing the product were combined for concentration to obtain 2,2′-(((1,2-phenylenebis(oxy))bis(ethane-2,1-diyl))bis(oxy))bis(N1,N4-dimethoxy-N1,N4-dimethylterephthalamide)

### Synthesis of 2,2′-(((1,2-phenylenebis(oxy))bis(ethane-2,1-diyl))bis(oxy))diterephthalaldehyde

To a stirred solution of 2,2′-(((1,2-phenylenebis(oxy))bis(ethane-2,1-diyl))bis(oxy))bis(N1,N4-dimethoxy-N1,N4-dimethylterephthalamide) (440 mg, 0.63 mmol) in dry THF (10.0 mL), at −78° C. under argon atmosphere, was added LiAIH₄ (2.0 M in THF, 3.78 mL, 6.87 mmol) in a dropwise manner over a period of 10 min. The reaction mixture was gradually allowed to reach room temperature, and stirred for 3 h. The reaction mixture was cooled to 0° C. and gradually quenched with EtOAc (20 mL), and diluted with water (40 mL). The layers were separated and the aqueous phase was extracted with EtOAc (2×50 mL) and dried over anhydrous Na₂SO₄, filtered and concentrated under vacuum to obtain the crude product. The product was purified by silica gel chromatography using EtOAc: Hexanes (4:6). The fractions containing the product were combined and concentrated under vacuum to obtain 2,2′-(((1,2-phenylenebis(oxy))bis(ethane-2,1-diyl))bis(oxy))diterephthalaldehyde.

### Synthesis of 3,3′,3″,3′″-((((((1,2-phenylenebis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 2,2′-(((1,2-phenylenebis(oxy))bis(ethane-2,1-diyl))bis(oxy))diterephthalaldehyde (35 mg, 0.08 mmol) in anhydrous CH₃OH (10.0 mL) at room temperature under N₂ atmosphere, was added NaHSO₃ (31.5 mg, 0.30 mmol) followed with 4-(3-(dimethylamino)propoxy)benzene-1,2-diamine (79.5 mg, 0.38 mmol) and the reaction mixture was stirred at 80° C. for 12 h. The reaction mixture was concentrated under reduced pressure to obtain crude product. The product was purified by mass triggered HPLC. The fractions containing only the pure product were combined for concentration to obtain the title compound MS (ESI): m/z=1219 [M+H]+.

### Example 11 (Compound A7): Synthesis of 3,3′,3″,3′″-((((((1,3-phenylenebis(oxy))bis(ethane-2,1-diyl)) bis(oxy))bis(benzene-2,1,4-triyl))tetrakis(1H-benzo[d]imidazole-2,6-diyl))tetrakis(oxy))tetrakis(N,N-dimethylpropan-1-amine)

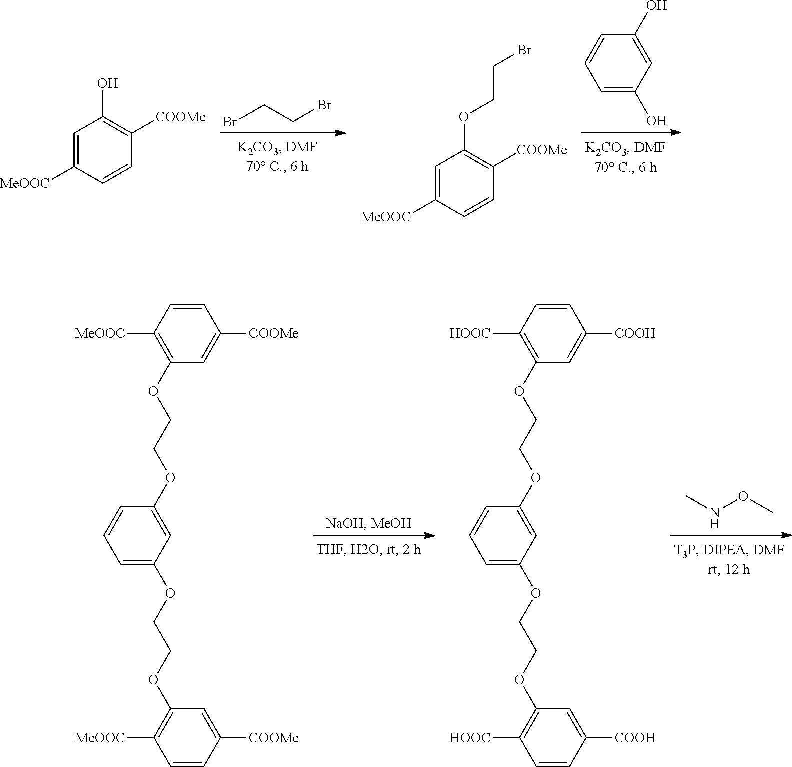

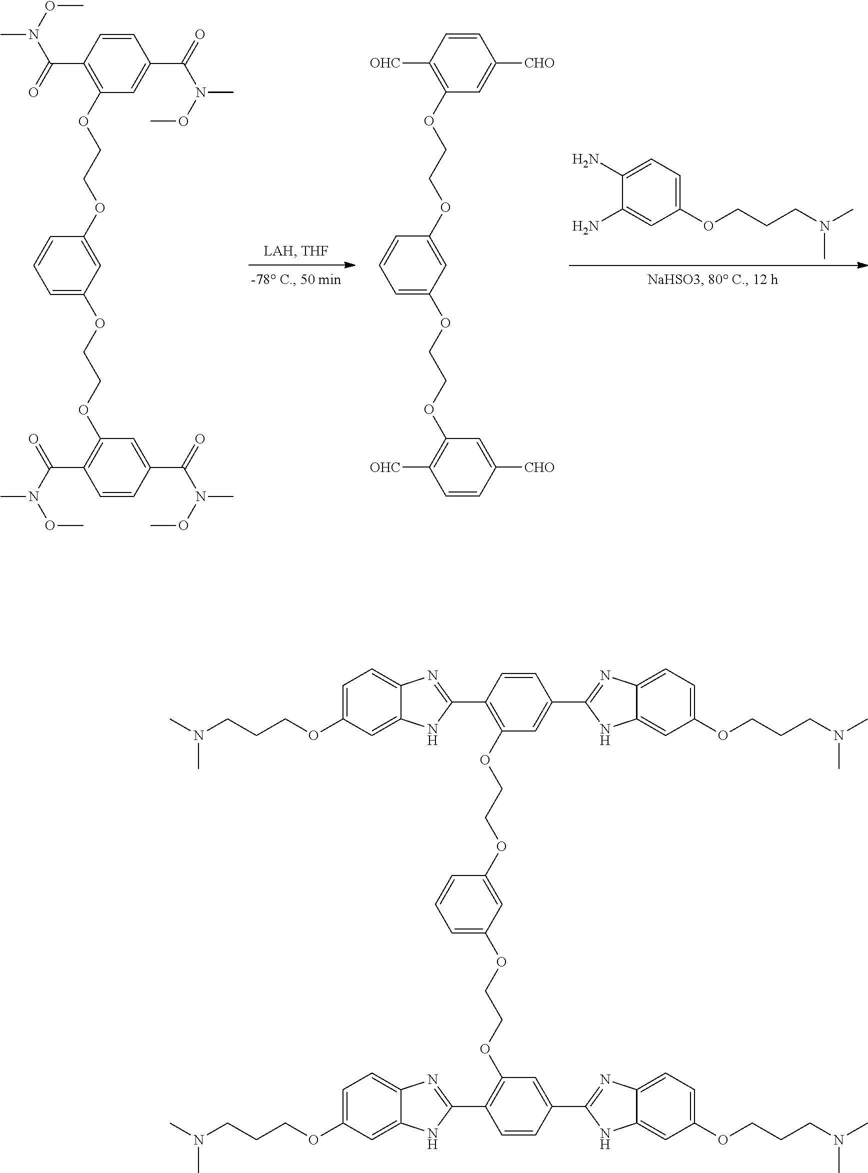

### Synthesis of dimethyl 2-(2-bromoethoxy)terephthalate

To a stirred solution of dimethyl 2-hydroxyterephthalate (1.60 g, 7.61 mmol) in anhydrous DMF (5.0 mL) at room temperature under N₂ atmosphere, was added K₂CO₃ (2.10 g, 15.2 mmol) followed with 1,2-dibromoethane (4.29 g, 22.84 mmol) and the reaction mixture was stirred at 70° C. for 6 h. The reaction mixture was filtered through a celite-pad, and the pad was washed with EtOAc (40 mL). The filtrate was concentrated under vacuum to obtain the crude product. The product was purified by silica gel chromatography using EtOAc: Hexane (2:8). Fractions containing the product were combined for concentration to obtain dimethyl 2-(2-bromoethoxy)terephthalate

### Synthesis of tetramethyl 2,2′-(((1,3-phenylenebis(oxy))bis(ethane-2,1-diyl))bis(oxy))diterephthalate

To a stirred solution of resorcinol (0.17 g, 1.57 mmol) in anhydrous DMF (10.0 mL) at room temperature under N₂ atmosphere, was added K₂CO₃ (1.74 g, 12.6 mmol) followed with dimethyl 2-(2-bromoethoxy)terephthalate (1.00 g, 3.15 mmol) and the reaction mixture was stirred at 70° C. for 6 h. The reaction mixture was cooled to room temperature and filtered through a celite-pad, and the pad was washed with EtOAc (50 mL). The filtrate was concentrated under vacuum to obtain the crude product. The product was purified by silica gel chromatography using EtOAc: Hexane (4:6). Fractions containing the product were combined for concentration to obtain tetramethyl 2,2′-(((1,3-phenylenebis(oxy))bis(ethane-2,1-diyl))bis(oxy))diterephthalate tetramethyl 2,2′-(((1,3-phenylenebis(oxy))bis(ethane-2,1-diyl))bis(oxy))diterephthalate.

### Synthesis of 2,2′-(((1,3-phenylenebis(oxy))bis(ethane-2,1-diyl))bis(oxy))diterephthalic Acid

To a stirred solution of tetramethyl 2,2′-(((1,3-phenylenebis(oxy))bis(ethane-2,1-diyl))bis(oxy))diterephthalate (0.80 g, 1.373 mmol) in a mixture of THF (10.0 mL), H₂O (6.00 mL) and MeOH (3.00 mL) at 0° C. was added NaOH (0.94 g, 23.5 mmol) and the mixture was stirred at rt for 2 h. The reaction mixture was concentrated under reduced pressure to remove volatile solvents, and the aqueous phase was acidified to pH 2 using 2N aqueous HCl, whereupon, a white solid precipitated. The solid was filtered under vacuum, washed with water (10 mL), and dried to obtain 2,2′-(((1,3-phenylenebis(oxy))bis(ethane-2,1-diyl))bis(oxy))diterephthalic acid.

### Synthesis of 2,2′-(((1,3-phenylenebis(oxy))bis(ethane-2,1-diyl))bis(oxy))bis(N1,N4-dimethoxy-N1,N4-dimethylterephthalamide)

To a stirred solution of 2,2′-(((1,3-phenylenebis(oxy))bis(ethane-2,1-diyl))bis(oxy))diterephthalic acid (0.64 g, 1.21 mmol) in anhydrous DMF (15.0 mL), at 0° C. under N₂ atmosphere, was added DIPEA (1.69 mL, 9.73 mmol), T3P (50% in EtOAc, 1.50 mL, 9.73 mmol) and N,O-dimethylhydroxylamine (0.59 g, 9.73 mmol), and the reaction mixture was stirred at room temperature for 12 h. The reaction mixture was concentrated under reduced pressure to obtain the crude product. The product was purified by silica gel chromatography using CH₃OH: CH₂Cl₂ (1:9). The fractions containing the product were combined for concentration to obtain 2,2′-(((1,3-phenylenebis(oxy))bis(ethane-2,1-diyl))bis(oxy))bis(N1,N4-dimethoxy-N1,N4-dimethylterephthalamide.

### Synthesis of 2,2′-(((1,3-phenylenebis(oxy))bis(ethane-2,1-diyl))bis(oxy))diterephthalaldehyde

To a stirred solution of 2,2′-(((1,3-phenylenebis(oxy))bis(ethane-2,1-diyl))bis(oxy))bis(N1,N4-dimethoxy-N1,N4-dimethylterephthalamide) (400 mg, 0.57 mmol) in dry THF (10.0 mL), at −78° C. under argon atmosphere, was added LiAIH₄ (2.0 M in THF, 3.43 mL, 6.87 mmol) in a dropwise manner over a period of 10 min. The reaction mixture was gradually allowed to reach room temperature, and stirred for 3 h. The reaction mixture was cooled to 0° C. and gradually quenched with EtOAc (20 mL), and diluted with water (40 mL). The layers were separated and the aqueous phase was extracted with EtOAc (2×50 mL). The combined EtOAc extracts were washed with water (50 mL), dried over anhydrous Na₂SO₄, filtered and concentrated under vacuum to obtain the crude product. The product was purified by silica gel chromatography using EtOAc: Hexanes (1:2). The fractions containing the product were combined and concentrated under vacuum to obtain 2,2′-(((1,3-phenylenebis(oxy))bis(ethane-2,1-diyl))bis(oxy))diterephthalaldehyde

### Synthesis of 3,3′,3″,3′″-((((((1,3-phenylenebis(oxy))bis(ethane-2,1-diyl)) bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 2,2′-(((1,3-phenylenebis(oxy))bis(ethane-2,1-diyl))bis(oxy))diterephthalaldehyde (75 mg, 0.16 mmol) in anhydrous CH₃OH (10.0 mL) at room temperature under N₂ atmosphere, was added NaHSO₃ (67.5 mg, 0.64 mmol) followed with 4-(3-(dimethylamino)propoxy)benzene-1,2-diamine (204 mg, 0.97 mmol) and the reaction mixture was stirred at 80° C. for 12 h. The reaction mixture was concentrated under reduced pressure to obtain crude product. The product was purified by mass triggered HPLC. The fractions containing only the pure product were combined for concentration to obtain the title compound. MS (ESI): m/z=1220 [M+H]+.

### Example 12 (Compound 18): Synthesis of 3,3′,3″,3′″-((((((1,4-phenylenebis(oxy))bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

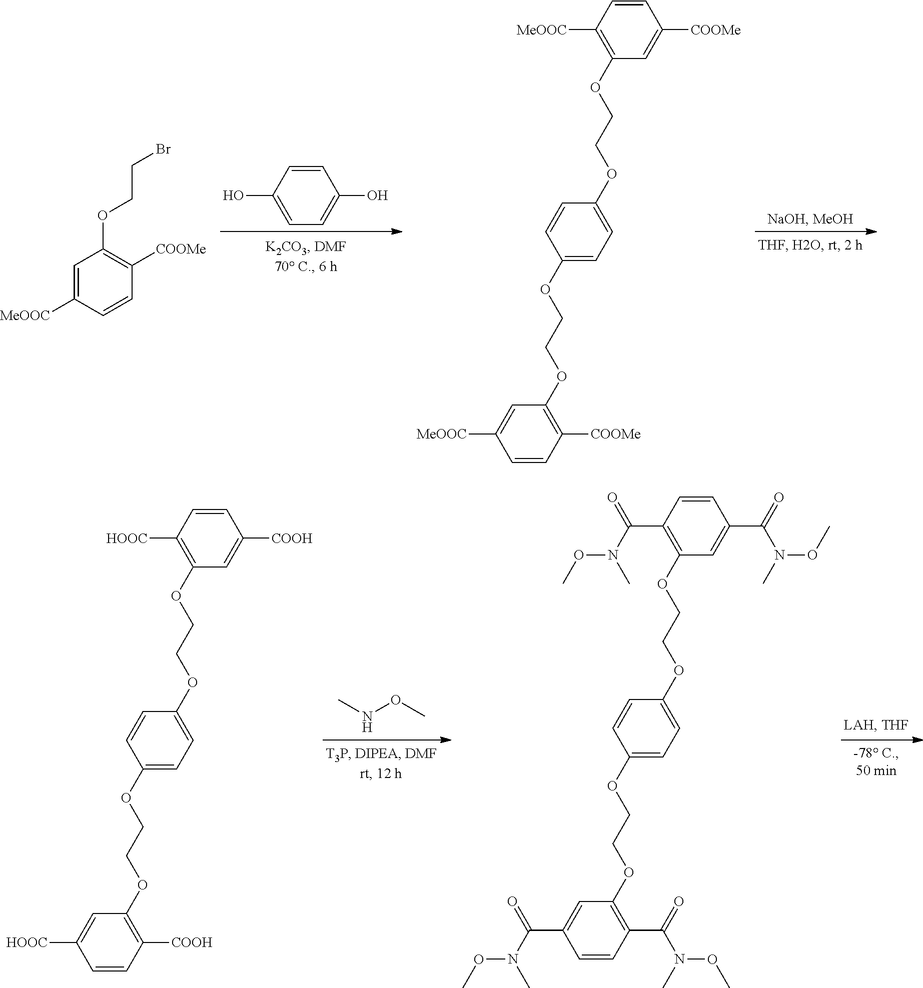

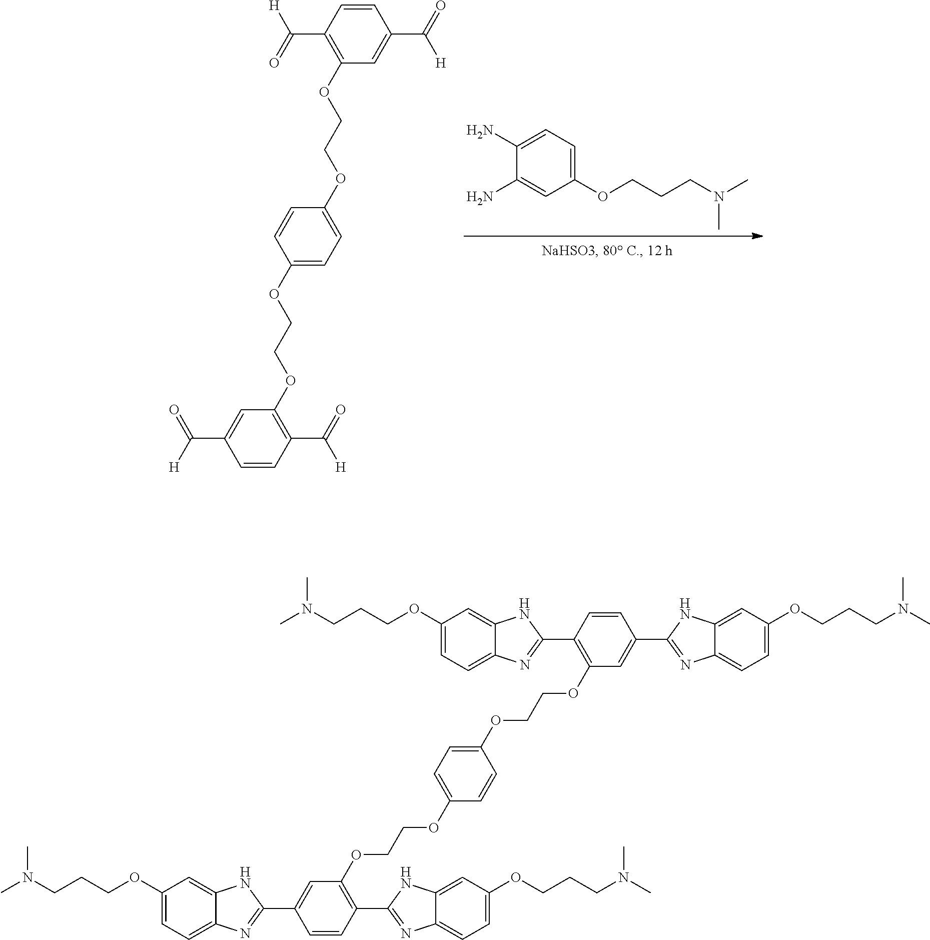

### Synthesis of tetramethyl 2,2′-(((1,4-phenylenebis(oxy))bis(ethane-2, 1-diyl))bis(oxy))diterephthalate

To a stirred solution of hydroquinone (0.36 g, 3.31 mmol) in anhydrous DMF (25.0 mL) at room temperature under N₂ atmosphere, was added K₂CO₃ (2.75 g, 19.87 mmol) followed with dimethyl 2-(2-bromoethoxy)terephthalate (2.10 g, 6.62 mmol) and the reaction mixture was stirred at 80° C. for 16 h. The reaction mixture was cooled to room temperature and filtered through a celite-pad, and the pad was washed with EtOAc (50 mL). The filtrate was concentrated under vacuum to obtain the crude product. The product was purified by silica gel chromatography using EtOAc: Hexane (4:6). Fractions containing the product were combined for concentration to obtain 2,2′-(((1,4-phenylenebis(oxy))bis(ethane-2,1-diyl))bis(oxy))diterephthalate.

### Synthesis of 2,2′-(((1,4-phenylenebis(oxy))bis(ethane-2,1-diyl))bis(oxy))diterephthalic Acid

To a stirred solution of tetramethyl 2,2′-(((1,4-phenylenebis(oxy))bis(ethane-2,1-diyl))bis(oxy))diterephthalate (0.72 g, 1.23 mmol) in a mixture of THF (10.0 mL), H₂O (6.00 mL) and MeOH (3.00 mL) at 0° C. was added NaOH (0.05 g, 1.23 mmol) and the mixture was stirred at rt for 3 h. The reaction mixture was concentrated under reduced pressure to remove volatile solvents, and the aqueous phase was acidified to pH 2 using 2N aqueous HCl, whereupon, a white solid precipitated. The solid was filtered under vacuum, washed with water (10 mL), and dried to obtain 2,2′-(((1,4-phenylenebis(oxy))bis(ethane-2,1-diyl))bis(oxy))diterephthalic acid.

### Synthesis of 2,2′-(((1,4-phenylenebis(oxy))bis(ethane-2,1-diyl))bis(oxy))bis(N1,N4-dimethoxy-N1,N4-dimethylterephthalamide)

To a stirred solution of 2,2′-(((1,4-phenylenebis(oxy))bis(ethane-2,1-diyl))bis(oxy))diterephthalic acid (0.57 g, 1.08 mmol) in anhydrous DMF (15.0 mL), at 0° C. under N₂ atmosphere, was added DIPEA (1.89 mL, 10.73 mmol), T₃P (50% in EtOAc, 6.60 mL, 8.66 mmol) followed with N,O-dimethylhydroxylamine (0.85 g, 8.66 mmol), and the reaction mixture was stirred at room temperature for 12 h. The reaction mixture was concentrated under reduced pressure to obtain the crude product. The product was purified by silica gel chromatography using CH₃OH: CH₂Cl₂ (1:9). The fractions containing the product were combined for concentration to obtain 2,2′-(((1,4-phenylenebis(oxy))bis(ethane-2,1-diyl))bis(oxy))bis(N1,N4-dimethoxy-N1,N4-dimethylterephthalamide).

### Synthesis of 2,2′-(((1,4-phenylenebis(oxy))bis(ethane-2,1-diyl))bis(oxy))diterephthalaldehyde

To a stirred solution of 2,2′-(((1,4-phenylenebis(oxy))bis(ethane-2,1-diyl))bis(oxy))bis(N1,N4-dimethoxy-N1,N4-dimethylterephthalamide) (200 mg, 0.28 mmol) in dry THF (5.0 mL), at −78° C. under argon atmosphere, was added LiAIH₄ (2.0 M in THF, 0.86 mL, 1.71 mmol) in a dropwise manner over a period of 10 min. The reaction mixture was gradually allowed to reach room temperature, and stirred for 3 h. The reaction mixture was cooled to 0° C. and slowly quenched with EtOAc (20 mL), and diluted with water (30 mL). The layers were separated and the aqueous phase was extracted with EtOAc (2×50 mL). The combined EtOAc extracts were washed with water (50 mL), dried over anhydrous Na₂SO₄, filtered and concentrated under vacuum to obtain the crude product. The product was purified by silica gel chromatography using EtOAc: Hexanes (1:2). The fractions containing the product were combined and concentrated under vacuum to obtain 2,2′-(((1,4-phenylenebis(oxy))bis(ethane-2,1-diyl))bis(oxy))diterephthalaldehyde.

### Synthesis of 3,3′,3″,3′″-((((((1,3-phenylenebis(oxy))bis(ethane-2,1-diyl)) bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 2,2′-(((1,4-phenylenebis(oxy))bis(ethane-2,1-diyl))bis(oxy))diterephthalaldehyde (35 mg, 0.07 mmol) in anhydrous CH₃OH (10.0 mL) at room temperature under N₂ atmosphere, was added NaHSO₃ (31.5 mg, 0.30 mmol) followed with 4-(3-(dimethylamino)propoxy)benzene-1,2-diamine (79 mg, 0.37 mmol) and the reaction mixture was stirred at 80° C. for 12 h. The reaction mixture was concentrated under reduced pressure to obtain crude product. The product was purified by mass triggered HPLC. The fractions containing only the pure product were combined for concentration to obtain the title compound. MS (ESI): m/z=1219 [M+H]+.

### Example 13 (Compound A22): Synthesis of 3,3′-((2,2′-(2-(3-(4-((2,5-bis(6-(3-(dimethylamino)propoxy)-1H-benzo[d]imidazol-2-yl)phenoxy)methyl)-1H-1,2,3-triazol-1-yl)propoxy)-1,4-phenylene)bis(1H-benzo[d]imidazole-6,2-diyl))bis(oxy))bis(N,N-dimethylpropan-1-amine)

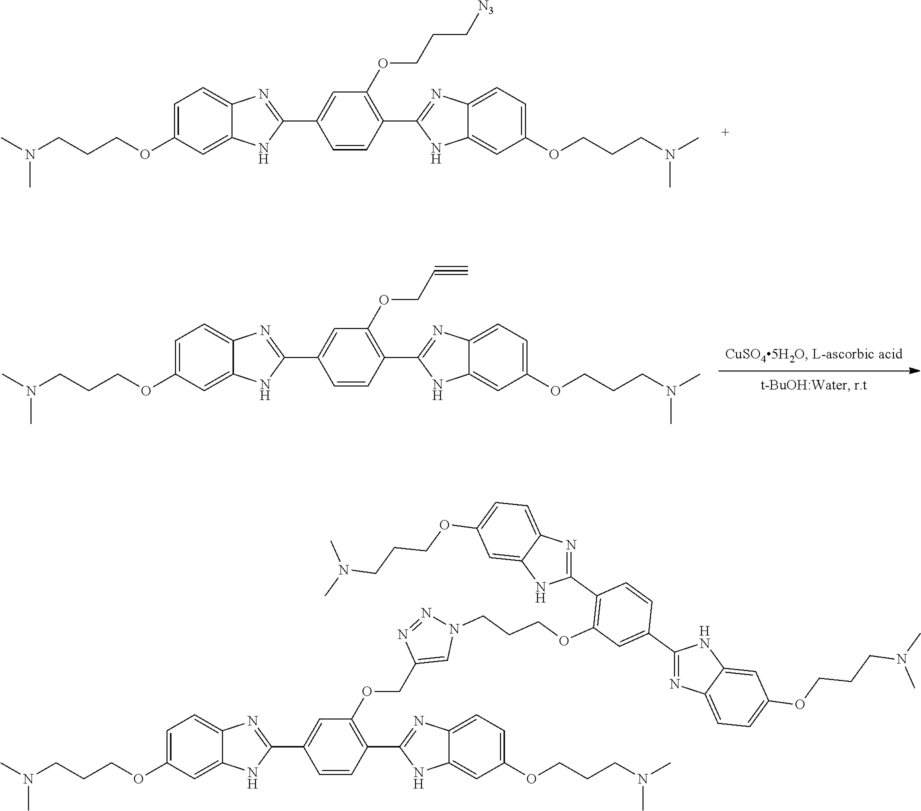

### Synthesis of 3,3′-((2,2′-(2-(3-(4-((2,5-bis(6-(3-(dimethylamino)propoxy)-1H-benzo[d]imidazol-2-yl)phenoxy)methyl)-1H-1,2,3-triazol-1-yl)propoxy)-1,4-phenylene)bis(1H-benzo[d]imidazole-6,2-diyl))bis(oxy))bis(N,N-dimethylpropan-1-amine)

To a stirred solution of 3,3′-((2,2′-(2-(3-azidopropoxy)-1,4-phenylene)bis(1H-benzo[d]imidazole-6,2-diyl))bis(oxy))bis(N,N-dimethylpropan-1-amine) (25.0 mg, 0.04 mmol) and 3,3′-((2,2′-(2-(prop-2-yn-1-yloxy)-1,4-phenylene)bis(1H-benzo[d]imidazole-6,2-diyl))bis(oxy))bis(N,N-dimethylpropan-1-amine)(22.3 mg, 0.04 mmol) in a mixture of tert-BuOH (0.5 ml) and H₂O (0.5 ml) was added copper(II) sulfate pentahydrate (1.52 mg, 0.006 mmol) followed with L-ascorbic acid sodium salt (1.62 mg, 0.008 mmol), and the mixture was stirred at room temperature for 16 h. The reaction mixture was concentrated under vacuum to obtain the crude product. The product was purified by mass triggered prep-HPLC. The fractions containing only the pure product were combined and concentration to obtain the title compound. MS (ESI): m/z=1178 [M+H]⁺.

### Example 14 (Compound A23): Synthesis of 3,3′-((2,2′-(2-((1-(2-(2,5-bis(6-(3-(dimethylamino)propoxy)-1H-benzo[d]imidazol-2-yl)phenoxy)ethyl)-1H-1,2,3-triazol-4-yl)methoxy)-1,4-phenylene)bis(1H-benzo[d]imidazole-6,2-diyl))bis(oxy))bis(N,N-dimethylpropan-1-amine)

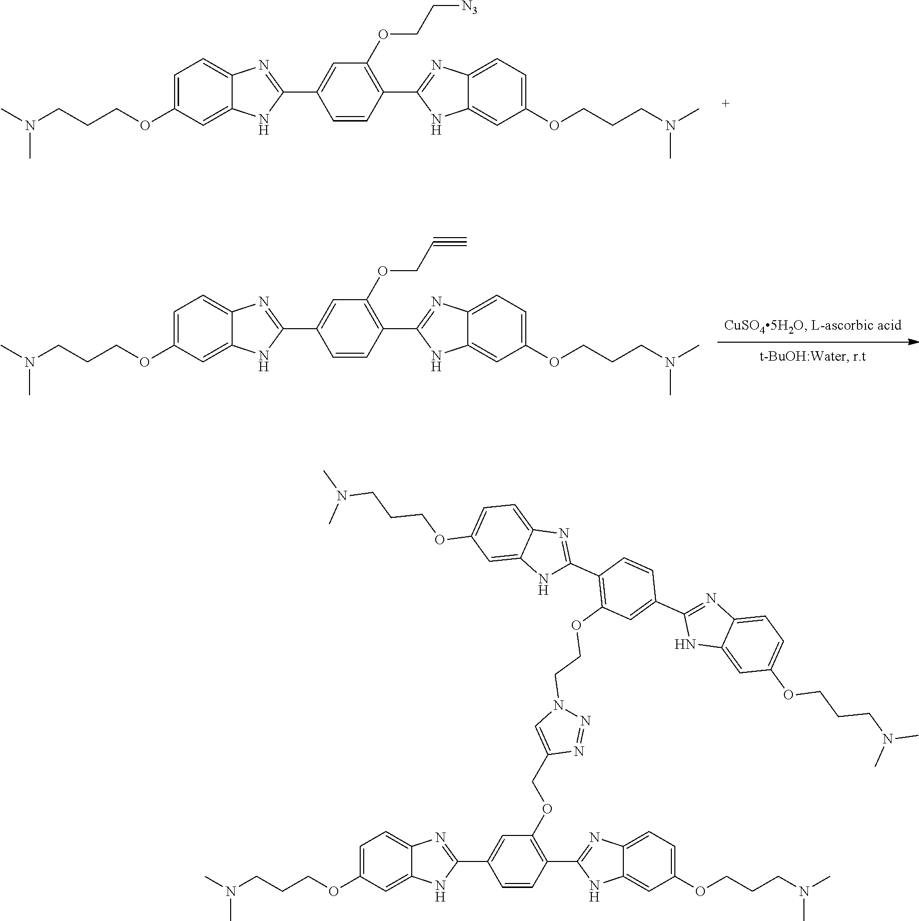

### Synthesis of 3,3′-((2,2′-(2-((1-(2-(2,5-bis(6-(3-(dimethylamino)propoxy)-1H-benzo[d]imidazol-2-yl)phenoxy)ethyl)-1H-1,2,3-triazol-4-yl)methoxy)-1,4-phenylene)bis(1H-benzo[d]imidazole-6,2-diyl))bis(oxy))bis(N,N-dimethylpropan-1-amine)

To a stirred solution of 3,3′-((2,2′-(2-(2-azidoethoxy)-1,4-phenylene)bis(1H-benzo[d]imidazole-6,2-diyl))bis(oxy))bis(N,N-dimethylpropan-1-amine) (25.0 mg, 0.04 mmol) and 3,3′-((2,2′-(2-(prop-2-yn-1-yloxy)-1,4-phenylene)bis(1H-benzo[d]imidazole-6,2-diyl))bis(oxy))bis(N,N-dimethylpropan-1-amine) (23.7 mg, 0.04 mmol) in a mixture of tert-BuOH (0.5 mL) and H₂O (0.5 mL) was added copper(II) sulfate pentahydrate (1.52 mg, 0.006 mmol) followed with L-ascorbic acid sodium salt (1.62 mg, 0.008 mmol), and the mixture was stirred at room temperature for 16 h. The reaction mixture was concentrated under vacuum to obtain the crude product. The product was purified by mass triggered prep-HPLC. The fractions containing only the pure product were combined and concentrated under vacuum to obtain the title compound. MS (ESI): m/z=1164 [M+H]⁺.

### Example 15 (Compound A20): Synthesis of 3,3′-((2,2′-(2-(3-(4-(2-(2,5-bis(6-(3-(dimethylamino)propoxy)-1H-benzo[d]imidazol-2-yl)phenoxy)ethyl)-1H-1,2,3-triazol-1-yl)propoxy)-1,4-phenylene)bis(1H-benzo[d]imidazole-6,2-diyl))bis(oxy))bis(N,N-dimethylpropan-1-amine)

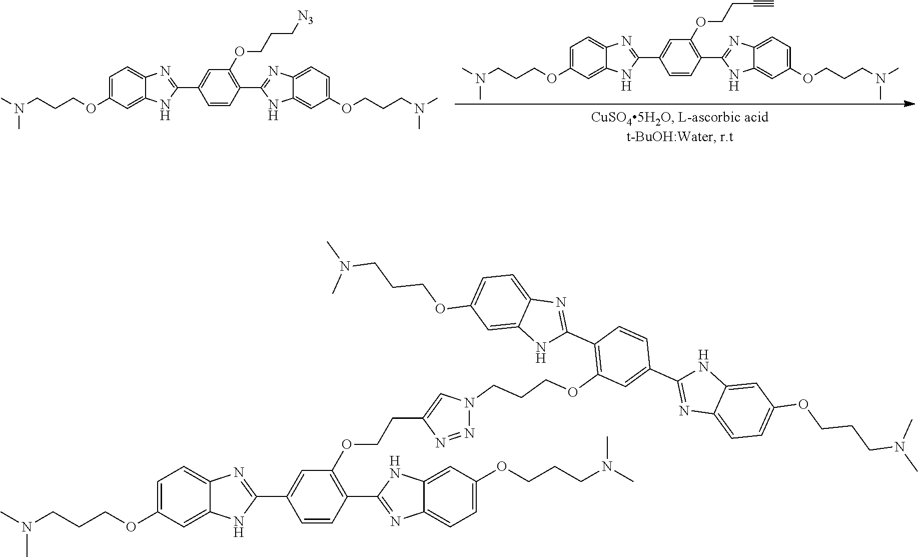

### Synthesis of 3,3′-((2,2′-(2-(3-(4-(2-(2,5-bis(6-(3-(dimethylamino)propoxy)-1H-benzo[d]imidazol-2-yl)phenoxy)ethyl)-1H-1,2,3-triazol-1-yl)propoxy)-1,4-phenylene)bis(1H-benzo[d]imidazole-6,2-diyl))bis(oxy))bis(N,N-dimethylpropan-1-amine)

To a stirred solution of 3,3′-((2,2′-(2-(3-azidopropoxy)-1,4-phenylene)bis(1H-benzo[d]imidazole-6,2-diyl))bis(oxy))bis(N,N-dimethylpropan-1-amine) (15.0 mg, 0.02 mmol) and 3,3′-((2,2′-(2-(but-3-yn-1-yloxy)-1,4-phenylene)bis(1H-benzo[d]imidazole-6,2-diyl))bis(oxy))bis(N,N-dimethylpropan-1-amine) (15.8 mg, 0.02 mmol) in a mixture of tert-BuOH (0.5 mL) and H₂O (0.5 mL) was added copper(II) sulfate pentahydrate (1.22 mg, 0.005 mmol) followed with L-ascorbic acid sodium salt (1.32 mg, 0.006 mmol), and the mixture was stirred at room temperature for 16 h. The reaction mixture was concentrated under vacuum to obtain the crude product. The product was purified by mass triggered prep-HPLC. The fractions containing only the pure product were combined and concentration to obtain the title compound. MS (ESI): m/z=1192 [M+H]⁺.

### Example 16 (Compound A21): Synthesis of 3,3′,3″,3′″-((((((1H-1,2,3-triazole-1,4-diyl)bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

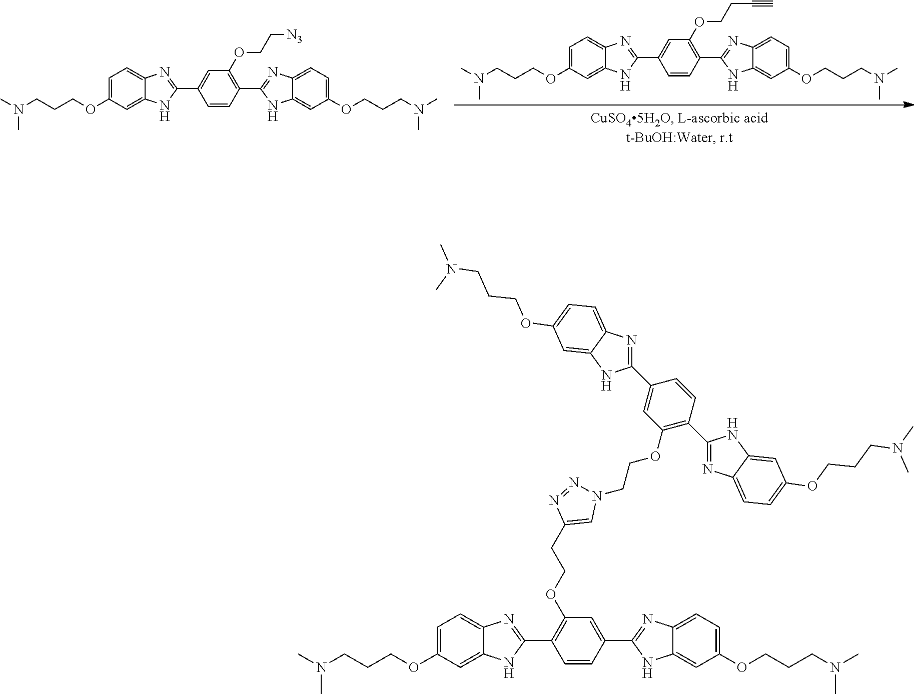

### Synthesis of 3,3′,3″,3′″-((((((1H-1,2,3-triazole-1,4-diyl)bis(ethane-2,1-diyl))bis(oxy))bis(benzene-2,1,4-triyl))tetrakis(1H-benzo[d]imidazole-2,6-diyl))tetrakis(oxy))tetrakis(N,N-dimethylpropan-1-amine)

To a stirred solution of 3,3′-((2,2′-(2-(3-azidopropoxy)-1,4-phenylene)bis(1H-benzo[d]imidazole-6,2-diyl))bis(oxy))bis(N,N-dimethylpropan-1-amine) (20.0 mg, 0.03 mmol) and 3,3′-((2,2′-(2-(but-3-yn-1-yloxy)-1,4-phenylene)bis(1H-benzo[d]imidazole-6,2-diyl))bis(oxy))bis(N,N-dimethylpropan-1-amine) (20.5 mg, 0.03 mmol) in a mixture of tert-BuOH (1.00 mL) and H₂O (1.00 mL) was added copper(II) sulfate pentahydrate (1.20 mg, 0.005 mmol) followed with L-ascorbic acid sodium salt (1.30 mg, 0.006 mmol), and the mixture was stirred at room temperature for 16 h. The reaction mixture was concentrated under vacuum to obtain the crude product. The product was purified by mass triggered prep-HPLC. The fractions containing only the pure product were combined for concentration to obtain the title compound. MS (ESI): m/z=1178 [M+H]⁺.

### Example 17 (Compound A15): Synthesis N,N′-(ethane-1,2-diyl)bis(2-(2,5-bis(6-(3-(dimethylamino)propoxy)-1H-benzo[d]imidazol-2-yl)phenoxy)acetamide)

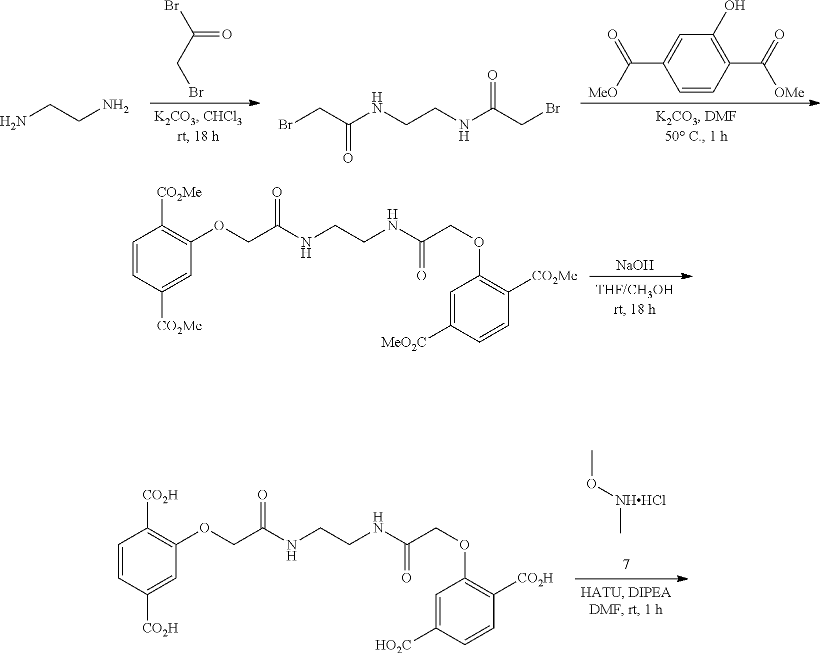

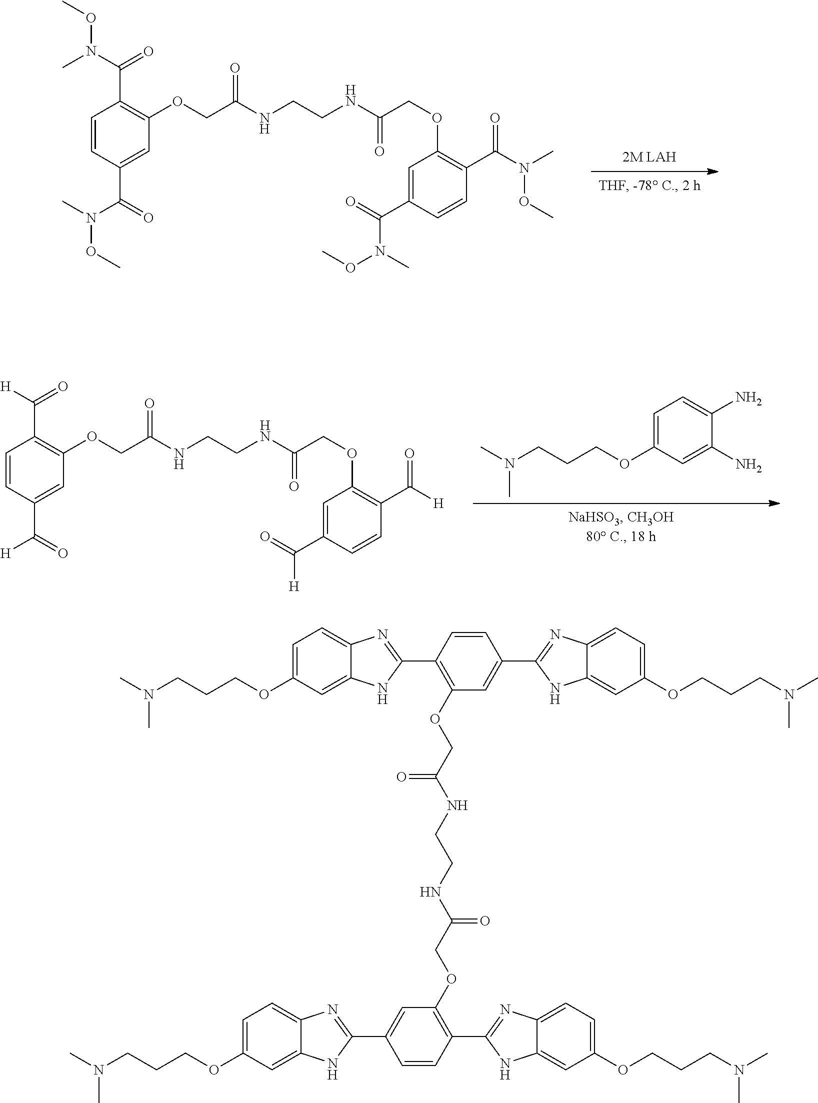

### Synthesis of N,N′-(ethane-1,2-diyl)bis(2-bromoacetamide)

To a stirred solution of ethane-1,2-diamine (3.01 g, 50 mmol) in anhydrous CHCl₃ (100 mL) at room temperature, was added a solution of K₂CO₃ (2.75 g, 19.87 mmol) in water (100 mL). The mixture was cooled to 5° C., and a solution of 2-bromoacetyl bromide (13.1 mL, 150 mmol) in CHCl₃ (100 mL) was added in a drop wise manner over period of 30 min. After complete addition, the cooling bath was removed and the reaction mixture was stirred at room temperature for 18 h. The product precipitated from the reaction mixture, which was filtered under vacuum, washed with water (50 mL) and dried for 24 h to obtain N,N′-(ethane-1,2-diyl)bis(2-bromoacetamide).

### Synthesis of tetramethyl 2,2′-(((ethane-1,2-diylbis(azanediyl))bis(2-oxoethane-2,1-diyl))bis(oxy))diterephthalate

To a stirred solution of dimethyl 2-hydroxyterephthalate (278 mg, 1.33 mmol) in anhydrous DMF (5.0 mL) at room temperature under N₂ atmosphere, was added K₂CO₃ (275 mg, 1.99 mmol) followed with N,N′-(ethane-1,2-diyl)bis(2-bromoacetamide) (200 mg, 0.66 mmol) and the reaction mixture was stirred at 50° C. for 1 h. The reaction mixture was cooled to room temperature and diluted with water (20 mL), whereupon the product precipitated as a white solid. The precipitated solids were filtered under vacuum, washed with EtOAc (10 mL) and dried to obtain tetramethyl 2,2′-(((ethane-1,2-diylbis(azanediyl))bis(2-oxoethane-2,1-diyl))bis(oxy))diterephthalate.

### Synthesis of 2,2′-(((ethane-1,2-diylbis(azanediyl))bis(2-oxoethane-2,1-diyl))bis(oxy))diterephthalic Acid

To a stirred solution of tetramethyl 2,2′-(((ethane-1,2-diylbis(azanediyl))bis(2-oxoethane-2,1-diyl))bis(oxy))diterephthalate (300 mg, 0.54 mmol) in a mixture of THF (10.0 mL), CH₂Cl₂ (3.00 mL) and MeOH (10 mL) at room temperature was added 3M aqueous NaOH (1.00 mL, 3.21 mmol) and the mixture was stirred at room temperature for 8 h. The reaction mixture was concentrated under reduced pressure to remove volatile solvents, and the aqueous phase was acidified to pH 2 using 2N aqueous HCl, whereupon, a white solid precipitated. The solid was filtered under vacuum, washed with water (10 mL), and dried to obtain 2,2′-(((ethane-1,2-diylbis(azanediyl))bis(2-oxoethane-2,1-diyl))bis(oxy))diterephthalic acid.

### Synthesis of 2,2′-(((ethane-1,2-diylbis(azanediyl))bis(2-oxoethane-2,1-diyl))bis(oxy))bis(N1,N4-dimethoxy-N1,N4-dimethylterephthalamide)

To a stirred solution of 2,2′-(((ethane-1,2-diylbis(azanediyl))bis(2-oxoethane-2,1-diyl))bis(oxy))diterephthalic acid (250 mg, 0.50 mmol) in anhydrous DMF (10.0 mL) at rt under N₂ atmosphere, was added DIPEA (0.87 mL, 4.96 mmol), HATU (942 mg, 2.48 mmol) followed with N,O-dimethylhydroxylamine (242 mg, 2.48 mmol), and the reaction mixture was stirred at room temperature for 2 h. The reaction mixture was concentrated under reduced pressure to obtain the crude product. The product was purified by silica gel chromatography using CH₃OH: CH₂Cl₂ (1:9). The fractions containing the product were combined for concentration to obtain 2,2′-(((ethane-1,2-diylbis(azanediyl))bis(2-oxoethane-2,1-diyl))bis(oxy))bis(N1,N4-dimethoxy-N1,N4-dimethylterephthalamide)

### Synthesis of N,N′-(ethane-1,2-diyl)bis(2-(2,5-diformylphenoxy)acetamide)

To a stirred solution of 2,2′-(((ethane-1,2-diylbis(azanediyl))bis(2-oxoethane-2,1-diyl))bis(oxy))bis(N1,N4-dimethoxy-N1,N4-dimethylterephthalamide) (300 mg, 0.44 mmol) in dry THF (10.0 mL), at −78° C. under argon atmosphere, was added LiAIH₄ (2.0 M in THF, 1.78 mL, 3.55 mmol) in a dropwise manner over a period of 10 min, and the reaction mixture was stirred for 2 h. The reaction mixture was slowly quenched with aq. saturated Na₂SO₄ solution and diluted with CH₂Cl₂ (20 mL), and the reaction mixture was filtered through celite bed and filterate was concentrated under vacuum to obtain the crude product. The product was purified by silica gel chromatography using CH₃OH: CH₂Cl₂ (1:9). The fractions containing the product were combined for concentration to obtain N,N′-(ethane-1,2-diyl)bis(2-(2,5-diformylphenoxy)acetamide).

### Synthesis of N,N′-(ethane-1,2-diyl)bis(2-(2,5-bis(6-(3-(dimethylamino)propoxy)-1H-benzo[d]imidazol-2-yl)phenoxy)acetamide)

To a stirred solution of N,N′-(ethane-1,2-diyl)bis(2-(2,5-diformylphenoxy)acetamide) (40 mg, 0.09 mmol) in anhydrous CH₃OH (10.0 mL) at room temperature under N₂ atmosphere, was added NaHSO₃ (47.3 mg, 0.45 mmol) followed with 4-(3-(dimethylamino)propoxy)benzene-1,2-diamine (95 mg, 0.45 mmol) and the reaction mixture was stirred at 80° C. for 18 h. The reaction mixture was concentrated under reduced pressure to obtain crude product. The product was purified by mass triggered HPLC. The fractions containing only the pure product were combined for concentration to obtain the title compound. MS (ESI): m/z=1198 [M+H]+.

### Example 18 (Compound A28): Synthesis of 3,3′-((2,2′-((ethane-1,2-diylbis(oxy))bis(2,1-phenylene))bis(1H-benzo[d]imidazole-6,2-diyl))bis(oxy))bis(N,N-dimethylpropan-1-amine)

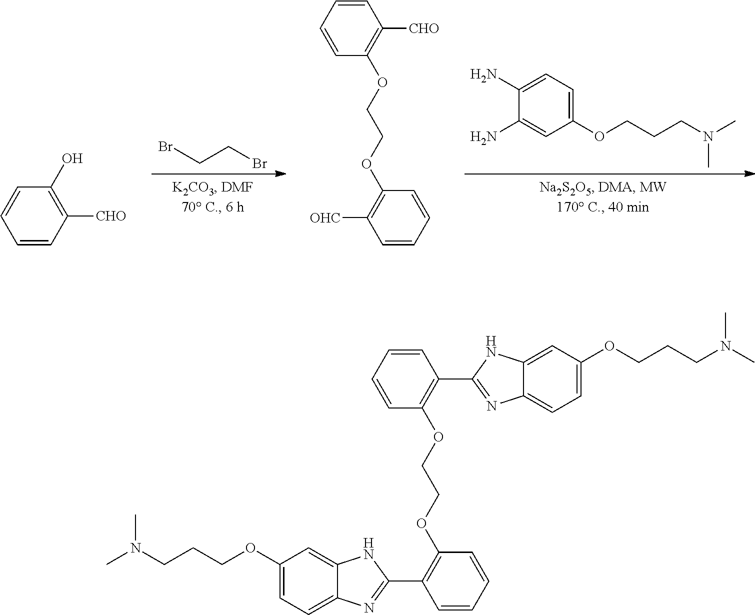

### Synthesis of 2,2′-(ethane-1,2-diylbis(oxy))dibenzaldehyde

To a stirred solution of 2-hydroxybenzaldehyde (500 mg, 4.09 mmol) in DMF (5.00 mL) at room temperature under N₂ atmosphere, was added K₂CO₃ (1.13 g, 8.19 mmol), 1,2-dibromoethane (0.17 mL, 2.04 mmol), and the reaction mixture was heated at 70° C. for 6 h.

The reaction mixture was filtered under vacuum and the solid was washed with hot EtOAc (20 mL). The filtrate was concentrated under vacuum to obtain the crude product which was triturated in hot EtOAc to obtain 2,2′-(ethane-1,2-diylbis(oxy))dibenzaldehyde.

### Synthesis of 3,3′-((2,2′-((ethane-1,2-diylbis(oxy))bis(2,1-phenylene))bis(1H-benzo[d]imidazole-6,2-diyl))bis(oxy))bis(N,N-dimethylpropan-1-amine)

In a microwave vial, a mixture of 2,2′-(ethane-1,2-diylbis(oxy))dibenzaldehyde (200 mg, 0.74 mmol), 4-(3-(dimethylamino)propoxy)benzene-1,2-diamine (325 mg, 1.55 mmol) and Na₂S₂O₅ (281 mg, 1.48 mmol) in anhydrous DMA (5.00 mL) was heated at 170° C. for 40 min. The reaction mixture was cooled to room temperature and the contents were slowly poured into water (50 mL) and stirred for 10 min, whereupon, a brown solid precipitated. The brown solid was filtered under vacuum, washed with water (2×10.0 mL) and dried to obtain the crude product. The product was purified by silicagel chromatography, using CMA (MeOH: CH₂Cl₂: NH₄OH, 1:1.8:0.2) solution. The fractions containing only the pure product were combined for concentration to obtain the title compound. MS (ESI): m/z=649 [M+H]+.

### Example 19 (Compound A8): Synthesis of 3,3′-((2,2′-(((oxybis(ethane-2,1-diyl))bis(oxy))bis(2,1-phenylene))bis(1H-benzo[d]imidazole-6,2-diyl))bis(oxy))bis(N,N-dimethylpropan-1-amine)

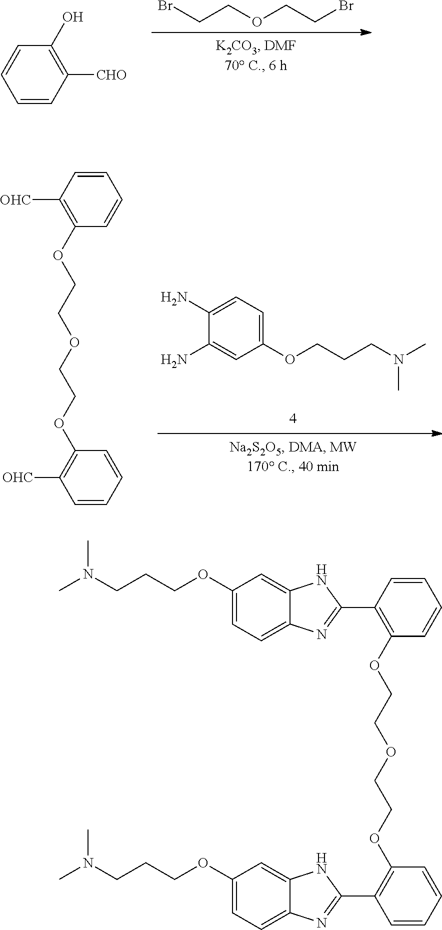

### Synthesis of 2,2′-((oxybis(ethane-2,1-diyl))bis(oxy))dibenzaldehyde

To a stirred solution of 2-hydroxybenzaldehyde (0.50 g, 4.09 mmol) in anhydrous DMF (5.00 mL) at room temperature under N₂ atmosphere, was added K₂CO₃ (1.13 g, 8.19 mmol), 1-bromo-2-(2-bromoethoxy)ethane (0.47 g, 2.04 mmol) and the reaction mixture was heated at 70° C. for 6 h. The reaction mixture was filtered under vacuum and the solid was washed with hot EtOAc (20 mL). The filtrate was concentrated under vacuum to obtain the crude product, which was triturated in hot EtOAc, filtered under vacuum, washed with EtOAc (20 mL), and dried to obtain 2,2′-((oxybis(ethane-2,1-diyl))bis(oxy))dibenzaldehyde.

### Synthesis of 3,3′-((2,2′-(((oxybis(ethane-2,1-diyl))bis(oxy))bis(2,1-phenylene))bis(1H-benzo[d]imidazole-6,2-diyl))bis(oxy))bis(N,N-dimethylpropan-1-amine)

In a microwave vial, a mixture of 2,2′-((oxybis(ethane-2,1-diyl))bis(oxy))dibenzaldehyde (0.20 g, 0.63 mmol), 4-(3-(dimethylamino)propoxy)benzene-1,2-diamine (0.33 g, 1.59 mmol) and Na₂S₂O₅ (0.24 g, 1.27 mmol) in anhydrous DMA (5.00 mL) was heated at 170° C. for 40 min. The reaction mixture was cooled to room temperature and the contents were slowly poured into water (50 mL) and stirred for 10 min, whereupon, a brown solid precipitated. The brown solid was filtered under vacuum, washed with water (2×10 mL) and dried to obtain the crude product. The crude was purified by prep-HPLC (please refer to the perp-HPLC details). The fractions containing only the pure product were combined for concentration to obtain the title compound. MS (ESI): m/z=693 [M+H]+.

### Example 20 (Compounds A9 and A10): Synthesis of 3,3′-((2,2′-((((ethane-1,2-diylbis(oxy))bis(ethane-2,1-diyl))bis(oxy))bis(2,1-phenylene))bis(1H-benzo[d]imidazole-6,2-diyl))bis(oxy))bis(N,N-dimethylpropan-1-amine) (14a) and 2-(2-(2-(2-(2-(2-(6-(3-(dimethylamino)propoxy)-1H-benzo[d]imidazole-2-yl)phenoxy)ethoxy)ethoxy)ethoxy)phenyl)-1H-benzo[d]imidazol-6-ol (14b)

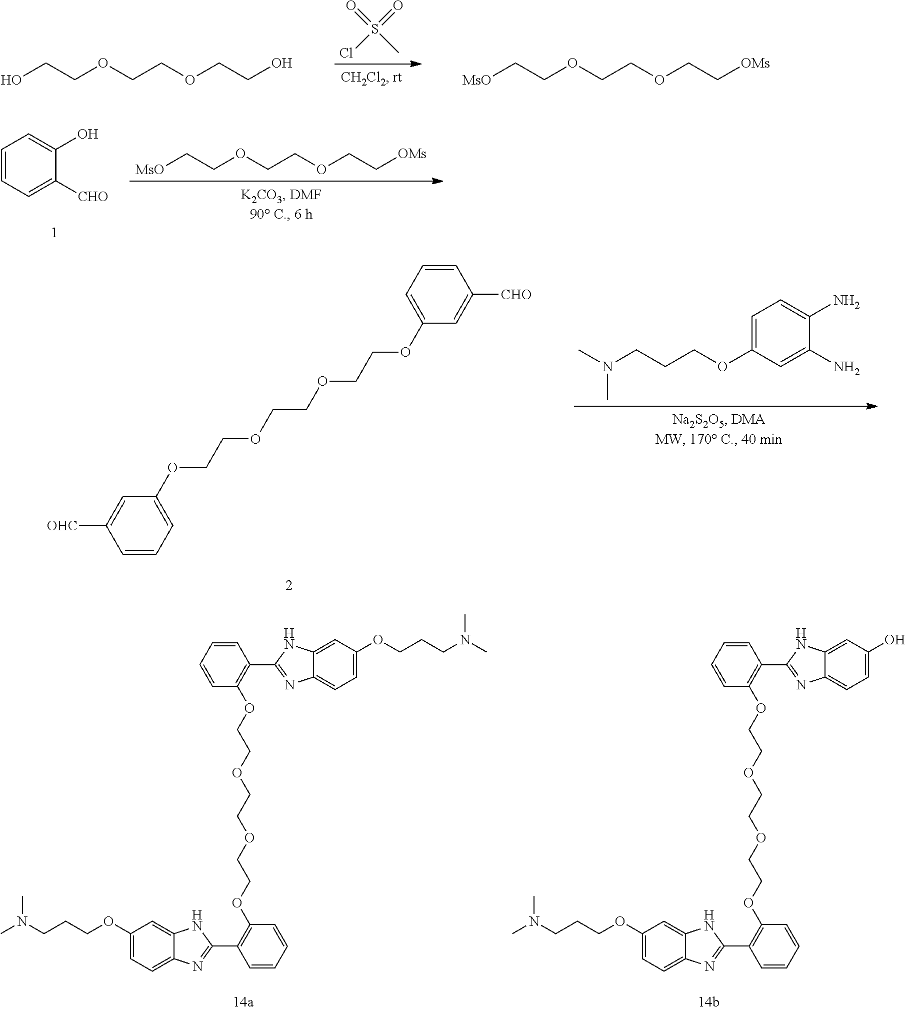

### Synthesis of (ethane-1,2-diylbis(oxy))bis(ethane-2,1-diyl)dimethanesulfonate

To a stirred solution of 2,2′-(ethane-1,2-diylbis(oxy))bis(ethan-1-ol) (5.00 g, 33.3 mmol) in anhydrous CH₂Cl₂ (70.0 mL) at 0° C. under N₂ atmosphere, was added DIPEA (29.1 mL, 166 mmol) followed with methane sulfonyl chloride (12.9 mL, 166 mmol), and the reaction mixture was stirred at room temperature for 4 h. The contents were poured on to ice cold water, and the aqueous phase was extracted with CH₂Cl₂ (2×100 mL). The combined CH₂Cl₂ extracts were washed with saturated aqueous NaHCO₃ solution (50 mL), brine (50 mL), dried over anhydrous Na₂SO₄, filtered and concentrated under vacuum to obtain (ethane-1,2-diylbis(oxy))bis(ethane-2,1-diyl)dimethanesulfonate.

### Synthesis of 3-3′-(((ethane-1,2-diylbis(oxy))bis(ethane-2,1diyl))bis(oxy))dibenzaldehdye

To a stirred solution of 2-hydroxy benzaldehyde (1.00 g, 8.19 mmol) in anhydrous DMF (20.0 mL) at room temperature under N₂ atmosphere, was added K₂CO₃ (3.40 g, 24.5 mmol) followed with (ethane-1,2-diylbis(oxy))bis(ethane-2,1-diyl)dimethanesulfonate (1.75 g, 5.73 mmol) and the reaction mixture was heated at 90° C. for 6 h. The reaction mixture was cooled to room temperature and the contents were poured on to ice cold water, and the aqueous phase was extracted with EtOAc (2×50 mL). The combined EtOAc extracts were washed with brine (50 mL), dried over anhydrous Na₂SO₄, filtered and concentrated under vacuum to obtain the crude product. The product was purified by silica gel chromatography using CH₃OH: CH₂Cl₂ (1:9). The fractions containing the product were combined and concentrated under vacuum to obtain 3-3′-(((ethane-1,2-diylbis(oxy))bis(ethane-2,1-diyl))bis(oxy))dibenzaldehdye.

### Synthesis of 3,3′-((2,2′-((((ethane-1,2-diylbis(oxy))bis(ethane-2,1-diyl))bis(oxy))bis(2,1-phenylene))bis(1H-benzo[d]imidazole-6,2-diyl))bis(oxy))bis(N,N-dimethylpropan-1-amine) (14a) and 2-(2-(2-(2-(2-(2-(6-(3-(dimethylamino)propoxy)-1H-benzo[d]imidazole-2-yl)phenoxy)ethoxy)ethoxy)ethoxy)phenyl)-1H-benzo[d]imidazol-6-ol (14b)

In a microwave vial, a mixture of 3-3′-(((ethane-1,2-diylbis(oxy))bis(ethane-2,1-diyl))bis(oxy))dibenzaldehdye (300 mg, 0.83 mmol), 4-(3-(dimethylamino)propoxy)benzene-1,2-diamine (438 mg, 2.09 mmol) and Na₂S₂O₅ (318 mg, 1.67 mmol) in anhydrous DMA (5.00 mL) was heated at 170° C. for 40 min. The reaction mixture was cooled to r. t and the contents were concentrated under vacuum to obtain the crude product.

The product was purified by mass triggered HPLC. The fractions containing only the pure product were combined for concentration to obtain the title compounds.

3,3′-((2,2′-((((ethane-1,2-diylbis(oxy))bis(ethane-2,1-diyl))bis(oxy))bis(2,1-phenylene))bis(1H-benzo[d]imidazole-6,2-diyl))bis(oxy))bis(N,N-dimethylpropan-1-amine) (A9): MS (ESI): m/z=737 [M+H]⁺.

2-(2-(2-(2-(2-(2-(6-(3-(dimethylamino)propoxy)-1H-benzo[d]imidazole-2-yl)phenoxy)ethoxy)ethoxy)ethoxy)phenyl)-1H-benzo[d]imidazol-6-ol (A10): MS (ESI): m/z=652 [M+H]⁺.

### Example 21 (Compound A11): Synthesis of 3,3′-(((((((oxybis(ethane-2,1-diyl))bis(oxy))bis(ethane-2, 1-diyl))bis(oxy))bis(2,1-phenylene))bis(1H-benzo[d]imidazole-2,6-diyl))bis(oxy))bis(N,N-dimethylpropan-1-amine)

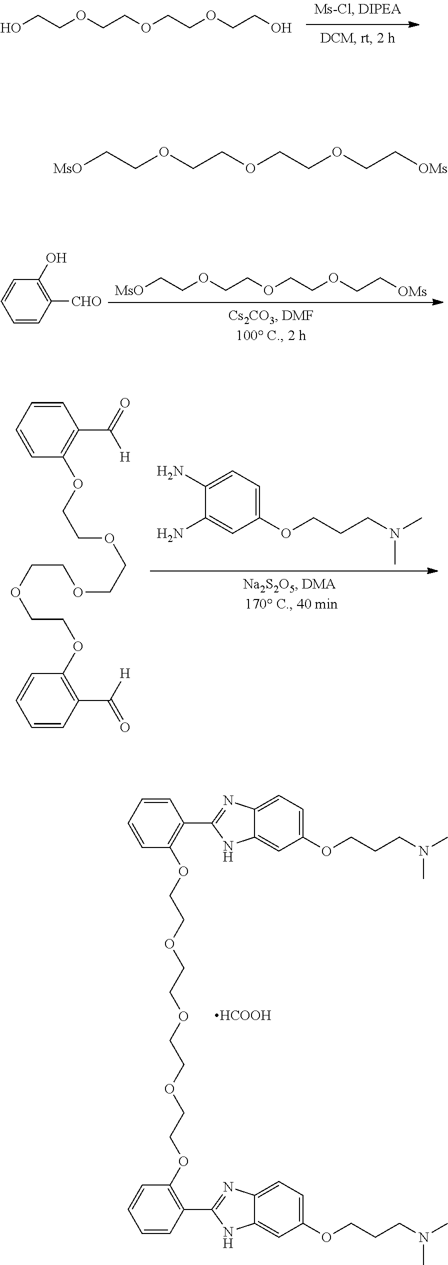

### Synthesis of ((oxybis(ethane-2,1-diyl))bis(oxy))bis(ethane-2,1-diyl) dimethanesulfonate

To a stirred solution of tetraethylene glycol (5.00 g, 25.7 mmol) in CH₂Cl₂ (50.0 mL) at 0° C. under N₂ atmosphere, was added DIPEA (13.5 mL, 77.0 mmol) followed with methane sulfonyl chloride (6.02 mL, 77.0 mmol) and the reaction mixture was stirred at room temperature for 2 h. The contents were poured on to ice cold water, and the aqueous phase was extracted with CH₂Cl₂ (2×50 mL). The combined CH₂Cl₂ extracts were washed with saturated aqueous NaHCO₃ (50 mL), brine (30 mL), dried over anhydrous Na₂SO₄, filtered and concentrated under vacuum to obtain ((oxybis(ethane-2,1-diyl))bis(oxy))bis(ethane-2,1-diyl) dimethanesulfonate.

### Synthesis of 2,2′-((((oxybis(ethane-2, 1-diyl))bis(oxy))bis(ethane-2,1-diyl))bis(oxy))dibenzaldehyde

To a stirred solution of 2-hydroxy benzaldehyde (0.70 g, 5.71 mmol) in anhydrous DMF (20.0 mL), under N₂ atmosphere at room temperature, was added Cs₂CO₃ (2.04 g, 6.28 mmol) followed with ((oxybis(ethane-2,1-diyl))bis(oxy))bis(ethane-2,1-diyl) dimethanesulfonate (1.00 g, 2.85 mmol) and the reaction mixture was stirred at 100° C. for 2 h. The reaction mixture was cooled to room temperature and the contents were poured on to ice cold water, and the aqueous phase was extracted with EtOAc (2×50 mL). The combined EtOAc extracts were washed with brine (50 mL), dried over anhydrous Na₂SO₄, filtered and concentrated under vacuum to obtain the crude product. The product was purified by silica gel chromatography using EtOAc: Hexanes (1:1). The fractions containing the product were combined and concentrated under vacuum to obtain 2,2′-((((oxybis(ethane-2,1-diyl))bis(oxy))bis(ethane-2, 1-diyl))bis(oxy))dibenzaldehyde.

### Synthesis of 3,3′-(((((((oxybis(ethane-2,1-diyl))bis(oxy))bis(ethane-2,1-diyl))bis(oxy))bis(2,1-phenylene))bis(1H-benzo[d]imidazole-2,6-diyl))bis(oxy))bis(N,N-dimethylpropan-1-amine)

In a microwave vial, a mixture of 2,2′-((((oxybis(ethane-2,1-diyl))bis(oxy))bis(ethane-2,1-diyl))bis(oxy))dibenzaldehyde (300 mg, 0.745 mmol), 4-(3-(dimethylamino)propoxy)benzene-1,2-diamine (390 mg, 1.86 mmol) and Na₂S₂O₅ (312 mg, 1.64 mmol) in anhydrous DMA (12.0 mL) was heated at 170° C. for 40 min. The reaction mixture was cooled to r. t and the contents were concentrated under vacuum to obtain the crude product. The product was purified by mass triggered HPLC. The fractions containing only the pure product were combined for concentration to obtain the title compound. MS (ESI): m/z=781 [M+H]+.

### Example 22: Synthesis of 2-hydroxyterephthalaldehyde

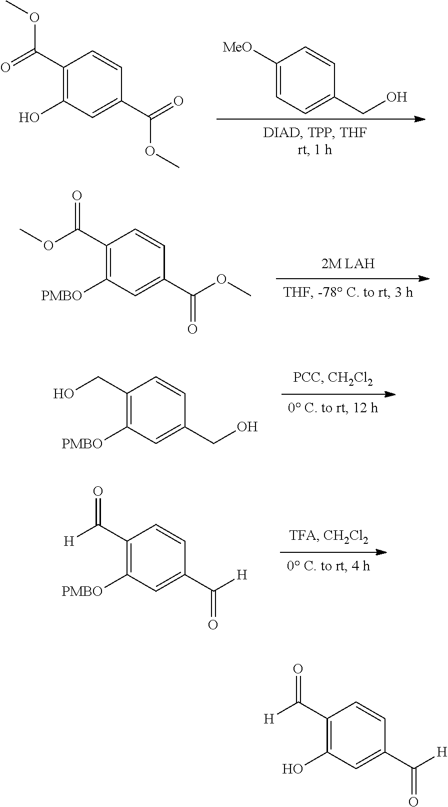

### Synthesis of dimethyl 2-((4-methoxybenzyl)oxy)terephthalate

To a stirred solution of dimethyl 2-hydroxyterephthalate (500 mg, 2.37 mmol) in anhydrous THF (15 mL) at 0° C. under argon, was added (4-methoxyphenyl)methanol (0.35 mL, 2.85 mmol), TPP (936 mg, 3.57 mmol) followed with DIAD (0.70 mL, 3.57 mmol) and the reaction mixture and was stirred at 25° C. for 1 h. The reaction mixture was concentrated under vacuum to obtain the crude product, and the product was purified by silica gel chromatography using EtOAc: Hexanes (3:4). Fractions containing the product were combined for concentration to obtain dimethyl 2-((4-methoxybenzyl)oxy)terephthalate

### Synthesis of (2-((4-methoxybenzyl)oxy)-1,4-phenylene)dimethanol

To a stirred solution of dimethyl 2-((4-methoxybenzyl)oxy)terephthalate (350 mg, 1.06 mmol) in anhydrous THF (4.0 mL), at −78° C. under argon atmosphere, was added LiAIH₄ (2.0 M in THF, 2.65 mL, 5.30 mmol) in a dropwise manner over a period of 10 min. After complete addition, the reaction mixture was gradually allowed to reach r.t, and stirred for 3 h. The reaction mixture was cooled to 0° C. and slowly quenched with satd. aqueous solution of NaSO₄ (5.0 mL), and diluted with water (40 mL). The layers were separated and the aqueous phase was extracted with EtOAc (2×20 mL). The combined EtOAc extracts were washed with water (40 mL), dried over anhydrous Na₂SO₄, filtered and concentrated under vacuum to obtain (2-((4-methoxybenzyl)oxy)-1,4-phenylene)dimethanol.

### Synthesis of 2-((4-methoxybenzyl)oxy)terephthalaldehyde

To a stirred solution of (2-((4-methoxybenzyl)oxy)-1,4-phenylene)dimethanol (170 mg, 0.62 mmol) in anhydrous CH₂Cl₂ (10 mL) at 0° C. was added PCC (801 mg, 3.72 mmol) portion-wise manner over 5 minutes. After complete addition, the reaction mixture was warmed to 25° C. and stirred for 12 h. The reaction mixture was filtered through a celite-pad, and the pad was washed with EtOAc (30 mL). The filtrate was concentrated under vacuum to obtain the crude product. The product was purified by silica gel chromatography using EtOAc: Hexanes (1.5:8.5). Fractions containing the product were combined for concentration to obtain 2-((4-methoxybenzyl)oxy)terephthalaldehyde.

### Synthesis of 2-hydroxyterephthalaldehyde

To a stirred solution of 2-((4-methoxybenzyl)oxy)terephthalaldehyde (160 mg, 0.59 mmol) in anhydrous CH₂Cl₂ (4.0 mL) at r.t 0° C. under N₂ atmosphere, was added TFA (0.20 mL, 2.60 mmol) and the reaction mixture was warmed to 25° C. and stirred for 4 h. The reaction mixture was concentrated under vacuum to obtain the crude product. The product was purified by silica gel chromatography using EtOAc: Hexanes (2:8). Fractions containing the product were combined for concentration to obtain the title compound. MS (ESI): m/z=151 [M+H]⁺.

### Example 23 (Compound A16): Synthesis N,N′-(ethane-1,2-diyl)bis(2-(2,5-bis(6-(3-(dimethylamino)propoxy)-1H-benzo[d]imidazol-2-yl)phenoxy)-N-methylacetamide)

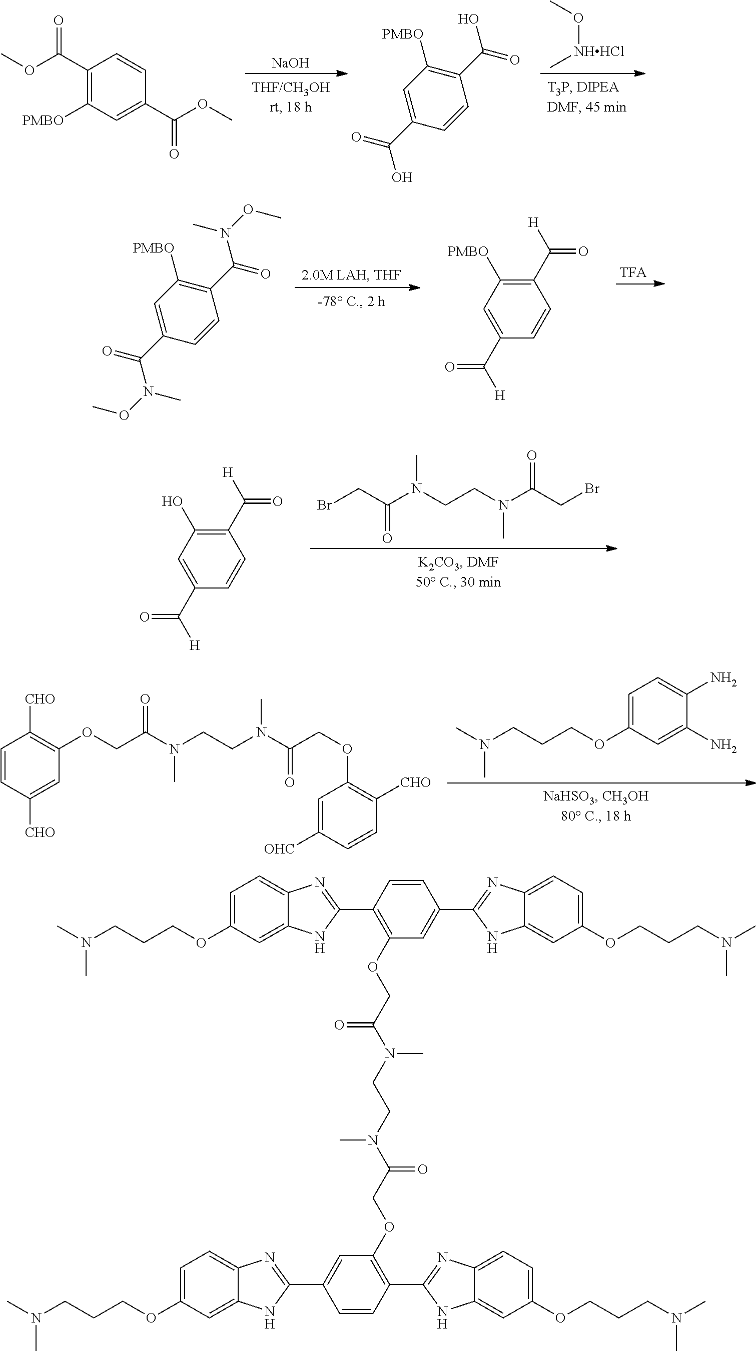

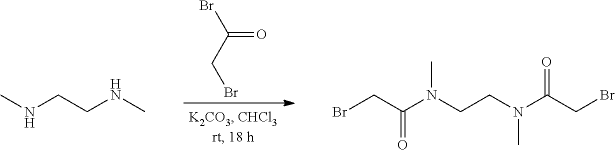

### Synthesis of 2-((4-methoxybenzyl)oxy)terephthalic Acid

To a stirred solution of dimethyl 2-((4-methoxybenzyl)oxy)terephthalate (700 mg, 2.12 mmol) in a mixture of THF (10.0 mL), MeOH (10.0 mL) at r.t was added 3M aqueous NaOH (2.83 mL, 8.48 mmol) and the mixture was stirred at r.t for 2 h. After 2 h, white solids precipitated from the reaction. Added additional amount of H₂O (5.0 mL) and the mixture was stirred at r.t for 4 h; note—the reaction mixture turned homogeneous on adding water. The reaction mixture was concentrated under reduced pressure to remove volatile solvents, and the aqueous phase was acidified to pH 2 using 2N aqueous HCl, whereupon, a white solid precipitated. The solid was filtered under vacuum, washed with water (20 mL), and dried to obtain 2-((4-methoxybenzyl)oxy)terephthalic acid.

### Synthesis of N1,N4-dimethoxy-2-((4-methoxybenzyl)oxy)-N1,N4-dimethylterephthalamide)

To a stirred solution of 2-((4-methoxybenzyl)oxy)terephthalic acid (1.00 g, 3.31 mmol) in anhydrous DMF (20.0 mL) at r.t under N₂ atmosphere, was added DIPEA (2.89 mL, 16.5 mmol), and N,O-dimethylhydroxylamine (0.97 g, 9.92 mmol) followed with 50% T₃P in EtOAc (5.91 mL, 9.92 mmol) and the reaction mixture was stirred at r.t for 45 min. The reaction mixture was poured onto ice cold water (100 mL), and the aqueous phase was extracted with EtOAc (2×100 mL). The combined EtOAc extracts were washed with water (2×150 mL), brine (150 mL), dried over anhydrous Na₂SO₄, filtered and concentrated under vacuum to obtain N1,N4-dimethoxy-2-((4-methoxybenzyl)oxy)-N1,N4-dimethylterephthalamide.

### Synthesis of 2-((4-methoxybenzyl)oxy)terephthalaldehyde)

To a stirred solution of N1,N4-dimethoxy-2-((4-methoxybenzyl)oxy)-N1,N4-dimethylterephthalamide (1.10 g, 2.83 mmol) in dry THF (40.0 mL), at −78° C. under argon atmosphere, was added LiAIH₄ (2.0 M in THF, 5.66 mL, 11.33 mmol) in a dropwise manner over a period of 10 min, and the reaction mixture was stirred at same temperature for 1 h, then slowly warmed to −40° C. and reaction mixture was stirred for additional 1 h. The reaction mixture was slowly quenched with aq. saturated Na₂SO₄ solution at −40° C. and diluted with CH₂Cl₂ (200 mL). The reaction mixture was filtered through celite bed and filterate was concentrated under vacuum to obtain the crude product. The product was purified by silica gel chromatography using EtOAc: Hexanes (2.5:7.5). The fractions containing the product were combined for concentration to obtain 2-((4-methoxybenzyl)oxy)terephthalaldehyde.

### Synthesis of 2-hydroxyterephthalaldehyde

To a stirred solution of 2-((4-methoxybenzyl)oxy)terephthalaldehyde (1.30 g, 4.81 mmol) in anhydrous CH₂Cl₂ (30 mL), at r.t 0° C. under N₂ atmosphere, was added TFA (1.30 mL, 16.8 mmol) and the reaction mixture was warmed to 25° C. and stirred for 4 h. The reaction mixture was concentrated under vacuum to obtain the crude product. The product was purified by silica gel chromatography using EtOAc: Hexanes (2:8). Fractions containing the product were combined for concentration to obtain 2-hydroxyterephthalaldehyde.

### Synthesis of N,N′-(ethane-1,2-diyl)bis(2-bromoacetamide) 8a

To a stirred solution of N1,N2-dimethylethane-1,2-diamine (1.00 g, 11.3 mmol) in anhydrous CHCl₃ (70.0 mL) at r.t, was added a solution of K₂CO₃ (4.71 g, 34.0 mmol) in water (35.0 mL). The mixture was cooled to 5° C., and a solution of 2-bromoacetyl bromide (2.97 mL, 34.0 mmol) in CHCl₃ (70.0 mL) was added in a drop wise manner over period of 10 min. After complete addition, the cooling bath was removed and the reaction mixture was stirred at r.t for 18 h. The organic layer was separated and washed with water (100 mL), brine (100 mL), dried over anhydrous Na₂SO₄, filtered and concentrated under vacuum to obtain N,N′-(ethane-1,2-diyl)bis(2-bromoacetamide).

### Synthesis of N,N′-(ethane-1,2-diyl)bis(2-(2,5-diformylphenoxy)-N-methylacetamide)

To a stirred solution of 2-hydroxyterephthalaldehyde (125 mg, 0.84 mmol) in anhydrous DMF (5.00 mL) at r.t under N₂ atmosphere, was added K₂CO₃ (157 mg, 1.13 mmol) followed with N,N′-(ethane-1,2-diyl)bis(2-bromo-N-methylacetamide) (125 mg, 0.38 mmol) and the reaction mixture was stirred at 50° C. for 30 min. The reaction mixture was concentrated under reduced pressure to obtain the crude product. The product was purified by silica gel chromatography using CH₃OH: CH₂Cl₂ (05:9.5). The fractions containing the product were combined for concentration to obtain tetramethyl N,N′-(ethane-1,2-diyl)bis(2-(2,5-diformylphenoxy)-N-methylacetamide).

### Synthesis of N,N′-(ethane-1,2-diyl)bis(2-(2,5-bis(6-(3-(dimethylamino)propoxy)-1H-benzo[d]imidazol-2-yl)phenoxy)-N-methylacetamide) (Compound A16)

To a stirred solution of N,N′-(ethane-1,2-diyl)bis(2-(2,5-diformylphenoxy)-N-methylacetamide) (75 mg, 0.16 mmol) in anhydrous CH₃OH (20.0 mL) at r.t under N₂ atmosphere, was added NaHSO₃ (83 mg, 0.80 mmol) followed with 4-(3-(dimethylamino)propoxy)benzene-1,2-diamine (168 mg, 0.80 mmol) and the reaction mixture was stirred at 80° C. for 18 h. The reaction mixture was concentrated under reduced pressure to obtain crude product. The product was purified by mass triggered HPLC. The fractions containing only the pure product were combined for concentration to obtain the title compound. MS (ESI): m/z=1226 [M+H]+.

### Example 24 (Compound A12): Synthesis 3,3′-((2,2′-(2-(2-((2-(2,5-bis(6-(3-(dimethylamino)propoxy)-1H-benzo[d]imidazol-2-yl)phenoxy)ethyl)(methyl)amino)ethoxy)-1,4-phenylene)bis(1H-benzo[d]imidazole-6,2-diyl))bis(oxy))bis(N,N-dimethylpropan-1-amine)

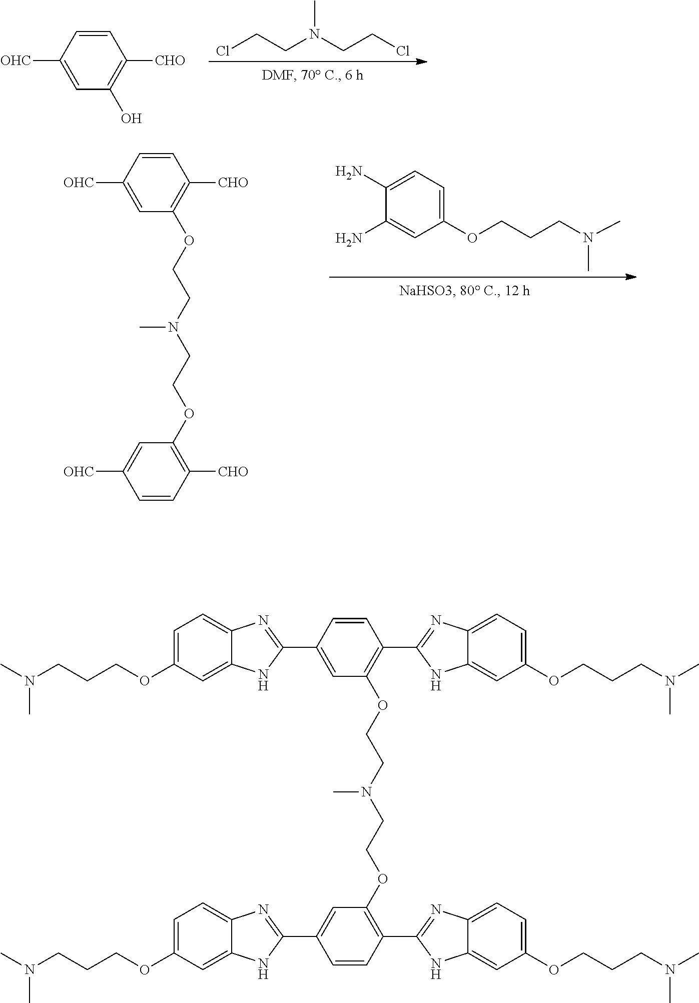

### Synthesis of 2,2′-(((methylazanediyl)bis(ethane-2,1-diyl))bis(oxy))diterephthalaldehyde

To a stirred solution of 2-hydroxy terephthalaldehyde (200 mg, 1.33 mmol) in anhydrous DMF (5.00 mL), at r.t under N₂ atmosphere, was added K₂CO₃ (736 mg, 5.33 mmol) followed with 2-chloro-N-(2-chloroethyl)-N-methylethan-1-amine (128 mg, 0.66 mmol) and the reaction mixture was heated at 70° C. for 6 h. The reaction mixture was cooled to r.t. and the contents were poured onto water and extracted with EtOAc (2×20 mL). The combined organic extracts were washed with brine (30 mL), dried over anhydrous Na₂SO₄, filtered and concentrated under vacuum to obtain the crude product. The product was purified by silica gel chromatography using CH₃OH: CH₂Cl₂ (1:9). The fractions containing the product were combined for concentration to obtain 2,2′-(((methylazanediyl)bis(ethane-2,1-diyl))bis(oxy))diterephthalaldehyde.

### Synthesis 3,3′-((2,2′-(2-(2-((2-(2,5-bis(6-(3-(dimethylamino)propoxy)-1H-benzo[d]imidazol-2-yl)phenoxy)ethyl)(methyl)amino)ethoxy)-1,4-phenylene)bis(1H-benzo[d]imidazole-6,2-diyl))bis(oxy))bis(N,N-dimethylpropan-1-amine) (Compound A12)

To a stirred solution of 2,2′-(((methylazanediyl)bis(ethane-2,1-diyl))bis(oxy))diterephthalaldehyde (100 mg, 0.26 mmol) in anhydrous CH₃OH (5.0 mL) at r.t under N₂ atmosphere, was added NaHSO₃ (136 mg, 1.30 mmol) followed with 4-(3-(dimethylamino)propoxy)benzene-1,2-diamine (273 mg, 1.30 mmol) and the reaction mixture was stirred at 80° C. for 12 h. The reaction mixture was concentrated under reduced pressure to obtain crude product. The product was purified by mass triggered HPLC twice. The fractions containing only the pure product were combined for concentration to obtain the title compound. MS (ESI): m/z=1141 [M+H]+.

### Example 25 (Compound A13): Synthesis 3,3′-((2,2′-(2-(3-((3-(2,5-bis(6-(3-(dimethylamino)propoxy)-1H-benzo[d]imidazol-2-yl)phenoxy)propyl)(methyl)amino)propoxy)-1,4-phenylene)bis(1H-benzo[d]imidazole-6,2-diyl))bis(oxy))bis(N,N-dimethylpropan-1-amine)

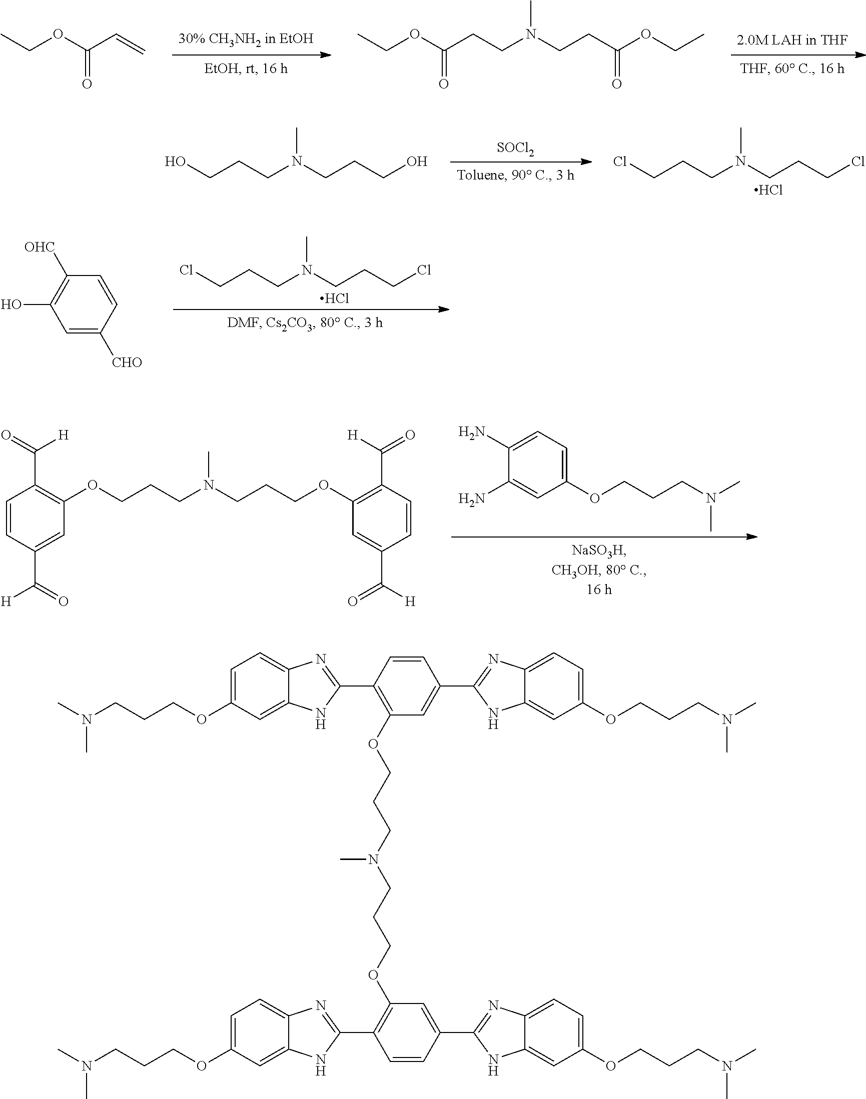

### Synthesis of diethyl 3,3′-(methylazanediyl)dipropanoate

To a stirred solution of ethyl acrylate (5.00 g, 49.9 mmol) in EtOH (20 mL) at 0° C., was added a solution of 30% CH₃NH₂ in EtOH (5.00 mL, 49.9 mmol) and the reaction mixture was stirred at r.t for 16 h. The reaction mixture was concentrated under reduced pressure to obtain the crude product. The product was purified by silica gel chromatography using CH₃OH: CH₂Cl₂ (1:9). The fractions containing the product were combined for concentration to obtain diethyl 3,3′-(methylazanediyl)dipropanoate.

### Synthesis of 3,3′-(methylazanediyl)bis(propan-1-ol)

To a stirred solution of diethyl 3,3′-(methylazanediyl)dipropanoate (1.50 g, 6.49 mmol) in dry THF (15.0 mL), at −78° C. under argon atmosphere, was added 2.0 M LiAIH₄ in THF (32 mL, 64.9 mmol) in a dropwise manner over a period of 10 min, and the reaction mixture was heated at 60° C. for 16 h. The reaction mixture was slowly quenched with aq. saturated Na₂SO₄ solution and diluted with EtOAc (40 mL), and the reaction mixture was filtered through celite bed and filterate was concentrated under vacuum to obtain the crude product. The product was purified by silica gel chromatography using CH₃OH: CH₂Cl₂ (1:9). The fractions containing the product were combined for concentration to obtain 3,3′-(methylazanediyl)bis(propan-1-ol).

### Synthesis of 3-chloro-N-(3-chloropropyl)-N-methylpropan-1-amine Hydrochloride

To a stirred solution of 3,3′-(methylazanediyl)bis(propan-1-ol) (300 mg, 2.03 mmol) in toluene (10 mL), at r.t was added thionyl chloride (0.70 mL, 10.2 mmol) and the reaction mixture was heated at 90° C. for 3 h. The reaction mixture was concentrated under reduced pressure to obtain 3-chloro-N-(3-chloropropyl)-N-methylpropan-1-amine hydrochloride.

### Synthesis of 2,2′-(((methylazanediyl)bis(propane-3-1-diyl))bis(oxy))diterephthalaldehyde

To a stirred solution of 2-hydroxy terephthalaldehyde (140 mg, 0.93 mmol) in anhydrous DMF (10.0 mL), at r.t under N₂ atmosphere, was added Cs₂CO₃ (1.51 g, 4.66 mmol), 3-chloro-N-(3-chloropropyl)-N-methylpropan-1-amine hydrochloride (93 mg, 0.42 mmol) and the reaction mixture was heated at 90° C. for 3 h. The reaction mixture was cooled to r.t. and the contents were poured onto water and extracted with EtOAc (2×20 mL). The combined organic extracts were washed with brine (30 mL), dried over anhydrous Na₂SO₄, filtered and concentrated under vacuum to obtain the crude product. The product was purified by silica gel chromatography using CH₃OH: CH₂Cl₂ (1:9). The fractions containing the product were combined for concentration to obtain 2,2′-(((methylazanediyl)bis(propane-3-1-diyl))bis(oxy))diterephthalaldehyde).

### Synthesis of 3,3′-((2,2′-(2-(3-((3-(2,5-bis(6-(3-(dimethylamino)propoxy)-1H-benzo[d]imidazol-2-yl)phenoxy)propyl)(methyl)amino)propoxy)-1,4-phenylene)bis(1H-benzo[d]imidazole-6,2-diyl))bis(oxy))bis(N,N-dimethylpropan-1-amine)

To a stirred solution of 2,2′-(((methylazanediyl)bis(propane-3-1-diyl))bis(oxy))diterephthalaldehyde (35 mg, 0.08 mmol) in anhydrous CH₃OH (10.0 mL) at r.t under N₂ atmosphere, was added NaHSO₃ (44 mg, 0.43 mmol) followed with 4-(3-(dimethylamino)propoxy)benzene-1,2-diamine (89 mg, 0.42 mmol) and the reaction mixture was stirred at 80° C. for 16 h. The reaction mixture was concentrated under reduced pressure to obtain crude product. The product was purified by mass triggered HPLC. The fractions containing only the pure product were combined for concentration to obtain the title compound. MS (ESI): m/z=1169 [M+H]+.

### Example 26 (Compound A4): Synthesis of 3,3′,3″,3′″-((((propane-1,3-diylbis(oxy))bis(benzene-2,1,4-triyl))tetrakis(1H-benzo[d]imidazole-2,5-diyl))tetrakis(oxy))tetrakis(N,N-dimethylpropan-1-amine)

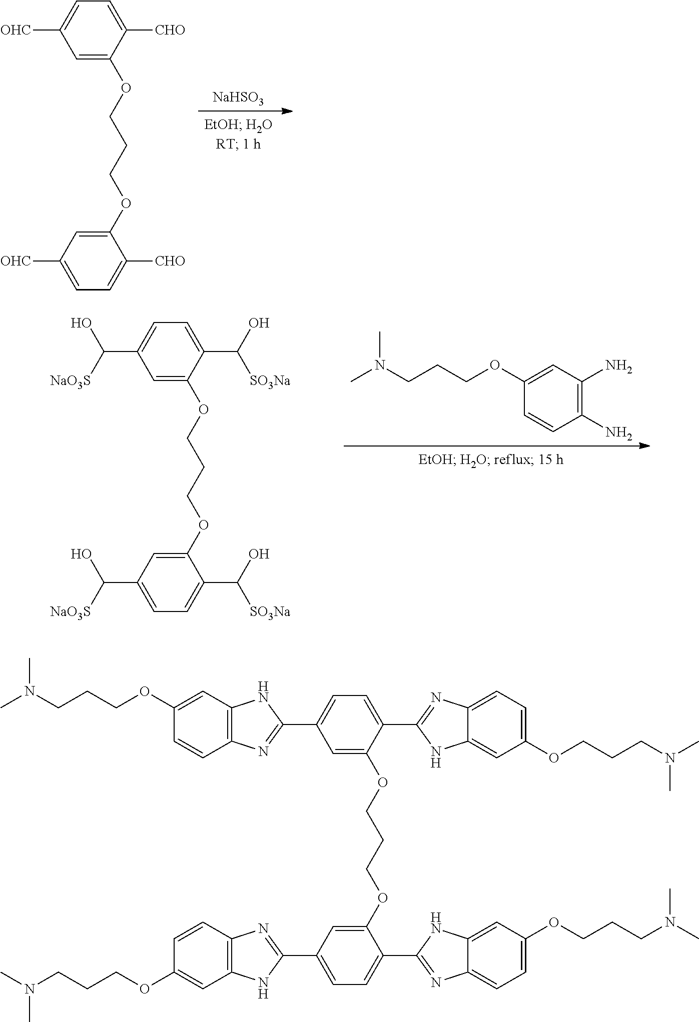

A suspension of 2,2′-(propane-1,3-diylbis(oxy))diterephthalaldehyde (136 mg, 0.4 mmol) in ethanol (8 mL) was taken in a round bottom flask (50 mL) and cooled to 0° C. A solution of sodium bisulfite (183 mg 1.76 mmol) in water (2 mL) was added dropwise in it. The cooling bath was removed, and the reaction mixture was stirred for 1 h at room temperature.

The sulfonate suspension (10 mL) was slowly added to the stirred solution of 4-(3-(dimethylamino) propoxy)benzene-1,2-diamine (368 mg, 1.76 mmol) in water (8 mL) and the reaction mixture was refluxed for 15 h under argon. After cooling to room temperature, the reaction mixture was concentrated under vacuum. The residue was dissolved in water (5 mL) and first purified by reverse phase C18 Biotage column chromatography using a gradient of 0-100% MeOH in water, each containing 0.1% TFA and later by using preparative HPLC using similar gradient over 100 min. method to yield the title compound. LC-MS: (ESI) m/z calculated for C₆₃H₇₈N₁₂O₆[M+2H⁺]/2=549.3, observed 549.5

### Example 27 (Compound A5): Synthesis of 3,3′,3″,3′″-((((dodecane-1,12-diylbis(oxy))bis(benzene-2,1,4-triyl))tetrakis(1H-benzo[d]imidazole-2,5-diyl))tetrakis(oxy))tetrakis(N,N-dimethylpropan-1-amine)

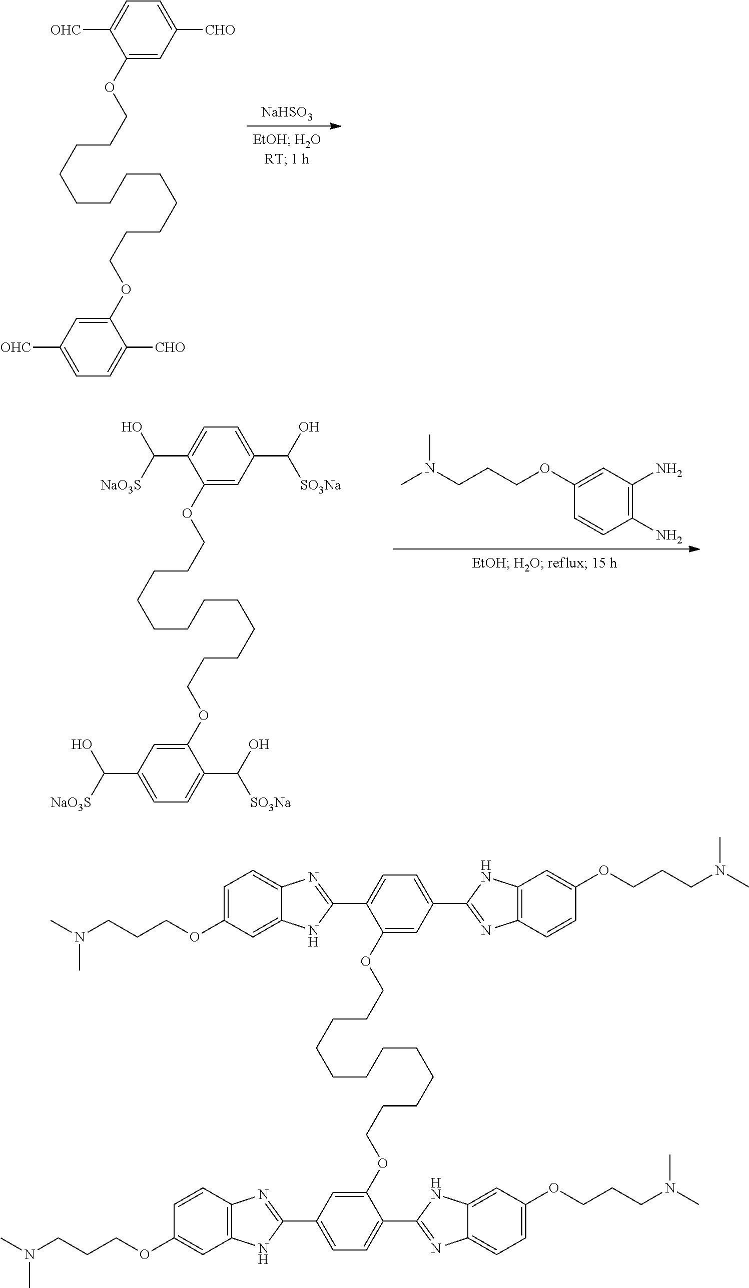

A suspension of 2,2′-(dodecane-1,12-diylbis(oxy))diterephthalaldehyde (280 mg, 0.6 mmol) in ethanol (10 mL) was taken in a round bottom flask (50 mL) and cooled to 0° C. A solution of sodium bisulfite (281 mg 2.70 mmol) in water (2 mL) was added dropwise in it. The cooling bath was removed, and the reaction mixture was stirred for 1 h at room temperature. The sulfonate suspension (12 mL) was slowly added to the stirred solution of 4-(3-(dimethylamino) propoxy)benzene-1,2-diamine (565 mg, 2.7 mmol) in water (10 mL) and the reaction mixture was refluxed for 15 h under argon. After cooling to room temperature, the mixture was filtered, and the filter cake was washed with ethanol (3×20 mL) to give the crude product.

340 mg of crude compound was dissolved in 0.1% TFA water under heating and purified by reverse phase Biotage C18 column chromatography using a gradient of 0-100% MeOH in water, each containing 0.1% TFA to yield the title compound. LC-MS: (ESI) m/z calculated for C₇₂H₉₆N₁₂O₆ [M+2H⁺]/2=612.4, observed 612.6Biological assay data

### Example 28: Small Molecule Inhibition of the r(CUG)₁₂-MBNL1 Complex Measured by Time Resolved Fluorescence Resonance Energy Transfer (TR-FRET)

The in vitro activity of small molecules were assessed by measuring the inhibition of the r(CUG)₁₂-MBNL1 complex using a TR-FRET assay previously reported by Chen et al Analytical and Bioanalytical Chemistry 2012, 402, 1889. Biotinylated r(CUG)₁₂ was folded at 60° C. in 1× Folding Buffer (20 mM HEPES, pH 7.5, 100 mM KCl, and 10 mM NaCl) and slowly cooled to room temperature. The buffer was then adjusted to 1× TR-FRET Buffer (1× Folding Buffer supplemented with 2 mM MgCl₂, 2 mM CaCl₂, 5 mM DTT, 0.1% BSA, 0.05% Tween-20) and MBNL1-His₆ was added. The final concentrations of RNA and MBNL1-His₆ were 80 nM and 60 nM, respectively. Then dilutions of the small molecule were made in 1× TR-FRET buffer. The samples were equilibrated at room temperature for 15 min, after which 1 μL of antibody solution (1:1 mixture of 8.8 ng/μL Anti-His₆-Tb and 800 nM streptavidin XL-665) was added. Samples were incubated at room temperature for 1 h, and then TR-FRET was measured using a Molecular Devices SpectraMax M5 plate reader using an excitation wavelength of 345 nm and a 420 nm cutoff. Controls for maximum TR-FRET (100% complex formation) contained 8 μL of 1× TR-FRET Buffer with RNA and protein and 1 μL water. Controls for minimum TR-FRET (no complex formation) contained 8 μL of 1× Assay Buffer, 1 μL water, and no RNA or protein.

To calculate the percent inhibition of complex formation, the ratio of fluorescence intensity at 545 nm and 665 nm in the presence of compound was compared to the ratio in the absence of small molecule (100% r(CUG)₁₂-MBNL1 complex formation) and in the absence of RNA and protein (no complex formation). The resulting curves were fit to the following equation to determine IC₅₀ values:

$$y = B + \frac{A - B}{1 + {( \frac{IC 50}{x} )}^{hillslope}}$$

where y is ratio of fluorescence intensities at 545 nm and 665 nm (F545/F665), x is the concentration of small molecule, B is F545/F665 value at max FRET effect (solution has RNA and protein but no small molecule added); A is F545/F665 value at min FRET effect (solution has antibodies but no RNA, protein, or small molecule); and the IC50 is the concentration of small molecule where half of the protein is displaced by small molecule.

Small Molecule Inhibition of the r(CAG)₁₂-MBNL1 Complex Measured by Time Resolved Fluorescence Resonance Energy Transfer (TR-FRET). Same procedure as above except the RNA used is 5′ biotinylated r(CAG)₁₂.

Small Molecule Inhibition of the r(CUG)₁₂-MBNL1 Complex Measured by Time Resolved Fluorescence Resonance Energy Transfer (TR-FRET). Same procedure as above with some minor modifications. 5′ biotinylated r(G₄C₂)₈ was folded at 95° C. in 1× Folding Buffer (20 mM HEPES, pH 7.5, and 10 mM NaCl, no KCl) for 2 minutes and slowly cooled to room temperature. Instead of MBNL1-His₆, hnRNP h is used as the RNA-binding protein.

Compounds were tested using this protocol and the results are shown in the below Table B.

TABLE B

| | TR-FRET |
|---|---|
| Ex # | IC₅₀ (μM) |
| 1 | 4.5 |
| 2 | 2.5 |
| 3 | 5.1 |
| 4 | 2.4 |
| 5 | 0.55 |
| 6 | 3.4 |
| 7 | 3.4 |
| 8 | 3.9 |
| 9 | 4.5 |
| 10 | 5.3 |
| 11 | 4.2 |
| 12 | >200 |
| 13 | 188 |
| 14a | 166 |
| 14b | >200 |
| 15 | 48.6 |
| 16 | 2.1 |
| 17 | 2.4 |
| 18 | 0.72 |
| 19 | 1.4 |
| 20 | 2.8 |

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having the structure of Formula I: 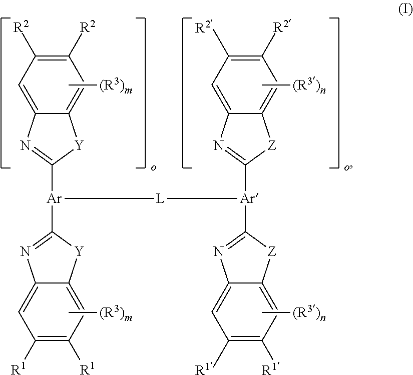
wherein
Ar and Ar′ are independently C₆ aryl or 5-12 membered heteroaryl having 1-4 heteroatoms selected from N, O, and S, wherein Ar and Ar′ are each optionally substituted with 1 to 4 R³;
each R¹, R¹′, R², and R²′ is independently selected from the group consisting of H, C₀₋₃ alkylene-halo, C₀₋₃ alkylene-CN, C₀₋₃ alkylene-NO₂, C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆ alkylene-R⁴;
each R³ and R³′ is independently selected from the group consisting of C₁₋₆alkyl, C₀₋₃ alkylene-halo, C₀₋₃ alkylene-CN, C₀₋₃ alkylene-NH₂, C₀₋₆ alkylene-NH(C═NH)NHR⁸, C₀₋₃ alkylene-OH, and C₀₋₃ alkylene-O—C₁₋₆alkyl;
each R⁴ is independently selected from the group consisting of H, C₁₋₆ alkyl, C₀₋₆alkylene-N(R⁶)₂, C₀₋₆alkylene-N(R⁶)₃⁺, C₀₋₆alkylene-NR⁷—C₀₋₆alkylene-N(R⁶)₂, CH(OR⁶)₂, C₀₋₆alkylene-OR⁶, C₀₋₆alkylene-O—C₀₋₆alkylene-N(R⁶)₂, C₀₋₆alkylene-NR⁶(C═NR⁶)N(R⁶)₂, C₀₋₆alkylene-NR⁶CO₂R⁶, C₀₋₆alkylene-NR⁶COR⁶, CH(R⁷)—C₀₋₆alkylene-NR⁶(C═NR⁶)N(R⁶)₂, and C₀₋₆alkylene-NR⁶C₀₋₆alkylene-NHCO—C₀₋₆alkylene-CO₂R⁶, or two R⁴ together with the nitrogen atom to which they are attached form a 4-7 membered heterocycloalkyl ring optionally further including one additional ring heteroatom selected from O and N;
each R⁶ is independently selected from the group consisting of H, C₁₋₆ alkyl, C₂₋₆ alkenyl, and C₂₋₆ alkynyl, or two R⁶s taken together with the heteroatom(s) to which they are attached form a 4-10 membered heterocycloalkyl ring, optionally having 1 to 3 additional heteroatom ring atom(s) selected from O and N, and said heterocycloalkyl ring is optionally substituted with one to three R⁷;
R⁷ is H, C₁₋₆ alkyl, C₀₋₆ alkyl-OR⁸, or CO₂R⁸;
R⁸ is H or C₁₋₆ alkyl;
each of Y and Z is independently NH or N—C₁₋₃alkyl;
L is —C₁₋₂₀alkylene-, —OC₁₋₂₀alkyleneO—, -(Q-C₁₋₄alkylene)_{y}Q-, —O—C₁₋₄alkylene-Het-C₁₋₄alkyleneO—, or —OC₁₋₄alkyleneO-Het-OC₁₋₄alkyleneO—, wherein the alkylene is optionally substituted with 1-3 groups selected from halo, OR⁸, CN, NO₂, N₃,NR⁸₂, SH, SCH₃, CO₂H, CONH₂, NH(C═NH)NHR⁸, C₆₋₁₀ aryl, 5 to 10-membered heteroaryl having 1-4 ring heteroatoms selected from N, O, and S, C₆₋₁₀ cycloalkyl, and 5 to 12-membered heterocycloalkyl having 1-3 ring heteroatoms selected from N, O, and S; and Het is a C₆aryl or 4-10 membered heterocycloalkyl or heteroaryl ring, having 1 to 4 heteroatom ring atom(s) selected from O, S, and N, and Het is optionally substituted with one to three R⁷;
each Q is independently O, S, NR⁸, CO₂, CONR⁸, NR⁸CO, NR⁸CONR⁸, NR⁸(C═NR⁸)NR⁸, NR⁸SO₂, SO₂, or SON(R⁸)₂;
each m and n is independently 0-2;
each o is independently 0 or 1; and
y is 1-6.

2. The compound or salt of claim 1, wherein Ar and Ar′ are each independently 5-6 membered heteroaryl having 1-4 heteroatoms selected from N, O, and S and are optionally substituted with 1 to 4 R³.

3. The compound or salt of claim 1, wherein Ar and Ar′ are each independently C₆ aryl and are optionally substituted with 1 to 4 R³.

4. The compound or salt of any one of claims 1 to 3, having the structure of Formula Ia, Ib, or Ic:

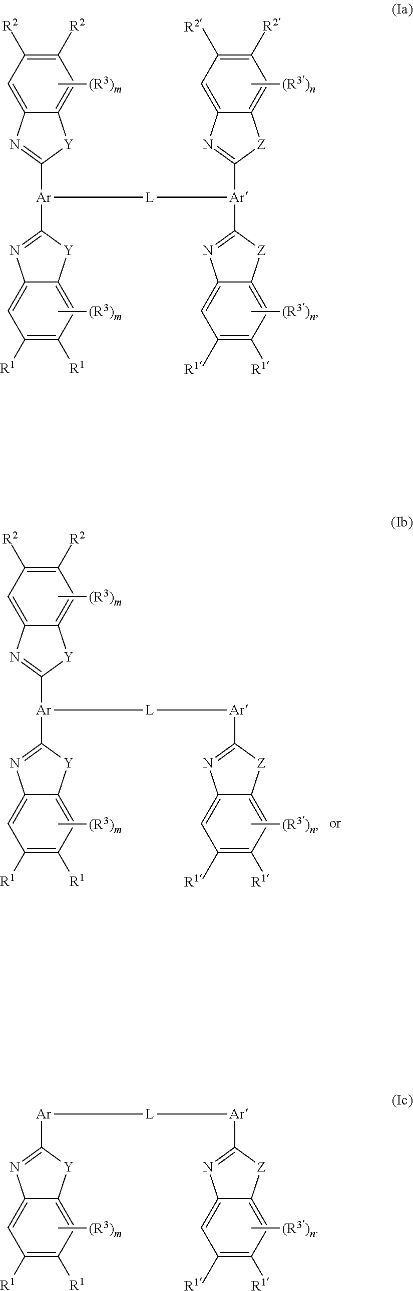

5. The compound or salt of claim 1, having the structure of Formula II:

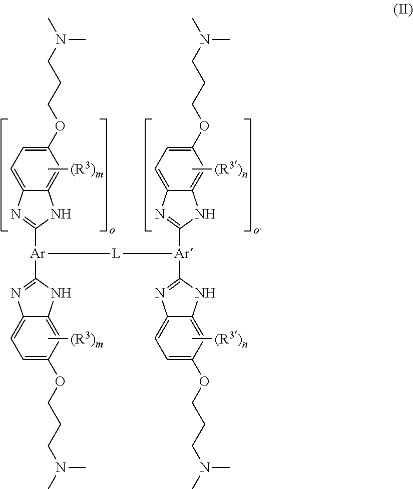

6. The compound or salt of claim 5, having the structure of Formula IIa, IIb, or IIc:

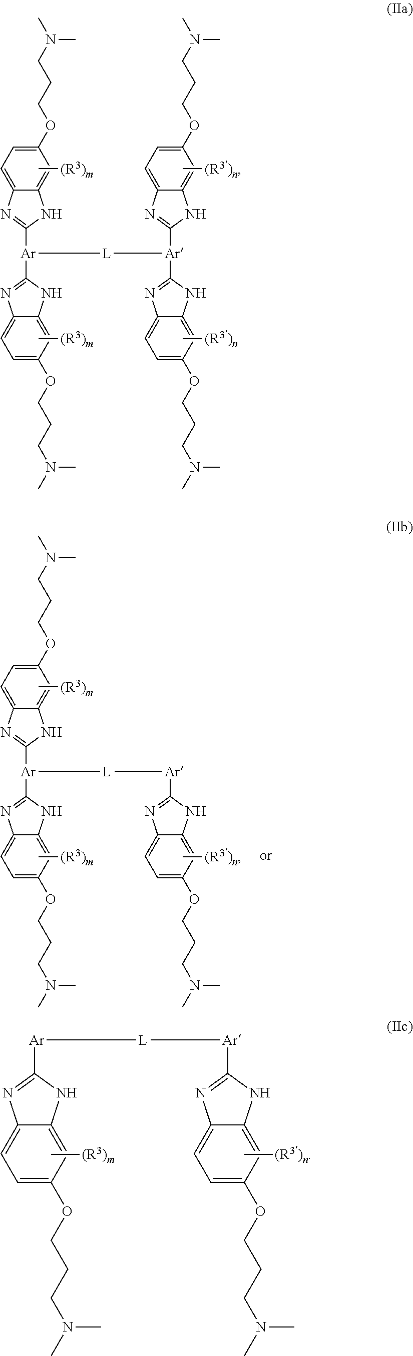

7. The compound or salt of any one of claims 1 to 6, wherein L is 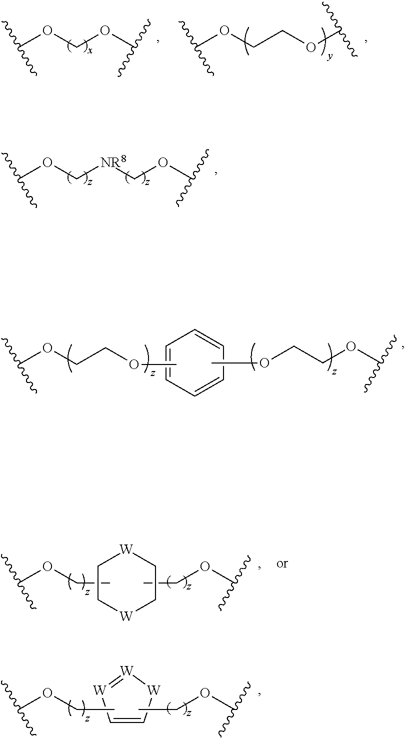
x is 1-14, z is 1-4, and each W is independently O, N, or NR⁸.

8. The compound or salt of claim 7, wherein L is

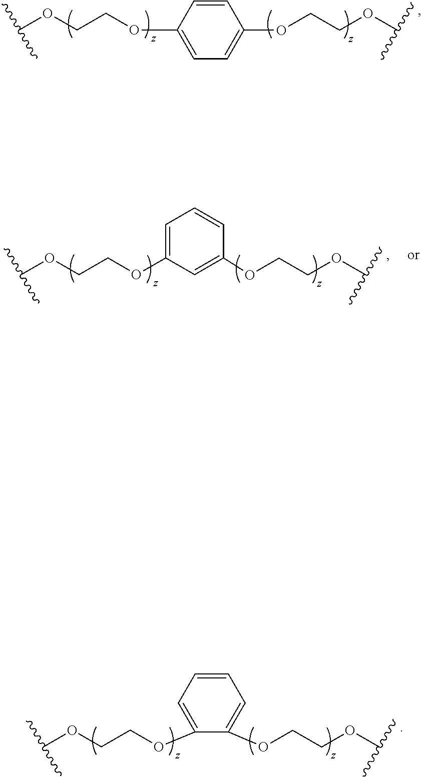

9. The compound or salt of claim 7, wherein L is

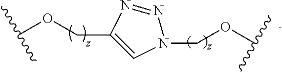

10. The compound or salt of claim 7, wherein L is

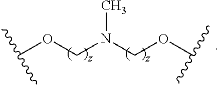

11. The compound or salt of any one of claims 1 to 7, wherein L is —OC₁₋₁₂alkyleneO—.

12. The compound or salt of claim 11, wherein L is C₁₂alkylene.

13. The compound or salt of any one of claims 1 to 7, wherein y is 2-4.

14. The compound or salt of claim 13, wherein y is 2.

15. The compound or salt of claim 13, wherein y is 3.

16. The compound or salt of claim 13, wherein y is 4.

17. The compound or salt of any one of claims 1 to 10, wherein L is —OC₁₋₃alkylene —NR⁸—C₁₋₃alkyleneO—, —O—C₁₋₃alkylene-Het-C₁₋₃alkyleneO—, or —OC₁₋₃alkyleneO-Het-OC₁₋₃alkyleneO-.

18. The compound or salt of claim 17, wherein L is L is —OC₂alkylene —NR⁸—C₂alkyleneO—, —O—C₂alkylene-Het-C₂alkyleneO—, or —OC₂alkyleneO-Het-OC₂alkyleneO-.

19. The compound or salt of any one of claims 1 to 18, wherein each m and n are 1.

20. The compound or salt of any one of claims 1 to 18, wherein each m and n are 2.

21. The compound or salt of any one of claims 1 to 18, wherein at least one m or n is 0.

22. The compound or salt of any one of claims 1 to 4 and 7 to 21, wherein each Y and Z is independently NH or NCH₃.

23. The compound or salt of any one of claims 1 to 4 and 7 to 22, wherein at least one Y or Z is NH.

24. The compound or salt of any one of claims 1 to 4 and 7 to 23, wherein one R¹ or R¹′ is selected from the group consisting of C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆ alkylene-R⁴, and the other R¹ or R¹′ is selected from the group consisting of H, C₀₋₃ alkylene-halo, C₀₋₃ alkylene-CN, C₀₋₃ alkylene-NO₂, C₀₋₆ alkylene-OR⁴, and C₀₋₆ alkylene-SR⁴.

25. The compound or salt of any one of claims 1 to 4 and 7 to 24, wherein one R² or R²′ is selected from the group consisting of C₀₋₆ alkylene-CO₂R⁴, C₀₋₆ alkylene-COR⁴, C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NH(C═NH)NHR⁴, C₀₋₆ alkylene-NHCONHR⁴, C₀₋₆ alkylene-(C═NR⁴)NHR⁴, C₀₋₆ alkylene-NHCOR⁴, and C₀₋₆alkylene-R⁴, and the other R² or R²′ is selected from the group consisting of H, C₀₋₃ alkylene-halo, C₀₋₃alkylene-CN, C₀₋₃ alkylene-NO₂, C₀₋₆ alkyl-OR⁴, and C₀₋₆ alkyl-SR⁴.

26. The compound or salt of any one of claims 1 to 4 and 7 to 25, wherein each R¹, R¹′, R², and R²′ is independently selected from the group consisting of H, C₀₋₃ alkylene-NH₂, C₀₋₆ alkylene-CON(R⁴)₂, C₀₋₆ alkylene-N(R⁴)₂, and C₀₋₆ alkylene-NH(═NH)NHR⁴.

27. The compound or salt of any one of claims 1 to 4 and 7 to 26, wherein R⁴ is selected from the group consisting of C₀₋₆alkylene-N(R⁶)₂, C₀₋₆alkylene-N(R⁶)₃⁺, C₀₋₆alkylene-NR⁷—C₀₋₆alkylene-N(R⁶)₂, and C₀₋₆alkylene-O—C₀₋₆alkylene-N(R⁶)₂.

28. The compound or salt of any one of claims 1 to 4 and 7 to 26, wherein each R⁴ is independently selected from the group consisting of H, C₁₋₆ alkyl, C₂₋₆alkylene-N(R⁶)₂, C₂₋₆alkylene-N(R⁶)₃⁺, C₂₋₆alkylene-NR⁷—C₂₋₆alkylene-N(R⁶)₂, CH(OR⁶)₂, C₂₋₆alkylene-OR⁶, C₂₋₆alkylene-O—C₂₋₆alkylene-N(R⁶)₂, C₂₋₆alkylene-NR⁶(C═NH)N(R⁶)₂, C₂₋₆alkylene-NR⁶CO₂R⁶, C₂₋₆alkylene-NR⁶COR⁶, CH(R⁷)—C₂₋₆alkylene-NR⁶(C═NH)N(R⁶)₂, and C₂₋₆alkylene-NR⁶C₂₋₆alkylene-NHCO—C₂₋₆alkylene-CO₂R⁶.

29. The compound or salt of any one of claims 1 to 4 and 7 to 23, wherein at least one R¹, R¹′ R², or R²′ comprises C₀₋₆ alkylene-CON(R⁴)₂, C₀-6 alkylene-OR⁴, C₀₋₆ alkylene-N(R⁴)₂, C₀₋₆ alkylene-SR⁴, C₀₋₆ alkylene-NHCOR⁴, C₀₋₆ alkylene-R⁴, and R⁴ comprises C₀₋₆alkylene-N(R⁶)₂, C₀₋₆alkylene-N(R⁶)₃⁺, C₀₋₆alkylene-NR⁷—C₀₋₆alkylene-N(R⁶)₂, or C₀₋₆alkylene-O—C₀₋₆alkylene-N(R⁶)₂.

30. The compound or salt of claim 29, wherein one R¹ or R¹′ and one R² or R²′ each independently comprise C₀₋₆ alkylene-CON(R⁴)₂, C₀₋₆ alkylene-OR⁴, C₀₋₆ alkylene-N(R⁴)₂, C₀₋₆ alkylene-SR⁴, C₀₋₆ alkylene-NHCOR⁴, C₀₋₆ alkylene-R⁴, and R⁴ comprises C₀₋₆alkylene-N(R⁶)₂, C₀₋₆alkylene-N(R⁶)₃⁺, C₀₋₆alkylene-NR⁷—C₀₋₆alkylene-N(R⁶)₂, or C₀₋₆alkylene-O—C₀₋₆alkylene-N(R⁶)₂.

31. The compound or salt of claim 29 or 30, wherein R⁶ comprises H or C₁₋₆alkyl, or two R⁶s taken together with the heteroatom(s) to which they are attached form a 4-10 membered heterocycloalkyl ring, optionally having 1 to 3 (e.g., 1-2, 1, 2, or 3) additional heteroatom ring atom(s) selected from O and N, and said heterocycloalkyl is optionally substituted with 1 or 2 R⁷.

32. A compound, as recited in Table A, or pharmaceutically acceptable salt thereof.

33. A pharmaceutical composition comprising the compound or salt of any one of claims 1 to 32 and a pharmaceutically acceptable carrier or excipient.

34. A method of treating or preventing a nucleotide repeat disorder comprising administering to a subject in need thereof a therapeutically effective amount of the compound or salt of any one of claims 1 to 33 or the pharmaceutical composition of claim 33.

35. The method of claim 34, wherein the nucleotide repeat disorder is Myotonic Dystrophy Type 1, Myotonic Dystrophy Type 2, or Fuchs dystrophy.

36. The method of claim 34, wherein the nucleotide repeat disorder is Huntington Disease, Amyotrophic Lateral Sclerosis, or Frontotemporal Dementia.

37. The method of any one of claims 34 to 36, wherein the subject is human.
